(19) United States
(12) Patent Application Publication
(10) Pub. No.: US 2014/0073631 A1
(43) Pub. Date: Mar. 13, 2014

(54) ANTIVIRAL AND ANTIMICROBIAL COMPOUNDS

(71) Applicant: Vymed Corporation, Germantown, MD (US)

(72) Inventor: B. Vithal SHETTY, Germantown, MD (US)

(73) Assignee: Vymed Corporation, Germantown, MD (US)

(21) Appl. No.: 14/022,433

(22) Filed: Sep. 10, 2013

Related U.S. Application Data
(60) Provisional application No. 61/700,029, filed on Sep. 12, 2012.

(51) Int. Cl. C07D 215/56; C07D 498/06; C07D 513/04; C07D 401/14; C07D 471/04; C07D 487/08; C07D 487/04

(52) U.S. Cl. CPC C07D 215/56; C07D 487/08; C07D 498/06; C07D 487/04; C07D 401/14; C07D 471/04; C07D 513/04
USPC 514/215; 514/224.5; 514/230.2; 514/235.2; 514/252.11; 514/253.04; 514/253.08; 514/300; 540/474; 540/580; 544/32; 544/101; 544/121; 544/279; 544/357; 544/362; 544/363; 546/113; 546/123

(57) ABSTRACT

Disclosed are guanidine and biguanidine derivatives which have anti-viral and antibacterial activity. Also disclosed are pharmaceutical compositions containing such compounds as an active ingredient, and anti-viral and anti-bacterial methods utilizing such compounds. Methods of treating infections using the guanidine and biguanidine derivatives are also disclosed.

This application claims the benefit of Patent Application No. 61/700,029 filed on Sep. 12, 2012, the disclosure of which is incorporated herein in its entirety by reference.

### BACKGROUND OF THE INVENTION

1. Field of the Invention

The present invention relates to compounds that are active as antiviral and anti-microbial agents against various diseases and methods of treating patients suffering from such diseases.

2. Description of the Related Art

Quinolones and azaquinolones are well known antibacterial agents which have been commercially available for more than 20 years under various names like Nalidixic acid, ciprofloxacin, ofloxacin, norfloxacin, lomefloxacin, enoxacin, sparfloxacin, pefloxacin and others. The primary use of these agents is to treat bacterial infection. The first quinolone compound was synthesized in 1976 and its mechanism of action was discovered in 1976, Gellert et al. (Proc. Natl. Acad. Sci., U.S.A., 73, 3872 (1976)). The mode of action of the quinolones is through the inhibition of the bacterial DNA gyrase enzyme. This essential enzyme is a bacterial type II topoisomerase which controls DNA topology and assists with DNA replication, repair, decatenation and transcription. The bactericidal nature of quinolones and azaquinolones is due to their potent inhibition of bacterial DNA gyrase, an enzyme which regulates the supercoiling, uncoiling and spatial geometry of bacterial DNA functions that are necessary for controlling DNA replication and repair, transcription and recombination. Quinolones appear to induce a cellular repair mechanism in these same organisms, leading to unbalanced growth and alteration of cellular structure, which results in the death of the bacterial cell.

DNA gyrase is a type 2 topoisomerase, which has 2A subunits and 2B subunits. These subunits are the target proteins for the quinolones. Mammalian topoisomerase II is highly resistant to inhibition by the quinolones and azaquinolones. Furthermore, the eukaryotic enzyme differs from DNA gyrase by structurally and functionally removing, as opposed to inducing, supertwists into DNA. The bacterial gyrase enzyme is approximately 100 times more sensitive to inhibition than the eukaryotic equivalent.

Quinolones have a paradoxical effect of decreased killing at higher drug concentration. This is because of the fact that at high doses, quinolones inhibit RNA synthesis and protein synthesis. This paradoxical effect of quinolones and azaquinolones are important in designing target compounds against the AIDS virus.

Tetraaza macrocyclic ligands ranging from 12 members to 16 members are known in the literature. For example, the preparation of macrocycles (cyclam) are reported by L. Y. Martin, et al., in J. Am. Chem. Soc., 96, 4046 (1974) and J. Am. Chem. Soc. 99, 2968 (1997). Cyclam is also commercially available from Aldrich. The preparation of bis(macrocycles) is reported by Barefield, E. K., et al., in J. Chem. Soc. Chem. Commun., 302-304 (1981) and Cimpolini, M. et al., in Inorg. Chem., 26, 3527-3533 (1987). These broad families of synthetic macrocyclic and bis(macrocyclic) ligands have been studied not only for their complexation properties with metals but also for their antiviral activities. In the present work, these ligands are used as a template for the guanidine and biguanidine derivatives for their dual mechanism of action as anti-HIV and anti-bacterial activities.

The core structure of the AIDS virus is protected by a lipid layer and highly glycosylated protein (gp 160) which endoproteolytically splits into gp 41 and gp 120. The latter protein binds to CD₄ of the T lymphocyte and converts the virus RNA into DNA with the help of the enzyme, reverse transcriptase. The highly balanced hydrophilic-hydrophobic site-specific compounds of the present invention possibly penetrate the viral envelope to reach to the core structure of the AIDS virus and thus act as virucidal-bactericidal agents.

The acquired immunodeficiency syndrome (AIDS) is caused by human immunodeficiency virus (HIV) which is also known by several names, namely HIV-1, LAV (lymphadenopathy-associated virus), HTLV-3 (human T-lymphotropic virus type 3) and ARV (AIDS related virus). The genomes of HIV-1 and HIV-2 are only about 50% homologous at the nucleotide level. Yet the two viruses contain the same complement of genes and appear to attack and kill the same human cells by much the same mechanism. In the US, all the AIDS cases are associated with HIV-1 infections. Since the two viruses, HIV-1 and HIV-2 share similar biological properties, they have similar mechanism of action. Both HIV-1 and HIV-2 are retroviruses in which the genetic material is RNA rather than DNA. HIV-1 and HIV-2 viruses may not necessarily cause the death of a patient, but they do, in many cases, cause the patient's immune system to be severely depressed. This results in various other diseases (secondary infection or tumor formation) such as various bacterial infections (e.g., pneumocystis carinii pneumonia) herpes, cytomegalovirus, Kaposi Sarcoma and Epstein-Barr virus related lymphomas among others. These secondary infections are generally referred to as opportunistic infections. They are separately treated using other medications. There is thus a great need for drugs which are capable of both treating the underlying HIV infection, as well as secondary opportunistic infections.

Bacillus anthracis is a bacterium that causes anthrax. It is different from other bacteria as it is a gram positive spore and may become deadly under certain conditions. It is an aerobic (oxygen requiring) bacterium that lives in soil. The hardiness and toxicity of Bacillus anthracis make it a possible bioterrorism agent. Infection can also occur in humans when they are exposed to infected animals. Its ability to produce toxin make it potent killer. The toxin is made of three proteins: protective antigen, edema factor and lethal factor.

Yersinia pestis is the gram negative bacterium that causes plague. It can grow with or without oxygen. It is found most commonly in rats but other animals like prairie dogs and fleas also carry it. The bacteria can survive for months in cool, moist conditions. The bacterium causes an infectious disease of animals and humans.

Burkholderia pseudomallei is a gram negative non-spore fanning organism and is a potential agent for biological warfare and biological terrorism. Melioidosis also called Whitmore's disease is a predominantly tropical infectious disease caused by the bacterium that may infect humans or animals. The bacteria are found in contaminated water and soil. It causes acute or localized infection, acute pulmonary, and bloodstream infections.

Francisella tularensis is considered to be a dangerous potential bioterrorist threat. Tularemia had been used in warfare as biological weapon. It is a hardy organism capable of surviving in low temperature in water, moist soil and other conditions. It is a gram negative bacterium that is highly virulent both in humans and animals. Tularemia is typically found in animals like rodents and rabbits. Skin ulcers, inflamed glands, fever and other symptoms develop in infected persons. Tularemia poses a serious concern as biological weapon mainly because it is one of the most infectious pathogenic bacteria known. Inhalation of as few as 10 bacteria can cause disease and it has substantial capacity to cause serious illness and death.

Burkholderia pseudomallei causes the infectious disease glanders and is a potential agent for biological warfare and of biological terrorism. It was used by Germany during World War I and again in World War II by the Japanese forces. It was also used against mujahideen in Afghanistan in the 1980's. The bacteria are transmitted to human through contact with tissues or body fluids of infected animals. It causes pulmonary, blood, and other types of infections.

An estimated one third of the world's population is infected with Mycobacterium tuberculosis (TB) and nearly 9 million persons develop tuberculosis each year. It is spread most commonly by airborne transmission and often lung is the target. TB is the leading cause of mortality among persons infected with HIV-1 (AIDS).

There is thus a great need for drugs which are capable of safely and effectively treating, preventing, and inhibiting various viral and bacterial infections.

### SUMMARY OF THE INVENTION

In one aspect, the present invention relates to a compound having one of the following structures:

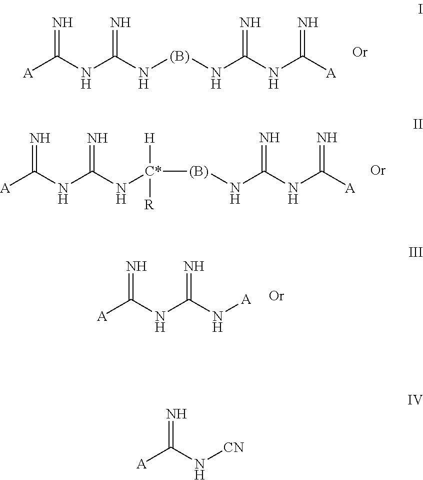

wherein:
a) each A is the same or different, and is selected from the group consisting of i) hydrogen, ii) a nitrile, iii) an amino, iv) an antibacterial agent, v) an antibiotic, vi) a quinolone, vii) an azaquinolone and viii) one of the following groups:

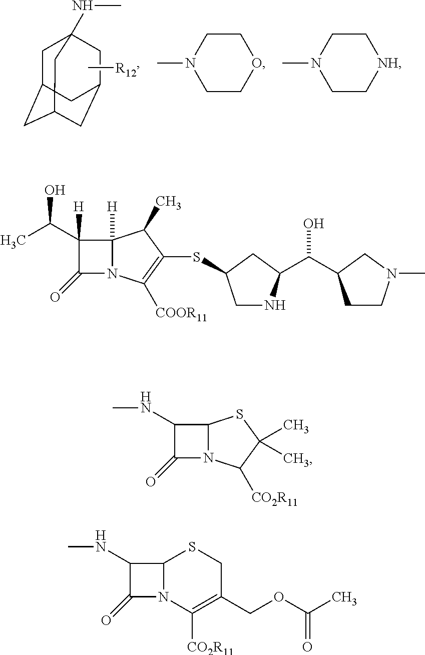

where R₁₂ is hydrogen or C₁-C₆ straight or branched alkyl, and R₁₁ is hydrogen, lower alkyl, an aromatic group or a heterocyclic group, with the proviso that with respect to structures I-III, both A's cannot be the adamantane structure above at the same time;
b) B is a straight chain or branched C₁-C₃₀ alkyl group, which may be interrupted by oxygen, sulfur, optionally substituted aromatic nuclei, sulphoxide, optionally substituted cyclohexane, nitrogen optionally substituted with —NH—C(NH)—NH—C(NH)-A where A is defined above, tris (2-aminoethyl)amine, a heterocycle of the following structure:

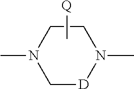

where D is 1-3 carbon atoms and Q is hydrogen, halogen or lower alkyl; or

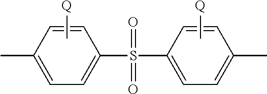

where Q is hydrogen, halogen or lower alkyl; or

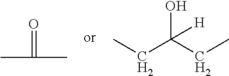

or a hydrophilic moiety; and
   c) R is hydrogen or C₁-C₆ straight or branched alkyl;

and pharmaceutically acceptable salts thereof.

In another aspect, the present invention relates to a compound having the following structure:

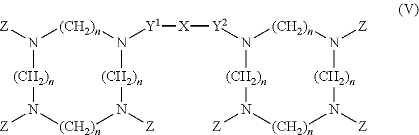

where each n is independently from 1-5;

Y¹ and Y² are the same or different, and are optionally substituted alkyl; optionally substituted aryl; an optionally substituted heterocycle; or a single bond;

X is optionally substituted alkyl; optionally substituted aryl; or an optionally substituted heterocycle; and

Z is independently —C(NH)—NH—C(NH)-A,

where each A is the same or different, and is selected from the group consisting of i) hydrogen, ii) a nitrile, iii) an amino, iv) an antibacterial agent, v) an antibiotic, vi) a quinolone, vii) an azaquinolone and viii) one of the following groups:

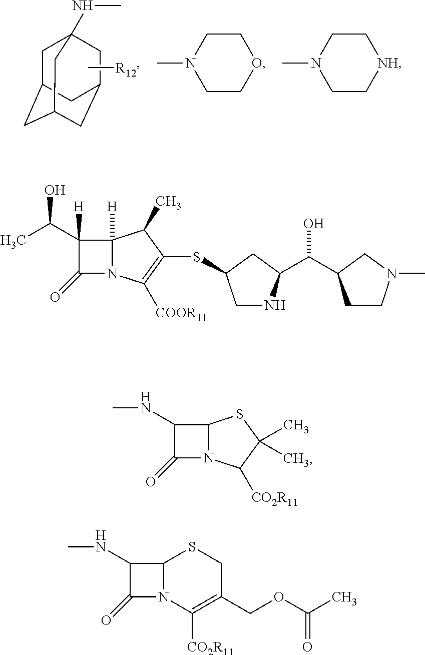

where R₁₂ is hydrogen or C₁-C₆ straight or branched alkyl, and R₁₁ is hydrogen, lower alkyl, an aromatic group or a heterocyclic group;

and pharmaceutically acceptable salts thereof.

in another aspect, the present invention relates to a compound having the following structure:

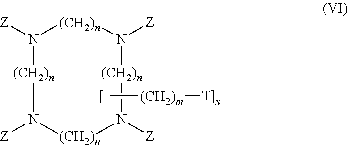

where each n is independently from 1-5;

each m is independently from 0-12;

each Z is independently —C(NH)NH—C(NH)-A, where each A is the same or different, and is selected from the group consisting of i) hydrogen, ii) a nitrile, iii) an amino, iv) an antibacterial agent, v) an antibiotic, vi) a quinolone, vii) an azaquinolone and viii) one of the following groups:

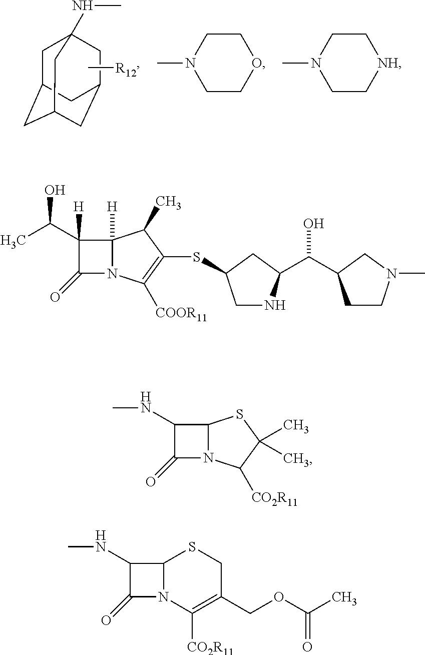

where R₁₂ is hydrogen or C₁-C₆ straight or branched alkyl, and R₁₁ is hydrogen, lower alkyl, an aromatic group or a heterocyclic group,

T is hydrogen, lower alkyl, optionally substituted aryl or optionally substituted heterocycle; and X is from 0-8.

In another aspect, the present invention relates to a compound having the following structure:

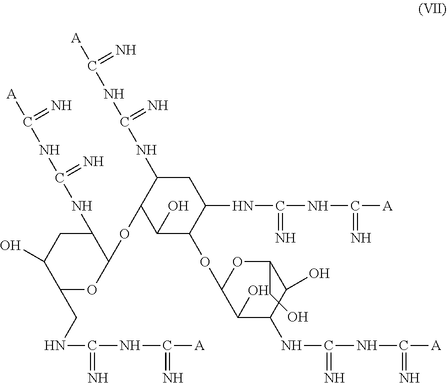

where each A is the same or different, and is selected from the group consisting of i) hydrogen, ii) a nitrile, iii) an amino, iv) an antibacterial agent, v) an antibiotic, vi) a quinolone, vii) an azaquinolone and viii) one of the following groups:

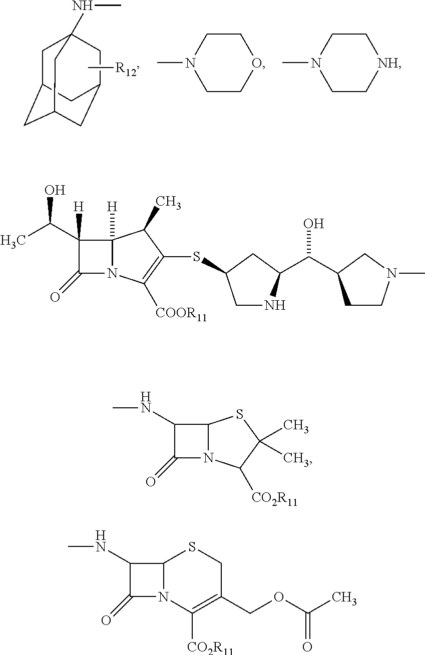

where R₁₂ is hydrogen or C₁-C₆ straight or branched alkyl, and R₁₁ is hydrogen, lower alkyl, an aromatic group or a heterocyclic group with the proviso that at least one A is not hydrogen.

In another aspect, the present invention relates to a compound having the following structure:

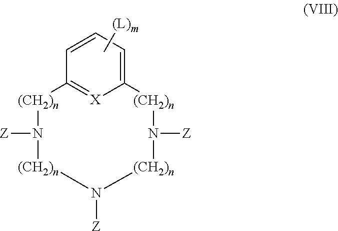

wherein each n is independently from 1-5;

m is from 0-3;

each L is independently hydrogen, lower alkyl, optionally substituted aryl, or nitro;

X is CH or N;

each Z is independently —C(NH)NH—C(NH)-A where

each A is the same or different, and is selected from the group consisting of i) hydrogen, ii) a nitrile, iii) an amino, iv) an antibacterial agent, v) an antibiotic, vi) a quinolone, vii) an azaquinolone and viii) one of the following groups:

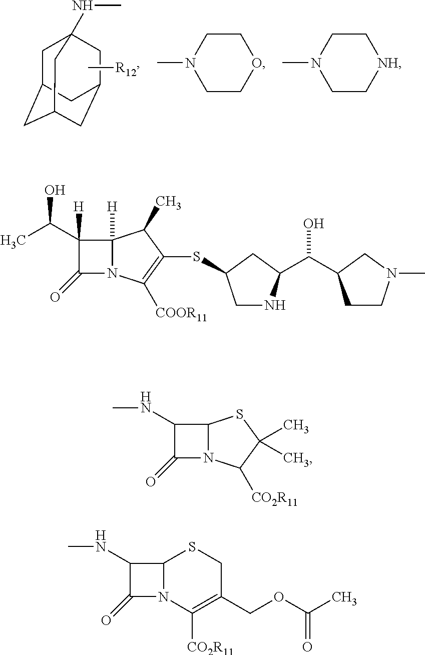

where R₁₂ is hydrogen or C₁-C₆ straight or branched alkyl, and R₁₁ is hydrogen, lower alkyl, an aromatic group or a heterocyclic group.

In another aspect, the present invention relates to a compound having the following structure:

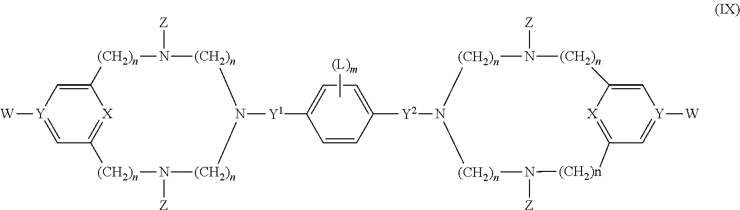

where m is from 0-4;

each L is independently hydrogen, halogen, alkyl, aryl or nitro;

each W is independently hydrogen, halogen, alkyl, alkoxy, or aryl;

X and Y are each independently CH or N;

Y¹ and Y² are each independently optionally substituted alkyl or a single bond; and

each Z is independently —C(NH)—NH—C(NH)-A,

where each A is the same or different, and is selected from the group consisting of i) hydrogen, ii) a nitrile, iii) an amino, iv) an antibacterial agent, v) an antibiotic, vi) a quinolone, vii) an azaquinolone, and viii) one of the following groups:

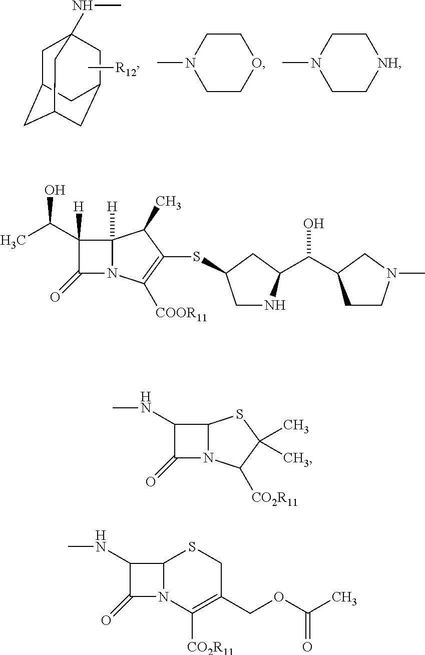

where R₁₂ is hydrogen or C₁-C₆ straight or branched alkyl, and R₁₁ is hydrogen, lowered alkyl, an aromatic group or a heterocyclic group;

and pharmaceutically acceptable salts thereof.

In another aspect, the present invention relates to an antiviral composition which comprises:

a) an effective amount of a compound described above; and

b) a pharmaceutically acceptable carrier.

In another aspect, the present invention relates to an antibacterial composition which comprises:

a) an effective amount of a compound described above; and

b) a pharmaceutically acceptable carrier.

In another aspect, the present invention relates to an antiviral and antibacterial composition which comprises:

a) an effective amount of a compound described above; and

b) a pharmaceutically acceptable carrier.

In another aspect, the present invention relates to a method for preventing or treating a bacterial infection in a mammalian host, said method comprising administering to a mammal in need thereof an effective amount of a compound described above.

In another aspect, the present invention relates to a method for preventing or treating a viral infection in a mammalian host, said method comprising administering to a mammal in need thereof an effective amount of a compound described above.

In another aspect, the present invention relates to a method for preventing or treating a viral infection and an opportunistic bacterial infection in a mammalian host, said method comprising administering to a mammal in need thereof an effective amount of a compound described above.

In another aspect, the present invention relates to compounds useful in the synthesis of the compounds of structure (v) above, and which compounds have the following structure:

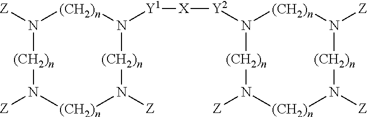

where each n is independently from 1-5;
Y¹ and Y² are the same or different, and are optionally substituted alkyl; optionally substituted aryl; an optionally substituted heterocycle; or a single bond;

X is optionally substituted alkyl; optionally substituted aryl; or an optionally substituted heterocycle; and

each Z is independently —C(NH)—NH—CN or —(CH₂)ₘ—NH—C(NH)—NH—CN where m is from 1 to 6.

In another aspect, the present invention relates to compounds useful in the synthesis of the compounds of structure (VI) above, which compounds have the following structure:

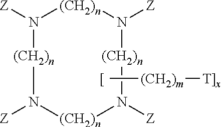

where each n is independently from 1-5;

each m is independently from 0-12;

each Z is independently —C(NH)—NH—CN or —(CH₂)_{q}—NH—C(NH)—NH—CN where q is from 1 to 6, and

T is hydrogen, lower alkyl optionally substituted aryl or an optionally substituted heterocycle; and X is from 0-8.

### BRIEF DESCRIPTION OF THE DRAWINGS

FIG. 1 is a graph depicting the antiviral activity of a prior art compound.

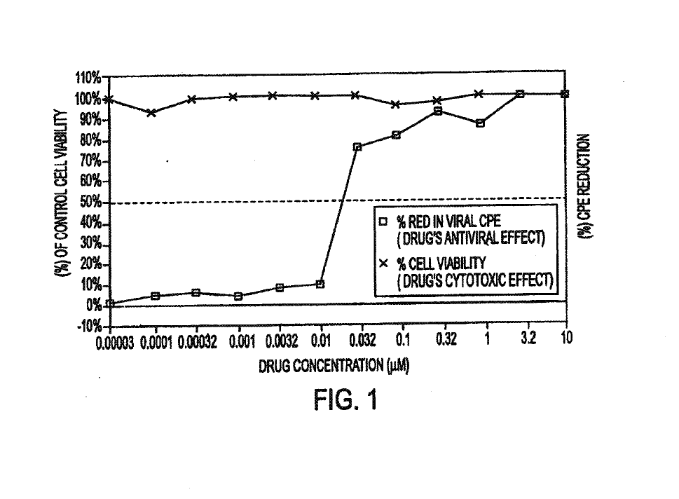

FIG. 2 is a graph depicting the antiviral activity of a compound of the present invention.

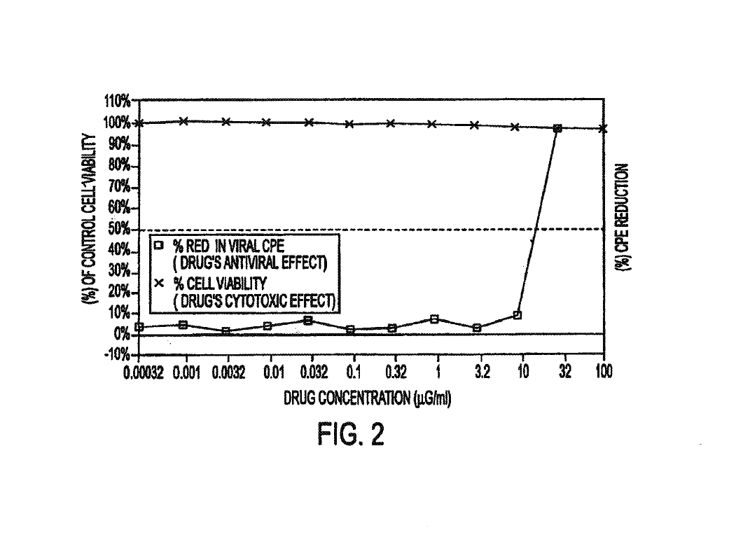

FIG. 3 is a graph depicting the antiviral activity of a prior art compound.

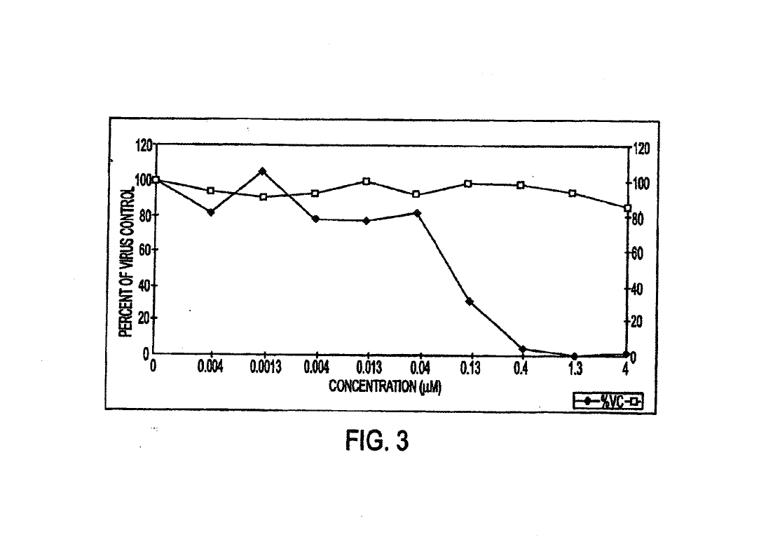

FIG. 4 is a graph depicting the antiviral activity of a compound of the present invention.

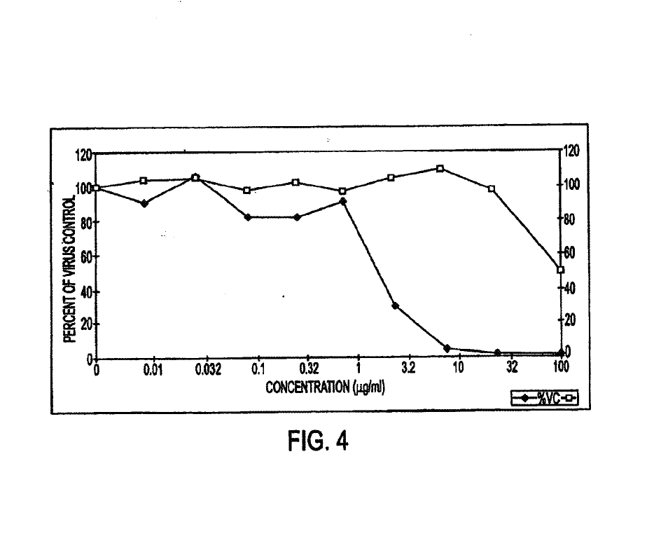

FIG. 5 shows the susceptibility of Bacillus anthracis strains against BVS-10A and Ciprofloxacin.

FIG. 6 shows the susceptibility of Burkholderia pseudomallei strains against BVS-10A and Ceftazidime.

FIG. 7 shows the susceptibility of Burkholderia mallei strains against BVS-10A and Azithromycin.

FIG. 8 shows the susceptibility of Francisella tularensis strains against BVS-10A and Ciprofloxacin.

FIG. 9 shows the susceptibility of Yersinia pestis strains against BVS-10A and Ciprofloxacin.

FIG. 10 shows the dose response graphs for Mycobacterium tuberculosis H37Rv using BVS-10A.

FIG. 11 shows the dose response graphs for Mycobacterium tuberculosis Isoniazid Resistance using BVS-10A.

FIG. 12 shows the dose response graphs for Mycobacterium tuberculosis Rifampin Resistance using BVS-10A.

FIG. 13 shows the dose response graphs for Mycobacterium tuberculosis Ofloxacin Resistance using BVS-10A.

FIG. 14 shows the Results for the LORA assay for Mycobacterium tuberculosis using BVS-10A.

FIG. 15 shows the intracellular BVS-10A activity reported as log reduction values calculated as reduction in Mycobacterium tuberculosis concentration from zero hour to 7 days post-infection.

FIG. 16 is a survival graph of treated and untreated mice infected with 10 LDSO Yersinia pestis.

FIG. 17 is a survival graph of treated and untreated mice infected with 3 LD₅₀ F. tularensis.

### DETAILED DESCRIPTION OF THE INVENTION

It has now been discovered that the compounds of this invention have dual mechanism of anti-viral and antibacterial action.

In one embodiment, the compound known as “BVS-10A” having the formula:

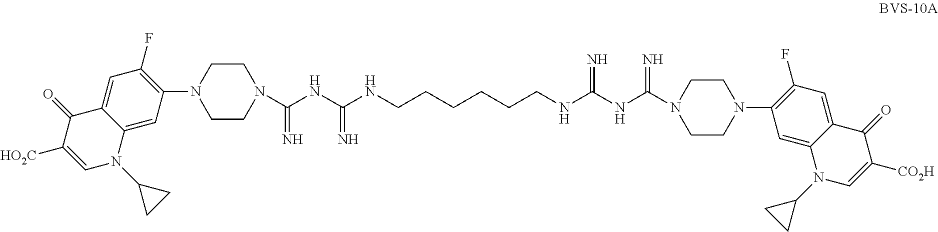 [SMILES: N=C(NCCCCCCNC(=N)NC(=N)N1CCN(c2cc3c(cc2F)c(=O)c(C(=O)O)cn3C2CC2)CC1)NC(=N)N1CCN(c2cc3c(cc2F)c(=O)c(C(=O)O)cn3C2CC2)CC1]

was found to have dual anti-HIV and antibacterial activity. The compound and its derivatives have the advantage of treating not only humans identified as having AIDS as well as humans identified as infected by or carrying the AIDS virus to prevent or inhibit the acquired immunodeficiency syndrome, but also to prevent or inhibit opportunistic infections. It is recognized that in AIDS patients, there is an imbalance in the two basic types of immune system cells, namely helper/inducer T lymphocytes, with the ratio of suppressor cells to helper/inducer cells greatly elevated. It was shown that the imbalance is caused by depletion of the helper/inducer T cells. When the patient is treated with the “BVS-10” compound or its derivatives, it is possible that the number of helper T cells, T cells and platelets may increase over those before treatment.

The bifunctional compounds of the present invention exhibit a broadened antiviral and antibacterial spectrum of activity reflecting both quinolone, azaquinoline, (aza)quinolone-cyclam, (aza)quinolone-bicyclam and biguanidine or guanidine contribution, and suggestive of a dual mode of action. The beneficial influence of quaternary nitrogen substituents on the biological activity has been noted by the potency of the molecule against both AIDS virus and bacteria. The activity may involve the transportation, RNA and intracellular inactivation and inhibition of the essential enzymes reverse transcriptase and DNA gyrase. The invention relates to a method of treating AIDS virus in warm-blooded animals with the antiviral composition or antibacterial composition containing the compounds of the invention. More particularly, the invention comprehends an antiviral composition containing the new compounds and at least one pharmaceutically acceptable carrier used to treat warm blooded animals infected with (or to prevent infection with) viruses and bacteria, particularly the AIDS virus known as human immunodeficiency virus (HIV).

The present invention relates to a new class of compounds containing both hydrophilic and lipophilic regions. The basic structure may be represented by the general formulae given above, where the various substituents are as defined.

In certain preferred embodiments, the “A” substituent is a quinolone or azaquinolone antibacterial moiety such as:

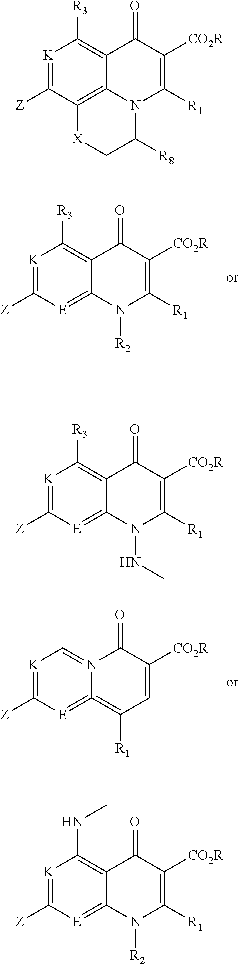

where Z is one or more heterocyclic rings containing at least one N atom
or

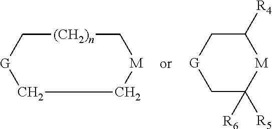

which may be attached to the structure above through any available point of attachment;

where n is 0 to 3; R₄, R₅ and R₆ are each independently hydrogen or lower alkyl or alkylene; G and M are independently O, S or NR₁₀; where R₁₀ is hydrogen, —C(N)—NH—CN, halogen, a single bond, or lower alkyl or alkylene;

E is nitrogen or CR₁₀, where R₁₀ is hydrogen, halogen, lower alkoxy optionally substituted with one or more halogens or lower alkyl optionally substituted with one or more halogens;

K is nitrogen or CR₇, where R₇ is hydrogen, nitro, halogen, nitrile, carboxamide, carboxyl or an ester;

R is hydrogen or lower alkyl;

R₁ is hydrogen, a lower arylalkyl group having 1-6 carbon atoms, an alkyl group having 1-4 carbon atoms in the aliphatic part and 6 to 10 carbon atoms in the aromatic part, or an aryl group having 6 to 10 carbon atoms;

R₂ is an alkyl group having one to six carbon atoms; a cycloalkyl group having 3 to 7 carbon atoms optionally substituted with halogen; 2,4-difluorophenyl or 2- or 4-fluorophenyl; amino; lower alkylamino; propylamino; N-formyl-lower alkylamino or di-lower-alkylamino; a vinyl group; a 2-fluoroethyl group; a haloalkyl group or a 2-hydroxylkyl group; phenyl or substituted phenyl wherein the phenyl ring is substituted with one or two or three substituents independently selected from C₁ to C₆ alkyl, halogen, methylenedioxy and hydroxy; alkoxy or trifluoromethyl; 2-, 3-, or 4-pyridine; 2- or 3-thiophene; 2-imidazole; 2-oxazole or 2-thiazole; a pyridyl or adamantyl group; or a benzoxazine group;

R₃ is a hydrogen, amino, substituted amino, halogen or a lower alkyl group;

R₈ is hydrogen, lower alkyl optionally substituted with one or more halogen atoms, or lower alkoxy optionally substituted with one or more halogen atoms; and X is methylene, O, S or NR₉, where R₉ is hydrogen or lower alkyl optionally substituted with one or more halogen atoms, or lower alkoxy optionally substituted with one or more halogen atoms.

More specifically, the Z substituent may include

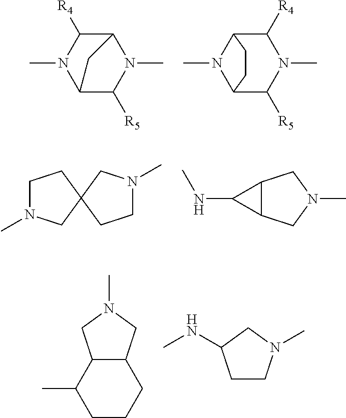

where R₄ and R₅ are independently hydrogen or lower alkyl.

It will be readily appreciated from the structures above that certain compounds of the present invention may exist as optically active forms. The pure D-isomer, pure L-isomer as well as mixtures thereof, including the racemic mixtures, are contemplated by the invention. Additional asymmetric carbon atoms may be present in a substituent such as in an alkyl group, piperazine or benzoxazine moiety. All such isomers as well as mixtures thereof are intended to be included in the invention.

The term haloalkyl group under the definition of R₂ above is intended to include halogen substituted straight and branched carbon chains of two to six carbon atoms. Those skilled in the art will recognize that the halogen substituent may not be present on the 2-carbon atom of the chain. Representative of such groups are β-chloroethyl, β-dichloroethyl, β-chloropropyl, β-chloro-2-propyl, α-iodobutyl, β-fluoroethyl, β-difluoroethyl, and the like the term halogen is intended to include, fluorine, chlorine, bromine and iodine unless otherwise specified.

The present invention also includes the salts of the compounds described above. Such salts may be derived from inorganic acids such as hydrochloric acid, or phosphoric acid; from organic acids such as acetic acid, lactic acid, oxalic acid, succinic acid, methane sulfonic acid, maleic acid, malonic acid or gluconic acid; from acidic amino acids such as aspartic acid or glutamic acid; metal (e.g. sodium, potassium, calcium, magnesium or zinc) salts; from organic bases such as N,N-dibenzyl ethylene diamine, dimethylamine, triethylamine, dicyclohexylamine, benzylamine or ethylene diamine, and from basic amino acids such as lysine or arginine.

The esters of the compound of the formula represented by A include not only substituted or unsubstituted aliphatic esters especially lower alkyl esters such as methyl or ethyl esters but also esters that can be at least partially converted to the compound A of structure I, II, III or IV or V by chemical hydrolysis or by enzymatic hydrolysis in vivo such as acetoxymethyl esters, pivaloyloxymethyl esters, ethoxycarbonyloxy ethyl esters, choline esters, aminoethyl esters (e.g. dimethylaminoethyl or 1-piperidinylethylesters), 5-indanyl esters, phthalidyl esters and hydroxyalkyl esters (e.g. hydroxyethyl or 2,3-dihydroxypropyl esters).

The term “lower” means that the groups or compounds so qualified have not more than 6, preferably not more than 4 carbon atoms. The compounds of the specific structures shown above, and esters and salts of these compounds will, therefore, be generally referred to herein as the compounds of the invention. The compounds of the invention may also exist as hydrates, clathrates, which are also included in the compounds of this invention. The compounds of the invention include those, which have asymmetric carbon atoms on the piperazine, pyrrolidine or homopiperazine ring at the 7-position or at 1,4-benzoxazine ring or at the dimeric alkyl chain interposed between the two biguanidine chains and therefore exists in optically active forms. Hence, D-isomers, L-isomers and mixtures thereof all included in the compounds of this invention. The compounds of this invention may have two asymmetric carbon atoms simultaneously and therefore can exist as stereoisomers having different configurations (cis or trans form). These stereoisomers and their mixtures are also included within the compounds of this invention.

As noted above, compounds within the present invention have antiviral and antibacterial activity and thus may be administered to patients in need thereof. For therapeutic or prophylactic treatment, the compounds of the present invention may be formulated in a pharmaceutical composition, which may include, in addition to an effective amount of active ingredient, pharmaceutically acceptable carriers, thickeners, diluents, buffers, preservatives, to surface active agents and the like. Pharmaceutical compositions may also include one or more other active ingredients if necessary or desirable.

The treatment method may include treatment of a subject infected with one or more virii or bacteria, including, but not limited to Yersinia pestis, Bacillus anthracis, Francisella tularensis, Burkholderia pseudomallei, and Burkholderia mallei, Escherichia coli, Pseudomonas aeruginosa, Plasmodium falciparum, Mycobacterium tuberculosis, influenza A H3N2, influenza A H1N1, HIV, or a combination thereof.

The pharmaceutical compositions of the present invention may be administered in a number of ways as will be apparent to one of ordinary skill. Administration may be done topically, orally, rectally, nasally, vaginally, by inhalation, or parenterally (including subcutaneous, intramuscular, intravenous and intradermal), for example.

Topical formulations may include ointments, lotions, creams, gels, drops, suppositories, sprays, liquids and powders. Oral formulations include powders, granules, suspensions or solutions in water or non-aqueous media, capsules or tablets, for example. Thickeners, flavorings, diluents, emulsifiers, dispersing aids or binders may be used as needed.

Parenteral formulations may include sterile aqueous solutions which may also contain buffers, diluents and other suitable additives.

The dose regimen will depend on a number of factors which may readily be determined, such as severity and responsiveness of the condition to be treated, but will normally be one or more doses per day, with a course of treatment lasting from several days to several months, or until a cure is effected or a diminution of disease state is achieved. One of ordinary skill may readily determine optimum dosages, dosing methodologies and repetition rates. In general, it is contemplated that unit dosage form compositions according to the present invention will contain from about 0.01 mg to about 500 mg of active ingredient, preferably about 0.1 mg to about 10 mg of active ingredient. Topical formulations (such as creams, lotions, solutions, etc.) may have a concentration of active ingredient of from about 0.1% to about 50%, preferably from about 0.1% to about 10%. However, final strength of the finished dosage form will depend on the factors listed above and may be readily determined by one of ordinary skill.

In certain embodiments, for example for treating plague, about 500 mg of active ingredient may be administered. In such an embodiment, the formulation may be administered by any suitable administration route, for example orally and/or intravenously.

In certain embodiments, for example for treating community acquired pneumonia, about 500 to about 750 mg of active ingredient may be administered. In such an embodiment, the formulation may be administered by any suitable administration route, for example orally and/or to intravenously.

In certain embodiments, for example for treating acute bacterial sinusitis, about 500 to about 750 mg of active ingredient may be administered. In such an embodiment, the formulation may be administered by any suitable administration route, for example orally and/or intravenously.

In certain embodiments, for example for treating uncomplicated skin/skin structure infections, about 500 mg of active ingredient may be administered. In such an embodiment, the formulation may be administered by any suitable administration route, for example orally and/or intravenously.

The compounds of the present invention may be synthesized according to the following general reaction schemes:

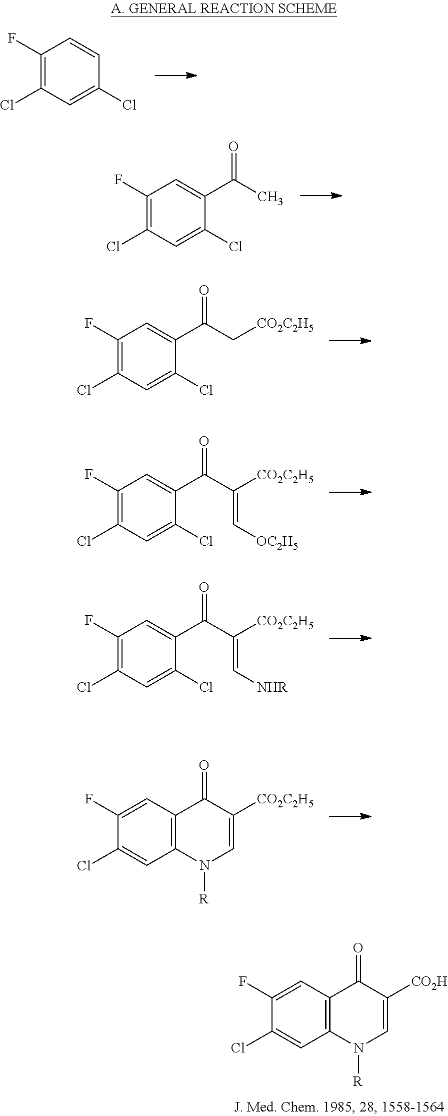

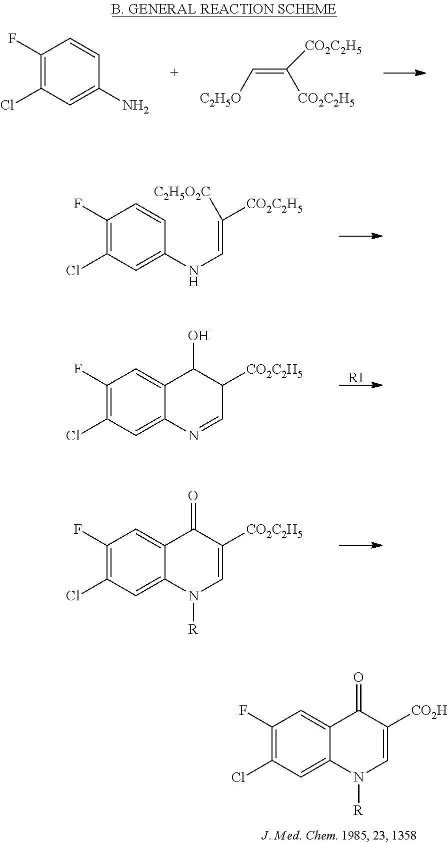

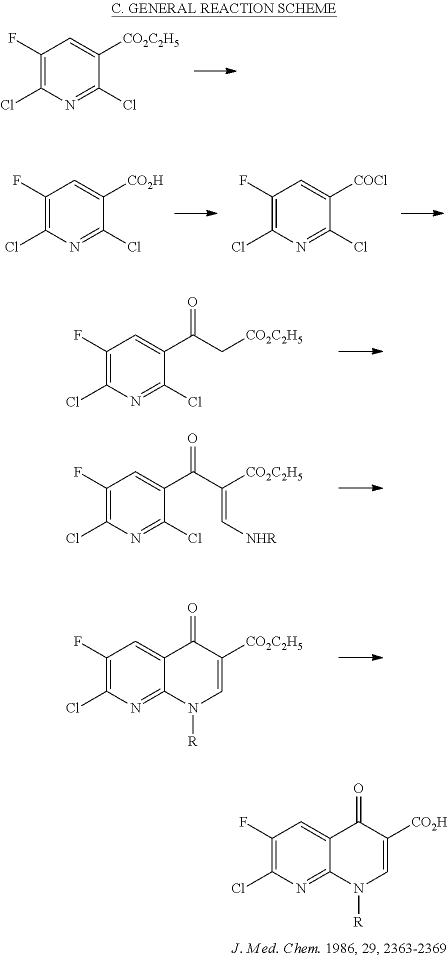

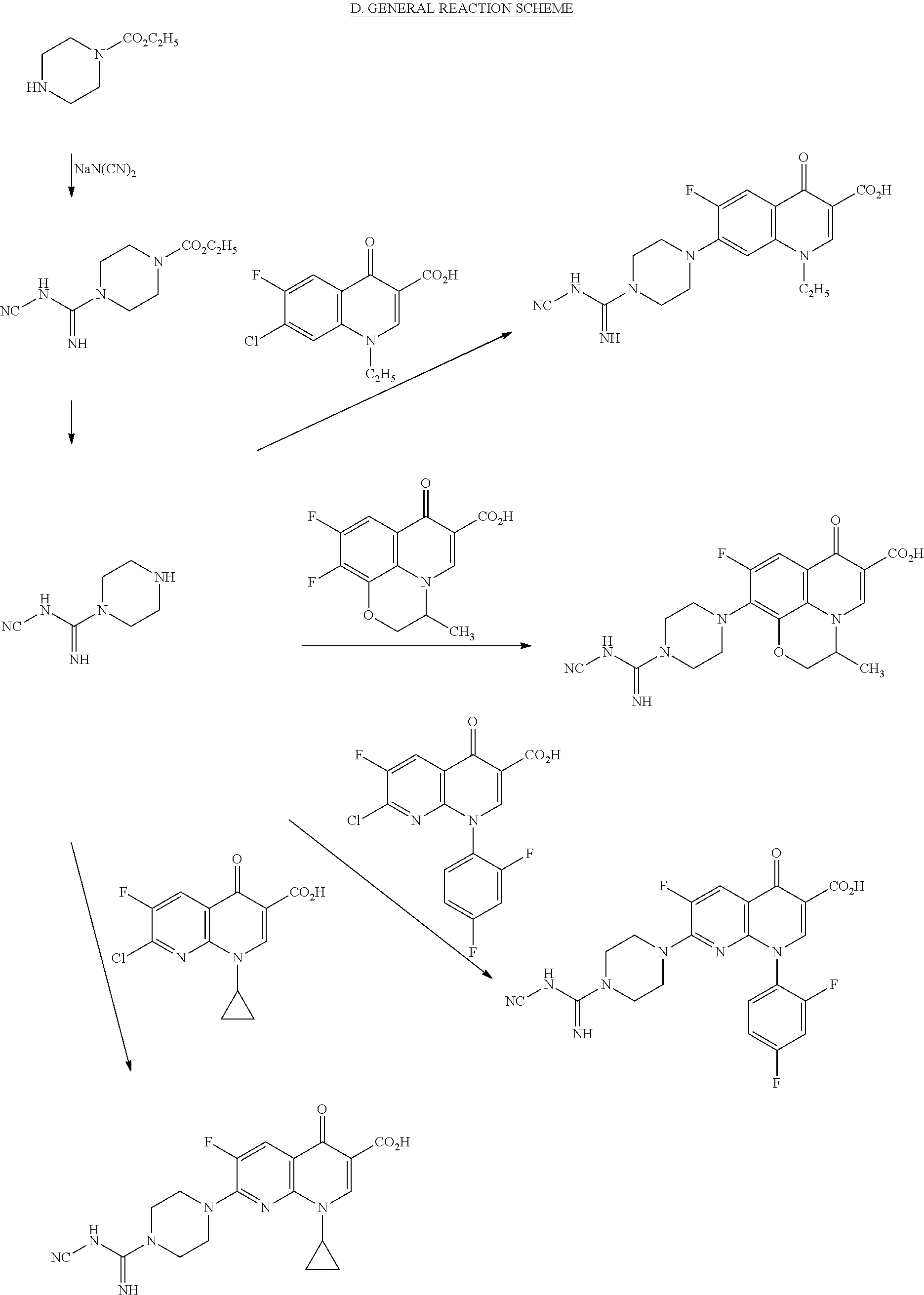

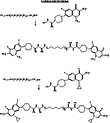

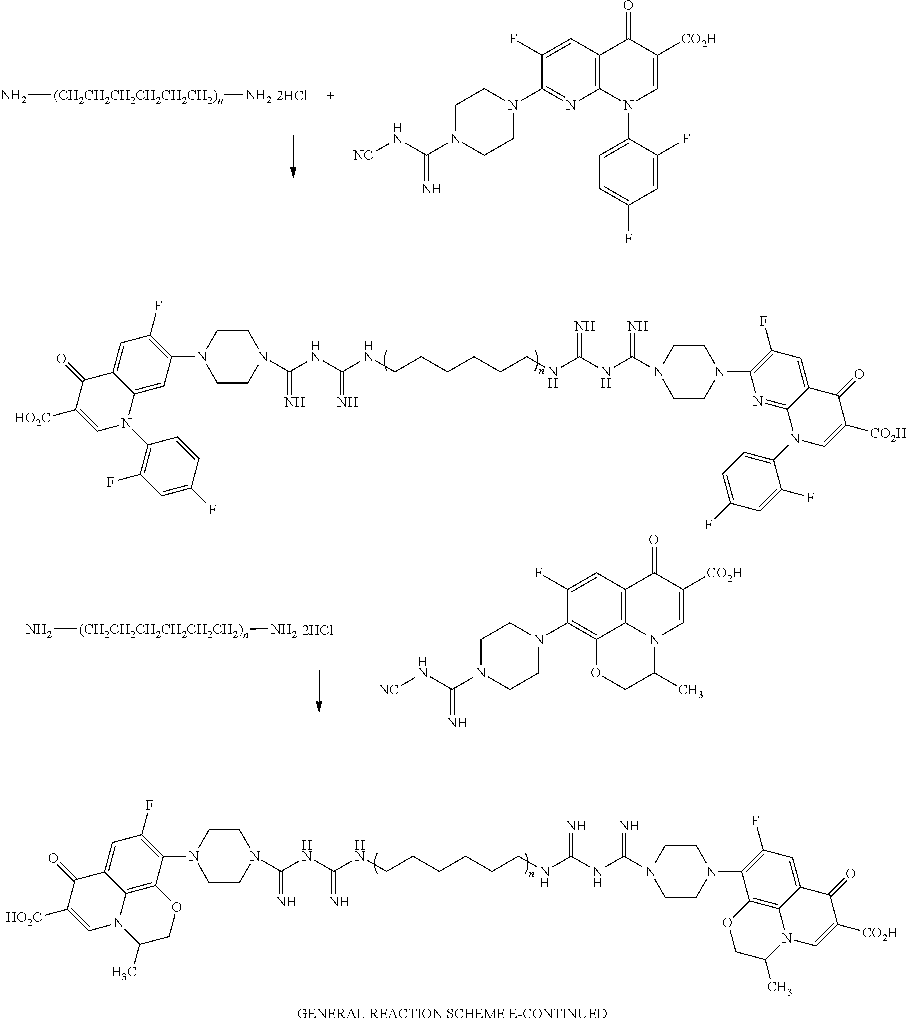

The compounds of this invention of the formula (I), (II), (III) or (IV) can be prepared by various alternative procedures. Some of the starting compounds in the reaction scheme are known in the literature. They have been noted. Following are some specific reaction schemes for their preparation.

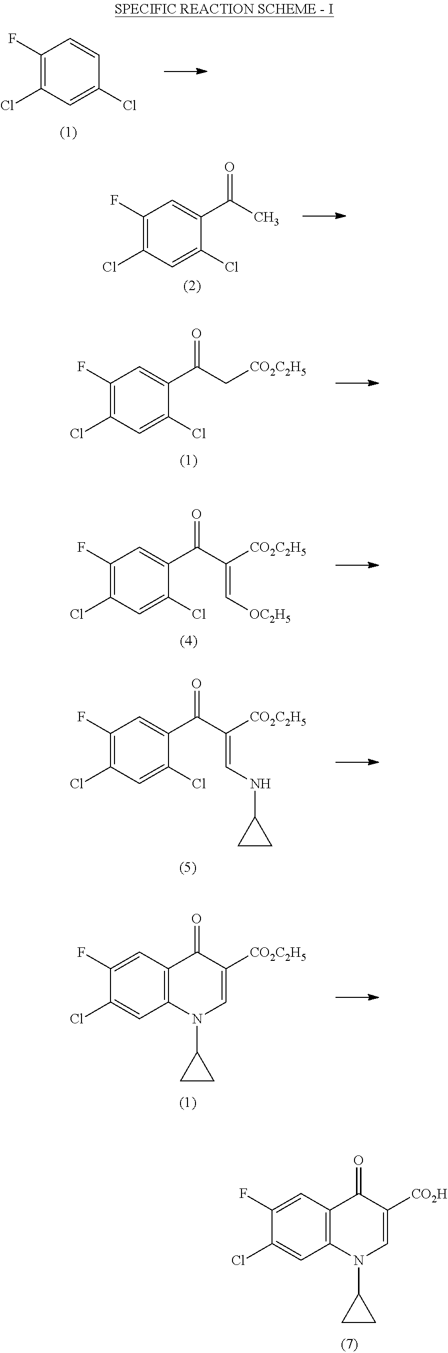

### 2,4-Dichloro-5-fluoroacetophenone (2)

To a warm solution of 1,3-dichloro-4-fluorobenzene at 70° C. (175.0 gm), anhydrous aluminum chloride was added in small portion with stirring (260.0 g). Then acetic anhydride was added in small portion with stirring (125 mL) followed by an additional amount of aluminum chloride (159.0 g). The temperature was slowly raised to 120° C. with stirring for 25 hours. The thick reaction mixture was cooled and poured into 400 cc of concentrated hydrochloric acid containing ice. The oil was extracted with methylene chloride, washed with water, dried over anhydrous magnesium sulfate and concentrated to give 165.0 g of oil (2).

### Ethyl 2,4-Dichloro-5-fluorobenzoyl acetate (3)

To a mixture of 60% sodium hydride dispersion powder (66.0 g) in tetrahydro furan (330 mL) under a blanket of nitrogen, diethyl carbonate was added (680 mL). The mixture was heated to 45° C. with stirring and a solution of 2,4-dichloro-5-fluoro acetophenone (2) (165.0 g) in diethyl carbonate (130 mL) was added to it maintaining the temperature between 50° C.-55° C. for 5 hours. It was concentrated under reduced pressure, mixed with dichloromethane (500 mL) and treated with glacial acetic acid (240 mL) in water (2.7 L). The combined methylene chloride extract was washed with sodium bicarbonate (66.0 g) in water (1 L). It was dried over anhydrous magnesium sulfate and concentrated to give 215.0 g oil (3). It was used for next step without further purification.

### 2,4-Dichloro-alpha(elhoxymethlene)-5-fluoro-beta-oxo-benzene propanoic acid ethyl ester (4)

A mixture of triethyl orthoformate (209 mL), acetic anhydride (350 mL) and (3) (215.0 g) was refluxed with stirring under nitrogen for 6 hours while collecting most of ethyl acetate. The solution was concentrated to give 226.0 g. of oil (4).

### 7-Chloro-6-fluoro-1-cyclopropyl-1,4-dihydro-4-oxo-quinoline-3-carboxylic acid (7)

To a solution of 4 (80 g) in methylene chloride (800 mL), cyclopropylamine (44.0 g) was added with cooling below 10° C. The mixture was stirred at room temperature for 1 h and evaporated to a dry mixture. To the dry mixture in dimethoxyethane (800 mL), a 60% sodium hydride-in-oil suspension (12.0 g) was added slowly with cooling and stirring. The mixture was heated at 80-85° C. for 3 h under nitrogen atmosphere. It was cooled and water (5 L) was added and the precipitate was filtered and washed with water and dried. The ester was suspended in tetrahydrofuran (800 mL). A solution of sodium hydroxide (20.0 g) in water (500 mL) was added and the mixture was refluxed for 2 h. It was cooled and acidified with acetic acid and the precipitate was filtered. The solid was washed with water and acetone. wt=19.0 g of (7). m.p.=235-240° C.

Anal. calcd. for: C₁₃H₉NFClO₃. Theory: C, 55.43; H, 3.22; N, 4.97; F, 6.77; Cl, 12.58. Found: C, 55.42; H, 3.26; N, 4.95; F, 5.92; Cl, 12.31.

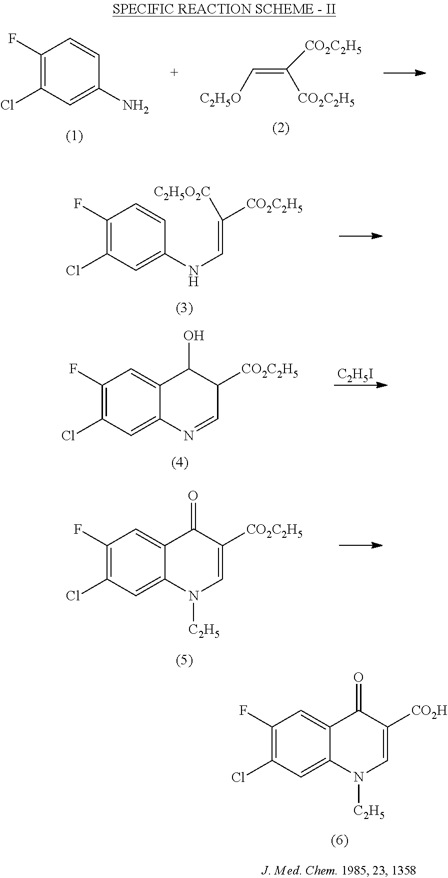

### 7-Chloro-6-fluoro-4-hydroxyquinoline-3-carboxylic acid ethyl ester (4)

A mixture of 3-chloro-4-fluoroaniline (1) (87.3 g) and diethyl ethoxymethylene malonate (129.7 g) and Dowtherm (700 mL) (diphenyl ether) was refluxed for 4 h. It was cooled and the resulting solid was removed by filtration. The solid was recrystallized from DMF to give 55.0 g (4). m.p.=>300° C.

Anal. Calcd for C₁₂H₉NFClO₃

| | C | H | N | F | Cl |
|---|---|---|---|---|---|
| Theory: | 53.45 | 3.36 | 5.19 | 7.04 | 13.14 |
| Found: | 53.45 | 3.48 | 5.26 | 6.53 | 12.67 |

### 7-Chloro-1-ethyl-6-fluoro-1,4-dihydro-4-oxoquinoline-3-carboxylic acid (6)

A mixture of 7-chloro-6-fluoro-4-hydroxyquinoline-3-carboxylic acid ethyl ester (4) (55.0 g), anhydrous potassium carbonate (8.0 g) and DMF (150 mL) was heated with stirring at 125° C. for 3 h. It was cooled to 70° C. and ethyl iodide (19.0 g) was added and continued heating at 120° C. for 4 h. The mixture was evaporated to dryness, extracted, with CH₂Cl₂, washed with water, dried and evaporated to dryness. The crude ester was recrystallized from EtOH to yield (5) m.p.=142-143° C. wt=50.0 g. The ester (5) was mixed with 2N NaOH (700 mL) and refluxed with stirring for 2 h. It was cooled acidified with AcOH and the resulting precipitate was filtered off, washed with water and dried. The solid was recrystallized from DMF to yield (6)=18.0 g., m.p. 284-285° C.

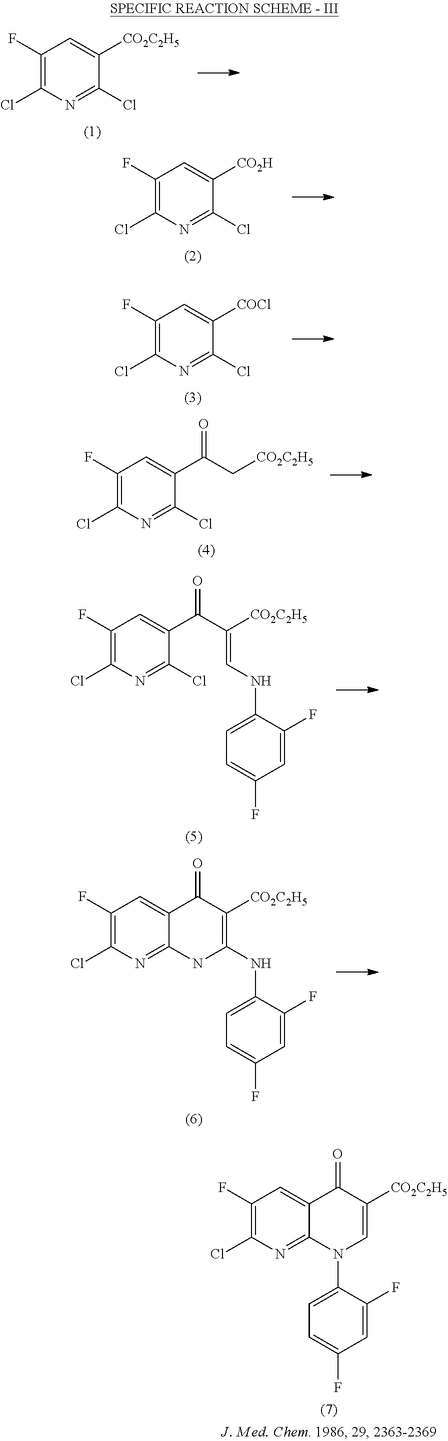

### 2,6-Dichloro-5-fluoronicotinic acid (2)

Ethyl 2,6-dichloro-5-fluoronicotinate (1) (30.0 g) was dissolved in a mixture of 60 mL of trifluoro acetic acid and 60 mL of 7.5 N HCl. The mixture was refluxed with stirring for 26 h. It was cooled and the trifluoroacetic acid was removed under reduced pressure. The solution was mixed with 150 mL water, the resulting precipitate was filtered, washed with hexane and dried to yield 16.5 g of

2,6-dichloro-5-fluoro nicotinic acid (2). m.p.=152-156° C.

### Ethyl 2,6-dichloro-5-fluoronicotinylacetate (4)

2,6-Dichloro-5-fluoronicotinic acid (2) (14 g) was dissolved in thionyl chloride (70 mL). The mixture was heated at 85° C. with stirring for 2.5 h and the thionyl chloride was removed under reduced pressure, yielding a yellowish oil, 2,6-dichloro-5-fluoronicotinyl chloride (3). Monoethyl malonate (27 g) and 6 mg of biquinoline was dissolved in 560 mL of dry THF and cooled to −30° C. A solution of 2.5 M of n-butyllithium in hexane was added until a pink color remained at −5° C. (160 mL). The suspension was then cooled to −50° C. The acid chloride obtained as described above, dissolved in 50 mL THF was then added to the suspension dropwise with stirring. The dry ice bath was removed and the reaction mixture was stirred at room temperature for 1 h. The reaction was acidified with 400 mL of 1 N HCl and was extracted with ether. It was washed with aqueous sodium bicarbonate solution and water. The ether solution was dried, evaporated to dryness and washed with hexane to give 27 g of (4). m.p=63-66° C.

Ethyl-3,1-(2,4-Difluoroanilino)-2-(2,6-dichloro-5-fluoronicotinyl) acrylate (5).

A solution of ethyl 2,6-dichloro-5-fluoro nicotinylacetate (4) (8 g) in triethyl orthoformate (7 mL) and acetic anhydride (50 mL) was heated at 130° C. for 1 h with removal of ethyl acetate formed during the reaction. The solution was evaporated under reduced pressure to give mobile oil. The oil was dissolved in methylene chloride (250 mL) and 2,4-difluoroamline (5.2 g) was added to the solution. After standing for 1 h, the solution was evaporated to dryness and the residue recrystallized and washed with hexane to give 9 g of (5). m.p.=138-141° C.

### Ethyl 1-(2,4-difluoroanilino)-6-fluoro-7-chloro-1,4-dihydro-4-oxo-1,8-naphthyridine-3-carboxylate (6)

A 60% sodium hydride in oil suspension (1.73 g) was slowly added with stirring to a cold solution of nicotinylacrylate (5) (16.5 g) in THF (200 mL). The mixture was refluxed with stirring under nitrogen for 1.5 h, cooled, washed with water and dried yielding 8 g of (6). m.p.=210-212° C.

### 1-(2,4-Difluoroanitino)-6-fluoro-7-chloro-1,4-dihydro-4-oxo-18-naphthylidine-3-carboxylic acid (7)

A suspension of (6) (10 g) in 29 mL of 12% HCl and 26 mL of glacial acetic acid was refluxed for 7.5 h. It was cooled, filtered and recrystallized from a boiling mixture of methanol and acetone to yield 6.3 g of (7) m.p.=195-200° C.

Anal. Calcd. for: C₁₅H₆N₂F₃ClO₃

| | C | H | N | F | Cl |
|---|---|---|---|---|---|
| Theory: | 50.80 | 1.70 | 7.89 | 16.06 | 9.99 |
| Found: | 51.86 | 2.64 | 7.33 | 15.31 | 9.47 |

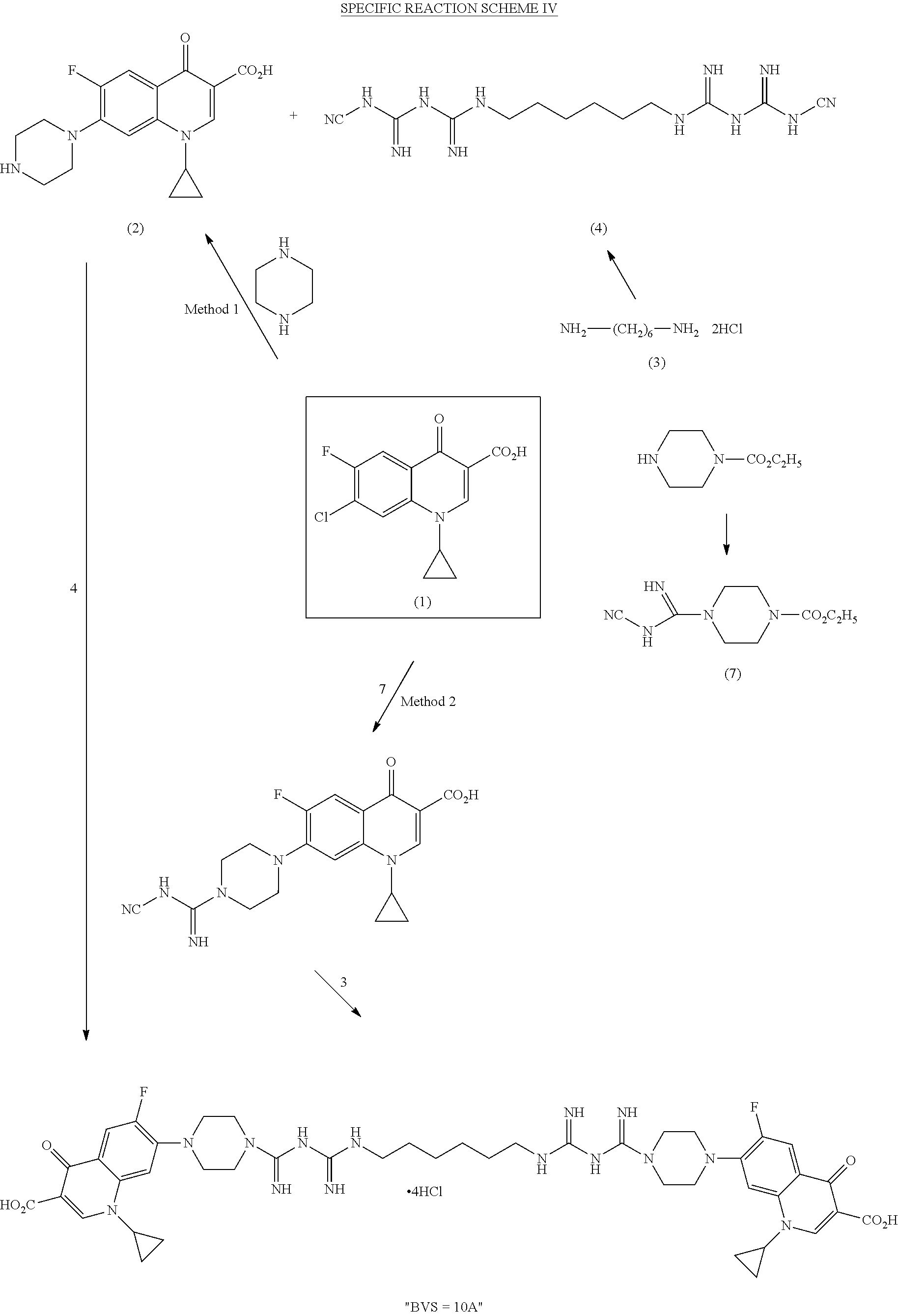

### 1-Cyclopropyl-6-fluoro-1,4-dihydro-4-oxo-7-(1-piperazinyl)-quinoline carboxylic acid monohydrochloride monohydrate (2)

### Method-1

7-Chloro-6-fluoro-1-cyclopropyl-1,4-dihydro-4-oxo-quinoline-3-carboxylic acid (1) (10 g) was added with stirring to melted piperazine (10 g) at 155-160 [symbol] C during 15 min. heating was continued for 0.5 h. The reaction mixture was cooled, mixed with water and filtered. The resulting solid was mixed with water and ethanol, acidified with concentrated HCl, boiled and filtered. To the clear filtrate, activated charcoal was added, boiled and filtered. It was cooled on dry ice and the solid was removed by filtration to yield 7.8 g (2). m.p. 317-321° C.

### Hexamethylene bis(cyanoguanidine) (4)

1,6-Hexamethylene diamine dihydrochloride (20 g), sodium dicyandiamide (18.8 g), and 1-butanol (250 mL) were mixed and refluxed with stirring 15 h. The reaction mixture was cooled, filtered and washed with acetone. The resulting solid was mixed with water (200 mL), acidified with HCl, filtered and washed with water. It was mixed with water again, made basic NaOH, filtered, washed with water, followed by acetone and dried. Wt. 14 g (4). m. p.=195-200° C.

A mixture of (2) (5 g), (4) (1.7 g) and n-butanol (150 mL) was refluxed with stirring for 76.5 h. It was evaporated under reduced pressure. The solid was mixed with water, acidified with cone HCl, mixed with charcoal, boiled and filtered. It was cooled, precipitated with acetone, filtered and the resulting solid was recrystallized again from boiling methanol to yield 1.5 g of “BVS-10A”. m.p. 100-105° C.

Anal. Calcd. for: C₄₄H₅₈Cl₄F₂N₁₄O₆H₂O

| | C | H | Cl | F | N |
|---|---|---|---|---|---|
| Theory: | 49.07 | 5.61 | 13.69 | 3.52 | 18.21 |
| Found: | 48.66 | 5.94 | 12.38 | 3.62 | 18.28 |

In one embodiment, the invention includes BVS-10A or a salt thereof in combination with phosphanilic acid. The combination of BVS-10A and phosphanilic acid further enhances the bactericidal activity of these components especially against Proteus, Providencia and Pseudomonas species.

### Anti-HIV Test Data OF “BVS-10A”.

Anti-HIV Test Data and the Methods Used to Test the Compound, BVS-10A.

The compound was evaluated by two test methods to establish anti-HIV (human immunodeficiency virus) activity of BVS-10A.

### Method 1: CEM-SS Assay Method (SS Stands for Syncytium Sensitive)—Description:

This is a cytoprotection assay that uses the T-lymphoblastoid tumor cell line CEM-SS and laboratory adapted strains of HIV-1. Upon infection with HIV, the CEM-SS cells form syncytia and eventually die, producing large amounts of progeny virus over the course of 4-6 days at the standard multiplicity of infection (0.01). The antiviral assay measures the ability of a Liven compound to inhibit the HIV-induced cell killing. In order to provide the best opportunity for the compound to demonstrate anti-HIV activity, the compound is added to uninfected cells in the microtiter plate well shortly before the addition of cell-free infectious virus. The activity of the compound is evaluated by measuring cell viability at 6 days post-infection and comparing the viability to that obtained in cell control (cells only), virus control (cells and virus only) and toxicity control (cells and compound only) wells. In general it is a very effective assay for the detection of anti-HIV activity in vitro and is predictive of anti-HIV assay in more clinically relevant assays with compound possessing a wide range of mechanism of action.

### Method 2: PBMC Assay Method (Peripheral Blood Mononuclear Cell)—Description

This assay addresses the issue of providing a rationale for further evaluation of the compound by providing evidence of in vitro anti-HIV activity against a low passage clinical strain of HIV-1. These fresh human peripheral blood mononuclear cell assays use fresh human cells obtained from the American Red Cross from uninfected, normal human donors. Mononuclear cells from these blood samples are isolated by centrifugation and infected with low passage clinical strains of HIV-1 which have been obtained by culture of blood cells from HIV-infected patients with uninfected PBMCs. Since the PBMCs are not killed by HIV infection, endpoint quantification is performed by measurement of virus production in each well by reverse transcriptase assay or p24 ELISA. These assays provide evidence that the test compounds will have efficacy against viruses, which will likely be encountered in patients without genetic changes, introduced by passage in cell culture.

### DEFINITIONS

Efficacy of the compound in cell culture is measured by increased cell viability in CEM-SS assays or reduction in virus production in PBMC assays. The data obtained yields a dose response curve which can be evaluated to determine doses which yield 25%, 50% and 95% protection from HIV-induced cell killing or virus production. These concentration are reported as the IC25, IC50 and IC95 values. Similarly since toxicity is evaluated in parallel, the 25%, 50% and 95% toxic concentrations can be calculated based on the reduction of cell viability observed in the presence of drug alone. These concentrations are reported as the TC25, TC50 and TC95 values. The ratio of toxicity to efficacy constitutes the therapeutic index TC50/IC50.

### Data:

BVS-10A was active at two concentrations and provided 100% protection at 32 and 100 μg/ml, in CEM-SS cells. The calculated IC₅₀ was 16.6 μg/mL and the IC₉₅ was 30 μg/mL. The observed therapeutic index was not calculated since the compound remained nontoxic at 100 μg/mL (T1>10). BVS-10A was nontoxic at 100 μg/mL. In the microliter XTT assay, protection from HIV-induced cell killing in the CEM-SS cells is directly related to a reduction in virus load as measured by RT (reverse transcriptase) or p24 ELISA. Complete protection in this assay therefore is correlated with complete suppression of virus production.

BVS-10A was determined to be active at concentrations greater than 3.2 μg/mL in PBMCs. Toxicity was detected in PBMCs at 100 μg/mL and no toxicity was observed in CEM-SS cells at concentrations up to 100 μg/mL. The compound, BVS-10A becomes 100% active in both anti-HIV assays performed. Though virus production in CEM-SS cells is not directly measured, in general, protection from HIV-induced cell killing usually correlates directly with reduced virus load. Thus, at points where 100% protection is observed, it is expected that little to no virus production at those concentrations. In the PBMC assays, the end point used does measure the amount of virus produced from the infected, treated cells and in this assay, 100% protection does mean complete suppression of virus production.

The compound, BVS-10A was tested against HIV using di-deoxycytosine (ddC) as a positive control drug by CEM-SS cell method. The test results are shown in Tables 1 and 2. It was also tested against HIV using AZT as a positive control by PBMC method. The test results are given in Tables 3 and 4. RT stands for reverse transcriptase.

TABLE 1

| | PLATE HAS | | | IN VITRO ANTIVIRAL RESULTS | | | | | DRUG: DDC | | | |
|---|---|---|---|---|---|---|---|---|---|---|---|---|
| | DRUG DOC | | | XTT ASSAY | | | | | SI: >485.51 | | | |
| | 1 | 2 | 3 | 4 | 5 | 6 | 7 | 8 | 9 | 10 | 11 | 12 |
| | reagent background | | | | | | plastic background | | | | | |
| A | 0.187 | 0.179 | 0.180 | 0.178 | 0.183 | 0.184 | 0.088 | 0.086 | 0.086 | 0.086 | 0.082 | 0.083 |
| | lox | cc/vc | experimental = high | | | lox | lox | experimental = low | | | cc/vc | lox |
| | | | concentration | | | | | concentrations | | | | |
| B | 1.902 | 1.648 | 1.329 | 1.695 | 0.846 | 1.772 | 1.688 | 0.290 | 0.277 | 0.325 | 1.755 | 1.688 |
| C | 1.731 | 1.572 | 1.316 | 1.530 | 1.273 | 1.418 | 1.460 | 0.265 | 0.440 | 0.281 | 1.645 | 1.584 |
| D | 1.719 | 1.560 | 1.840 | 1.324 | 1.482 | 1.504 | 1.594 | 0.386 | 0.318 | 0.342 | 1.579 | 1.617 |
| E | 1.818 | 0.380 | 1.500 | 1.458 | 1.369 | 1.490 | 1.537 | 0.286 | 0.323 | 0.380 | 0.238 | 1.779 |
| F | 2.231 | 0.251 | 2.096 | 1.981 | 1.997 | 1.791 | 1.519 | 0.429 | 0.392 | 0.317 | 0.311 | 1.738 |
| G | 2.538 | 0.275 | 1.946 | 1.922 | 2.020 | 1.959 | 1.729 | 0.456 | 0.310 | 0.395 | 0.244 | 1.724 |
| | colorimetric background = | | | | | | colorimetric background = | | | | | |
| | high concentration | | | | | | low concentrations | | | | | |
| H | 0.185 | 0.180 | 0.185 | 0.212 | 0.184 | 0.179 | 0.177 | 0.179 | 0.182 | 0.178 | 0.176 | 0.192 |

lox = cell loxicity
cc = cell control
vc = virus control
BOLD = highest drug conc
values shown are optional densities

| | | | | |
|---|---|---|---|---|
| VIRUS | HIV1 | PASSAGE - | PROJECT # | — |
| CELLS | CEMSS | PASSAGE 15 | SPONSOR | SHETTY |
| | | OPERATOR 12S | TEST DATE | Sep. 10, 1997 |
| | | | DATE READ | Sep. 16, 1997 |

| STRN | RF | DRUG DDC | 25% | 50% | 95% |
|---|---|---|---|---|---|
| REAGENT | 0.182 | TC (CM) | >10.00 | >10.00 | >10.00 |
| VIRUS CONTROL | 0.091 | IC (CM) | 0.01 | 0.02 | 2.11 |
| CELL CONTROL | 1.445 | ANTIVIRAL INDEX (AI) | >754.29 | >485.51 | >4.73 |
| DIFFERENTIAL | 1.353 | | | | |

| | | ANTIVIRUAL | | CYTOTOXICITY | | |
|---|---|---|---|---|---|---|
| DRUG DDC | | TEST VALUES | | TEST VALUES | | |
| ROW ON | CONC. | MEAN | % RED. IN | MEAN | % CELL | COLORIMETRIC |
| PLATE | (μM) | O.D. | VIRAL CPE | O.D. | VIABILITY | CONTROL |

| | | | | | | | | |
|---|---|---|---|---|---|---|---|---|
| BASED ON | low | B | 0.00003 | 0.014 | 1% | 1.496 | 100% | 0.010 |
| VALUES | | C | 0.0001 | 0.062 | 5% | 1.346 | 93% | −.006 |
| OF | | D | 0.00032 | 0.080 | 6% | 1.428 | 99% | −.004 |
| COLUMNS | | E | 0.001 | 0.057 | 4% | 1.476 | 100% | 0.000 |
| 7 through 12 | | F | 0.0032 | 0.109 | 8% | 1.450 | 100% | −.003 |
| (RIGHT | | G | 0.01 | 0.119 | 9% | 1.560 | 100% | −.005 |
| SIDE OF | | B | 0.032 | 1.019 | 75% | 1.658 | 100% | −.003 |
| PLATE) | | C | 0.1 | 1.099 | 81% | 1.391 | 96% | 0.002 |
| BASED ON | | D | 0.32 | 1.246 | 92% | 1.400 | 97% | 0.030 |
| VALUES | | E | 1 | 1.166 | 86% | 1.469 | 100% | 0.003 |
| OF | | F | 3.2 | 1.754 | 100% | 1.826 | 100% | −.002 |
| COLUMNS | high | G | 10 | 1.687 | 100% | 2.064 | 100% | 0.003 |
| 1 through 6 | | | | | | | | |
| (LEFT SIDE | | | | | | | | |
| OF PLATE) | | | | | | | | |

TABLE 2

| | PLATE HAX | | | IN VITRO ANTIVIRAL RESULTS | | | | | DRUG: BVS10A | | | |
|---|---|---|---|---|---|---|---|---|---|---|---|---|
| | DRUG BVS10A | | | XIT ASSAY | | | | | SI: >6.01 | | | |
| | 1 | 2 | 3 | 4 | 5 | 6 | 7 | 8 | 9 | 10 | 11 | 12 |
| | reagent background | | | | | | plastic background | | | | | |
| A | 0.179 | 0.171 | 0.165 | 0.172 | 0.177 | 0.174 | 0.095 | 0.089 | 0.091 | 0.089 | 0.095 | 0.088 |
| | lox | cc/vc | experimental = high | | | lox | lox | experimental = low | | | cc/vc | lox |
| | | | concentration | | | | | concentrations | | | | |
| B | 1.547 | 1.414 | 0.232 | 0.379 | 0.353 | 1.557 | 1.509 | 0.269 | 0.238 | 0.325 | 1.438 | 1.462 |
| C | 1.667 | 1.394 | 0.282 | 0.334 | 0.391 | 1.695 | 1.499 | 0.251 | 0.326 | 0.271 | 1.383 | 1.472 |
| D | 1.806 | 1.347 | 0.233 | 0.282 | 0.267 | 1.633 | 1.450 | 0.252 | 0.258 | 0.229 | 1.332 | 1.498 |
| E | 1.826 | 0.287 | 0.335 | 0.307 | 0.426 | 2.108 | 1.504 | 0.281 | 0.296 | 0.263 | 0.251 | 1.560 |
| F | 2.634 | 0.226 | 1.520 | 1.786 | 1.936 | 2.470 | 1.522 | 0.314 | 0.330 | 0.322 | 0.248 | 1.493 |
| G | 1.877 | 0.232 | 1.868 | 2.814 | 2.038 | 2.674 | 1.543 | 0.250 | 0.259 | 0.276 | 0.236 | 1.533 |
| | colorimetric background = | | | | | | colorimetric background = | | | | | |
| | high concentrations | | | | | | low concentrations | | | | | |
| H | 0.179 | 0.162 | 0.163 | 0.165 | 0.169 | 0.167 | 0.170 | 0.169 | 0.168 | 0.167 | 0.163 | 0.169 |

lox = cell loxicity
cc = cell control
vc = virus control
BOLD = highest drug conc
values shown are optional densities

| | | | | |
|---|---|---|---|---|
| VIRUS | HIV1 | PASSAGE - | PROJECT # | — |
| CELLS | CEMSS | PASSAGE 15 | SPONSOR | SHETTY |
| | | OPERATOR 12S | TEST DATE | Sep. 10, 1997 |
| | | | DATE READ | Sep. 16, 1997 |

| STRN | RP | DRUG BVS 10A | 25% | 50% | 95% |
|---|---|---|---|---|---|
| REAGENT | 0.173 | TC (UG/mL) | >100.00 | >100.00 | >100.00 |
| VIRUS CONTROL | 0.070 | IC (UG/mL) | 12.00 | 16.60 | 30.00 |
| CELL CONTROL | 1.212 | ANTIVIRAL INDEX (AI) | >8.33 | >6.01 | >3.34 |
| DIFFERENTIAL | 1.141 | | | | |

| | | | | ANTIVIRAL | | CYTOTOXICITY | | |
|---|---|---|---|---|---|---|---|---|
| | DRUG BVS10A | | | TEST VALUES | | TEST VALUES | | |
| | ROW ON | | CONC. | MEAN | % RED. IN | MEAN | % CELL | COLORIMETRIC |
| | PLATE | | (nG/mL) | O.D. | VIRAL CPC | O.D. | VIABILITY | CONTROL |
| BASED ON | low | B | 0.00032 | 0.038 | 3% | 1.317 | 100% | −.004 |
| VALUE OF | | C | 0.001 | 0.049 | 4% | 1.323 | 100% | −.010 |
| COLUMNS | | D | 0.0032 | 0.009 | 1% | 1.307 | 100% | −.006 |
| 7 through 12 | | E | 0.01 | 0.042 | 4% | 1.364 | 100% | −.005 |
| (RIGHT SIDE | | F | 0.032 | 0.083 | 7% | 1.339 | 100% | −.004 |
| OF PLATE) | | G | 0.1 | 0.021 | 2% | 1.369 | 100% | −.003 |
| BASED ON | | B | 0.32 | 0.051 | 4% | 1.385 | 100% | −.006 |
| VALUES OF | | C | 1 | 0.103 | 9% | 1.512 | 100% | −.004 |
| COLUMNS | | D | 3.2 | 0.025 | 2% | 1.556 | 100% | −.008 |
| 1 through 6 | | E | 10 | 0.123 | 11% | 1.804 | 100% | −.010 |
| (LEFT SIDE | | F | 32 | 1.515 | 100% | 2.390 | 100% | −.011 |
| OF PLATE) | high | G | 100 | 1.717 | 100% | 1.797 | 100% | 0.006 |

TABLE 3
AZT VS. ROJO IN PBMC

| CONC (μM) | | | | | | | | | |
|---|---|---|---|---|---|---|---|---|---|
| 4 | 1.3 | 0.4 | 0.13 | 0.04 | 0.013 | 0.004 | 0.0013 | 0.004 | 0 |

RT ACTIVITY (cpm)

| | | | | | | | | | | |
|---|---|---|---|---|---|---|---|---|---|---|
| SAMPLE 1 | 48.0 | 68.0 | 100.0 | 2679.0 | 5362.0 | 4391.0 | 4974.0 | 7727.0 | 5701.0 | 7197.8 |
| SAMPLE 2 | 52.0 | 48.0 | 602.0 | 2866.0 | 5778.0 | 7692.0 | 5443.0 | 7459.0 | 6457.0 | 7197.8 |
| SAMPLE 3 | 48.0 | 88.0 | 120.0 | 1448.0 | 5512.0 | 6218.0 | 6540.0 | 7408.0 | 5440.0 | 7197.8 |
| MEAN | 48.3 | 88.0 | 274.0 | 2397.0 | 5880.0 | 5566.3 | 5666.7 | 7531.7 | 5888.0 | 7197.8 |
| % VC | 0.7 | 8.8 | 3.8 | 32.3 | 81.7 | 77.3 | 77.6 | 104.6 | 81.5 | 100.0 |

TOXICITY VALUES (XTT - O.D. @ 450/650 nm)

| | | | | | | | | | | |
|---|---|---|---|---|---|---|---|---|---|---|
| SAMPLE 1 | 2.964 | 2.954 | 2.897 | 3.239 | 3.260 | 3.165 | 2.952 | 2.908 | 3.099 | 3.151 |
| SAMPLE 2 | 2.607 | 3.085 | 3.101 | 2.984 | 2.716 | 2.933 | 3.026 | 2.704 | 2.682 | 3.151 |
| SAMPLE 3 | 2.588 | 2.945 | 3.224 | 3.158 | 2.740 | 3.291 | 2.772 | 2.908 | 3.093 | 3.151 |
| MEAN | 2.718 | 2.968 | 3.107 | 3.120 | 2.902 | 3.130 | 2.917 | 2.840 | 2.958 | 3.151 |
| % CC | 86.2 | 84.8 | 98.8 | 89.0 | 92.1 | 99.3 | 92.6 | 90.1 | 93.9 | 100.0 |

IC50 (μM) = 0.10
TC50 (μM) = >4
TI = >40.0

TABLE 4
BVS 10A VS. ROJO IN PBMC

| CONC (μg/ml) | | | | | | | | | |
|---|---|---|---|---|---|---|---|---|---|
| 100 | 32 | 10 | 3.2 | 1 | 0.32 | 0.1 | 0.032 | 0.01 | 0 |

RT ACTIVITY (cpm)

| | | | | | | | | | | |
|---|---|---|---|---|---|---|---|---|---|---|
| SAMPLE 1 | 48.0 | 68.0 | 100.0 | 2679.0 | 6352.0 | 4391.0 | 4974.0 | 7727.0 | 6701.0 | 7197.8 |
| SAMPLE 2 | 52.0 | 48.0 | 602.0 | 2856.0 | 6776.0 | 7092.0 | 6443.0 | 7459.0 | 6457.0 | 7197.8 |
| SAMPLE 3 | 48.0 | 88.0 | 120.0 | 1446.0 | 6512.0 | 6216.0 | 6340.0 | 7409.0 | 6440.0 | 7197.8 |
| MEAN | 49.3 | 68.0 | 274.0 | 2327.0 | 6546.7 | 5899.7 | 5919.0 | 7531.7 | 6532.7 | 7197.8 |
| % VC | 0.7 | 0.9 | 3.8 | 32.3 | 91.0 | 82.0 | 82.2 | 104.6 | 90.8 | 100.0 |

TOXICITY VALUES (XTT - O.D. @ 450/650 nm)

| | | | | | | | | | | |
|---|---|---|---|---|---|---|---|---|---|---|
| SAMPLE 1 | 1.874 | 3.468 | 3.855 | 3.710 | 3.465 | 3.641 | 3.351 | 3.631 | 3.699 | 3.308 |
| SAMPLE 2 | 3.838 | 3.470 | 3.856 | 3.383 | 3.378 | 3.009 | 3.587 | 3.795 | 3.504 | 3.308 |
| SAMPLE 3 | 1.573 | 2.721 | 3.238 | 3.178 | 2.699 | 3.368 | 2.703 | 2.853 | 3.042 | 3.308 |
| MEAN | 1.695 | 3.220 | 3.583 | 3.424 | 3.181 | 3.339 | 3.214 | 3.426 | 3.415 | 3.308 |
| % CC | 51.2 | 97.3 | 108.3 | 103.5 | 96.2 | 100.9 | 97.1 | 103.6 | 103.2 | 100.0 |

IC50 (μg/ml) = 2.54
TC50 (μg/ml) = >100
TI = >39.4

### Explanation of the Results:

The two assays measure the ability of a test compound to inhibit HIV replication. In PBMCs, virus replication occurs without any evidence of cytopathic effect or cell killing. In CEM-SS cells, virus replication results in significant cytopathic effect and cell killing.

Thus, PBMCs assay measures the amount of virus released from the infected cells into the tissue culture supernatant. The activity of the test compound is measured by its ability to reduce the level of virus production relative to the virus controls (cells+virus with no added compound). In PBMC culture reverse transcriptase activity was used in the supernatant to to quantify the amount of virus production from the infected cells. Endpoint quantification is performed at 6 days post-infection. Toxicity of the compound to the target cells is measured by the incorporation of tritiated thymidine or by use of the tetrazolium dye XTT.

CEM-SS cells assay measures the ability of the compound to inhibit HIV induced cell killing. The cells are stained at 6 days post-infection with the tetrazolium dye XTT which is converted to a colored formazan product by the mitochondria of viable cells. Protection from cell killing afforded by the test compound is measured by comparison to both cell controls and virus controls. Cell killing in this system is directly related to the amount of virus produced by the infected cells and XTT-formazan production is proportional to the number of viable cells in each well. Toxicity of the compound to the target cells is measured in parallel.

### Interpretation of the Results:

The results in both assay systems indicate that BVS-10A was able to inhibit the replication of HIV-1. In both assays a high test compound concentration of 100 μg/mL was used. In PBMCs, 50% inhibition of virus replication was achieved at 2.5 μg/mL. In CEM-SS cells, 50% inhibition of cell killing was achieved at 12.0 μg/mL. Toxicity was observed in the PBMC cultures with 50% inhibition of cell growth at approximately 100 μg/mL. No toxicity at the high test concentration was observed in CEM-SS cells. AZT and ddC were used as positive anti-HIV controls in order to be sure the assay system worked as expected. The results obtained with these control compounds indicate that each of these antiviral assays of BVS-10A represent good antiviral evaluations.

### Significance of the Results:

These assays suggest that BVS-10A is an effective inhibitor of HIV-1 replication in both established and fresh human cell systems. BVS-10A exhibits a therapeutic index of approximately 50 in PBMCs and >10 in CEM-SS cells.

Detailed descriptions of both the CEM-SS cell method and the PBMC method are given below.

### CEM-SS Cells Assay

### Microtiter Antiviral XTT Assay

### Cell Preparation:

CEM-SS cells (or other established human cell line used in these experiments) were passaged in T-150 flasks for use in the assay. On the day preceding the assay, the cells were split 1:2 to assure they would be in an exponential growth phase at time of infection. On the day of assay the cells were washed twice with tissue culture medium and resuspended in fresh tissue culture medium. Total cell and viability counting was performed using hemacytometer and trypan blue dye exclusion. Cell viability was greater than 95% for the cells to be utilized in the assay. The cells were pelleted and resuspended at 2.5×10⁴ cells per ml in tissue culture medium. Cells were added to the drug-containing plates in a volume of 50 μl.

### Virus Preparation:

A pretitered aliquot of virus was removed from the freezer (−80° C.) and allowed to thaw slowly to room temperature in a biological safety cabinet. The virus was resuspended and diluted into tissue culture medium such that the amount of virus added to each well in a volume of 50 μl will be the amount determined to give complete cell killing at 6 days post-infection. In general the virus pools produced with the IIIB isolate of HIV required the addition of 5 μl of virus per well. Pools of RF virus were five to ten fold more potent, requiring 0.5-1 μl per well. TCID₅₀ calculation by endpoint titration in CEM-SS cells indicated that the multiplicity of infection of these assays ranged from 0.005-2.5.

### Plate Format:

The format of the test plate has been standardized by Southern Research Institute. Each plate contained cell control wells (cells only), virus control wells (cells plus virus), drug toxicity control wells (cells plus drug only), drug calorimetric control wells (drug only) as well as experimental wells (drug plus cells plus virus).

### XTT Staining of Screening Plates:

After 6 days of incubation at 37° C. in a 5% CO₂ incubator the test plates were analyzed by staining with the tetrazolium dye XTT. XTT-tetrazolium is metabolized by the mitochondrial enzymes of metabolically active cells to a soluble formazan product, allowing the rapid quantitative analysis of the inhibition of HIV-induced cell killing by anti-HIV test substances. On day 6 post-infection plates were removed from the incubator and observed. The use of round bottom microtiter plates allows rapid macroscopic analysis of the activity of a given test compound by the evaluation of pellet size. The results of the macroscopic observations were confirmed and enhanced by further microscopic analysis.

XTT solution was prepared daily as a stock of 1 mg/ml in PBS. Phenazine methosulfate (PMS) solution was prepared at 15 mg/ml in PBS and stored in the dark at −20° C.

### CEM-SS Cells Assay

XTT-PMS stock was prepared immediately before use by diluting the PMS 1:100 into PBS and adding 40 ul per ml of XTT solution. Fifty microliters of XTT/PMS was added to each well of the plate and the plate was reincubated for 4 hours at 37° C. Adhesive plate sealers were used in place of the lids, the sealed plate was inverted several times to mix the soluble formazan product and the plate was read spectrophotometrically at 450 nm with a Molecular Devices Vmax plate reader. Using an in-house computer program % CPE Reduction, % Cell Viability, IC_{25, 50 & 95}, TC_{25, 50 & 95} and other indices were calculated and the graphic results summary was displayed.

### PBMC Assay

### Anti-HIV Activity in Fresh Human Cells: Assay in Fresh Human T-Lymphocytes

Fresh human peripheral blood lymphocytes (PBL) are isolated from voluntary Red Cross donors, seronegative for HIV and HBV. Leukophoresed blood is diluted 1:1 with Dulbecco's phosphate buffered saline (PBS), layered over 14 mL of Ficoll-Hypaque density gradient in a 50 mL centrifuge tube. Tubes are then centrifuged for 30 minutes at 600×g. Banded PBLs are gently aspirated from the resulting interface and subsequently washed 2× with PBS by low speed centrifugation. After final wash, cells are enumerated by trypan blue exclusion and resuspended at 1×10E7/mL in RPMI 1640 with 15% Fetal Bovine Serum (FBS), 2 mM L-glutamine, 4 μg/mL PHA-P and allowed to incubate for 48-72 hours at 37° C. After incubation, PBLs are centrifuged and reset in RPMI 1640 with 15% FBS, 2 mM L-glutamine, 100 U/mL penicillin, 100 ug/mL streptomycin, 10 ug/mL gentamycin, and 20 U/mL recombinant human IL-2. PBLs are maintained in this medium at a concentration of 1-2×10E6/mL with bi-weekly medium changes, until use in assay protocol. For the PBL assay, PHA-P stimulated cells from at least two normal donors are pooled, set in fresh medium at 2×10E6/mL and plated in the interior wells of a 96 well round bottom microplate at 50 μL/well. Test drug dilutions are prepared at a 2× concentration in microtiter tubes and 100 μL of each concentration is placed in appropriate wells in a standard format. 50 μL of a predetermined dilution of virus stock is placed in each test well. Wells with cells and virus alone are used for virus control. Separate plates are identically set without virus for drug cytotoxicity studies using an XTT assay system.

In the standard PBL assay (MOI: 0.2), the assay was ended on day 7 following collection of cell free supernatant samples for reverse transcriptase activity assay. In the low MOI PBL assay (MOI: 0.02), supernatant samples were collected on day 6, day 11, and day 14 post-infection and analyzed for RT activity. Tritiated thymidine triphosphate (NEN) (TTP) was resuspended in distilled H₂O at 5 Ci/ml. Poly rA and oligo dT were prepared as a stock solution which was kept at −20° C. The RT reaction buffer was prepared fresh on a daily basis and consists of 125 μL 1M EGTA, 125 μL dH₂O, 110 μL 10% SDS, 50 μL 1M Tris (pH 7.4), 50 μL 1M DTT, and 40 μL 1M MgCl₂. These three solutions were mixed together in a ratio of 2 parts TTP, 1 part poly rA:oligo dT, and 1 part reaction buffer. Ten microliters of this reaction mixture was placed in a round bottom microtiter plate and 15 μL of virus containing supernatant was added and mixed. The plate was incubated at 37° C. in a water bath with a solid support to prevent submersion of the plate and incubated for 60 minutes. Following reaction, the reaction volume was spotted onto pieces of DE81 paper, washed 5 times for 5 minutes each in a 5% sodium phosphate buffer, 2 times for 1 minute each in distilled water, 2 times for 1 minute each in 70% ethanol, and then dried. Opti-Fluor O was added to each sample and incorporated radioactivity was quantitated utilizing a Wallac 1450 Microbetaplus liquid scintillation counter.

Tritiated thymidine incorporation was measured in parallel cultures at day 7. Each well was pulsed with 1 μCi of tritiated thymidine and the cells were harvested 18 hours later with a Skatron cell harvester onto glass fiber filter papers. The filters were dried, placed in a scintillation vial with 1 ml of scintillation cocktail and incorporated radioactivity was quantitated on a Packard Tri-Carb 1900 TR liquid scintillation counter.

### Antibacterial Test Data of “BVS-10A”

Antibacterial test data and assay method used to evaluate “BVS-10” compound.

### Test Results:

The compound BVS-10A was screened against several bacteria and the yeast, Candida albicans. The compound showed broad antibacterial activity, inhibiting the growth of all of the bacterial strains. The compound was the most active overall with minimum inhibitory concentrations (MIC) of in the range of <0.128 to 12.8 μg/mL except for C. albicans. Trimethoprim was used as a positive control drug.

The organisms are listed in Table 5 and test results are listed in Table 6.

TABLE 5

| Strain | Abbreviation | Source |
|---|---|---|
| Staphylococcus | Sa | ATCC 6538 |
| aureus | | |
| Staphylococcus | Se | ATCC 35984 |
| epidermidis | | |
| Streptococcus | Sp | ATCC 6303 |
| pneumoniae | | |
| Escherichia | Ec | ATCC 11229 |
| coli | | |
| Klebsiella | Kp | ATCC 4352 |
| pneumoniae | | |
| Pseudomonas | Pa | ATCC 15442 |
| aeruginosa | | |
| Proteus mirabilis | Pm | ATCC 9921 |

TABLE 6

| | MIC (μg/ml) | | | | | | |
|---|---|---|---|---|---|---|---|
| Compound | Sa | Se | Sp | Ec | Kp | Pa | Pm |
| BVS10A | <0.128 | <0.128 | >1.28 ≦ 12.8 | >0.128 ≦ 1.28 | <0.128 | >1.28 ≦ 12.8 | >0.128 ≦ 12.8 |
| TMP | >0.064 ≦ 0.64 | >64 | >0.64 ≦ 6.4 | >0.064 ≦ 0.64 | >0.064 ≦ 0.64 | >64 | >0.64 ≦ 6.4 |

BVS-10A was assayed for antimicrobial activity following Clinical Laboratory Standard Institute (CLSI) standards against 30 strain diversity sets of Category A and B Bacterial pathogens Burkholderia pseudomallei, Francisella tularensis, Burkholderia mallei, Bacillus anthracis, and Yersinia pestis.

The minimum inhibitory concentration (MIC) is determined for each compound against each individual isolate. The minimum concentrations of antibiotic that result in 90% inhibition of bacterial growth (MIC90) and 50% inhibition (MIC50) are identified for each compound against the entire diversity set. These values are determined both by visual inspection and spectrophotometry. When there is a discrepancy between the visual and spectrophotometric MIC, the visual MIC is used in the report and data analysis. All compounds are tested with the recommended CLSI reference quality control (QC) strains, Escherichia coli (ATCC 25922), Staphylococcus aureus (ATCC 29213) and Pseudomonas aeruginosa (ATCC 27853) for gram (−) and (+) bacteria.

Geographically bio-diverse sets of thirty strains each of Yersinia pestis, Francisella tularensis, Bacillus anthracis, Burkholderia mallei, and Burkholderia pseudomallei were used for MIC determination in this study. Use of these “diversity” sets establishes reasonable MIC90 and MIC50 values for each compound against the holistic diversity set. A compound's performance against naturally occurring or unknown strains of each pathogen is expected to fall within the MIC90 and MIC50 values established, because the diversity set provides a good representation of isolates that may be encountered anywhere in the world.

Bacterial inoculums were prepared by suspending colonies into cation-adjusted Mueller-Hinton broth (CAMHB) from 18-24 h B. anthracis, B. pseudomallei, B. mallet plates; or 42-48 h F. tularensis and Y. pestis plates that were incubated at 35° C. Sheep Blood agar (SBA) plates were used for B. anthracis and Y. pestis, chocolate agar for F. tularensis and Trypticase Soy agar (TSA) for B. pseudomallei and B. mallei. Suspended cultures were diluted with CAMHB to a bacterial cell density of 105 CFU/ml adjusted based on OD600. Conversion factors used for each pathogen: B. anthracis: 3.82×10⁷ CFU/ml/OD; B. mallei & B. pseudomallei: 5.0×10⁸ CFU/ml/OD, Y. pestis: 5.34×10⁸ CFU/ml/OD, F. tularensis: 3.89×10¹⁰ CFU/ml/OD. 50 μl of the adjusted dilution was added to each well of the 96-well plate for a final inoculum of ˜5×10⁴ CFU/well.

MICs were determined by the microdilution method in 96-well plates according to CLSI guidelines. Antibiotics were serially diluted two-fold in 50 μl of CAMHB. For all steps with F. tularensis, CAMHB was supplemented with 2% Isovitalex (Becton Dickinson). The antibiotic ranges were 8-0.0039 μg/ml or 64-0.03125 μg/ml based on a final well volume of 100 μl after inoculation. Plates were incubated at 35° C. MICs were determined visually at 18-24 h or 42-48 h (FT and YP) and also by reading the plates at 600 nm (SpectroMax M2, Molecular Devices).

Quality control of antibiotic stocks was established by using E. coli ATCC 25922, P. aeruginosa ATCC 27853 and S. aureus ATCC 29213. Inoculums were prepared as described in sect 3.1 from 18-24 h SBA plates. Conversion factors; E. coli 6.83×10⁸ CFU/ml/OD, P. aeruginosa 5.74×10¹⁰ CFU/ml/OD and S. aureus 2.07×10¹⁰ CFU/ml/OD.

BVS-10A, as a dry powder, was made into antibiotic stocks of 5.15 mg/ml in 100% DMSO. Prior to use, this solution was further diluted in CAMHB for loading onto 96 microwell plates as described above. Comparative antibiotics ciprofloxacin, ceftazidime, and azithromycin were made into 5.15 mg/ml stocks and stored at −70° C. until use.

BVS-10A was received as a dry powder. Antibiotic stocks of 5.15 mg/ml were made in 100% DMSO. Prior to use, this solution was further diluted in CAMHB for loading onto 96 microwell plates as described in the experimental protocol section above. Comparator antibiotics ciprofloxacin, ceftazidime, and azithromycin were all purchased from USP, made into 5.15 mg/ml stocks according to the CLSI M100-S18 Table 4 guidelines and stored at −70° C. until use.

The quality control MIC results were found to be consistent with past results. To rule out the effect of DMSO on the apparent performance of the compounds where stocks were solubilised in DMSO, serial dilutions of DMSO made and MIC values for the “wild type” variant for each pathologic strain, in addition to QC strains were determined. The lowest DMSO percentage tolerated (read as MIC50) by the pathogenic bacteria was 6.25%. For the QC strains, the lowest was for P. aeruginosa at 3.125%. The greatest percentage of DMSO that existed in the evaluated compound was 1.6% (in the first well only, with serial dilutions to follow) and not considered to influence the values obtained.

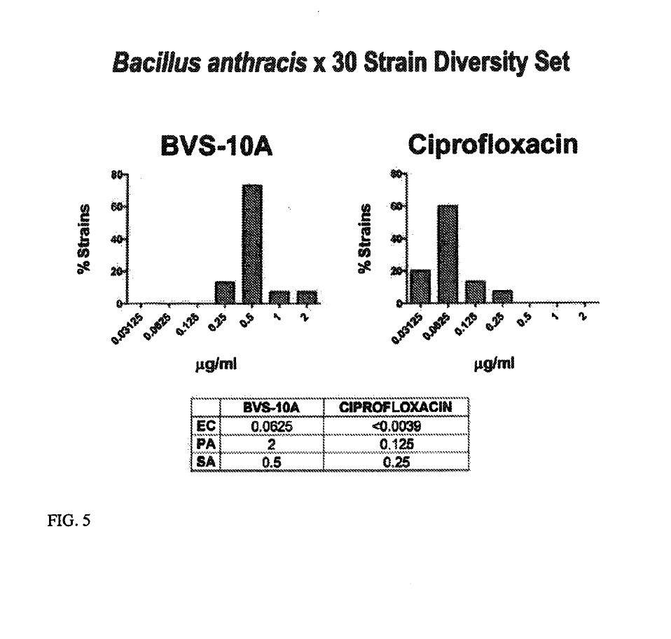

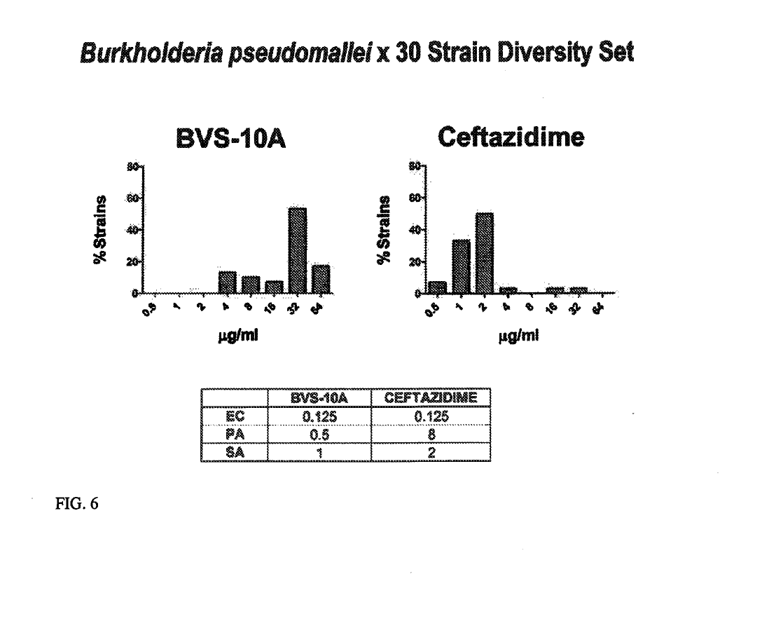

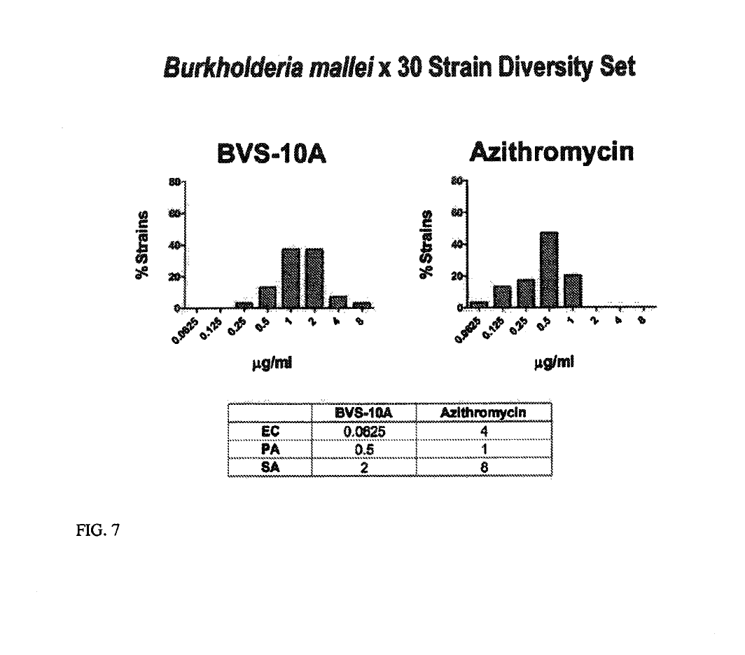

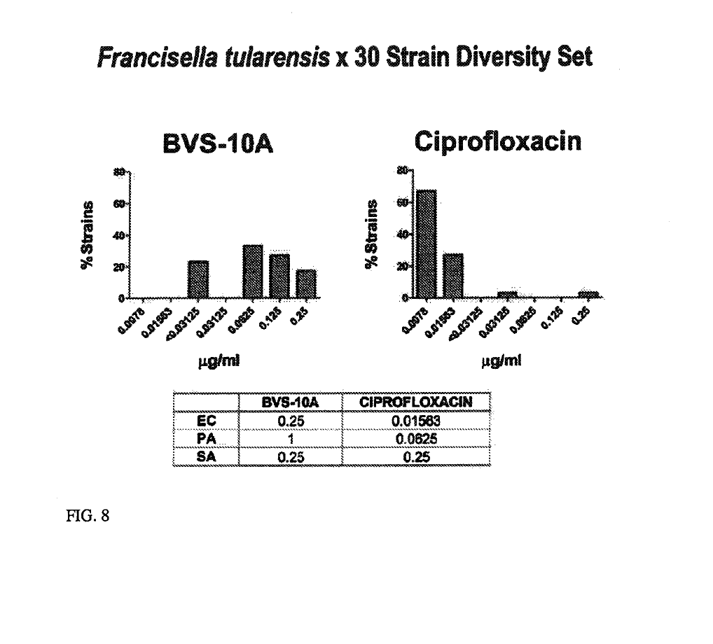

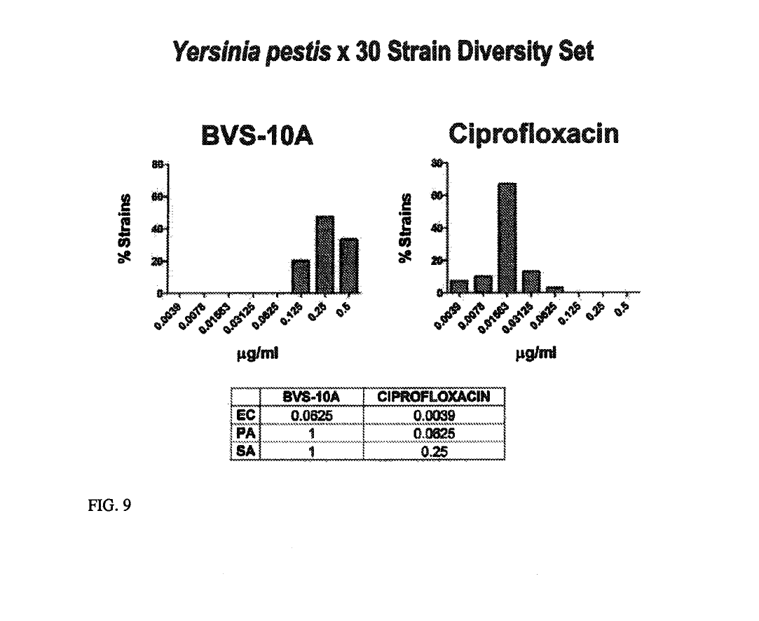

Overall, FIGS. 5-9 demonstrate that BVS-10A showed impressive results with MIC90 values ≧4 μg/ml against the B. anthracis, F. tularensis, Y. pestis and B. mallei diversity sets.

The comparator antibiotic, ciprofloxacin, had MIC90=0.125 against the B. anthracis diversity set. Though not quite as low, BVS-10A still had MIC90=1. BVS-10A was more effective against the F. tularensis diversity set. Its MIC90 value was 0.25, compared to the MIC90=0.01563 of the comparator antibiotic, ciprofloxacin.

Because ciprofloxacin is also quite effective in vitro against the Y. pestis diversity set, it did not surprise that BVS-10A could not match its low MIC90=0.03125. However, BVS-10A had an impressive MIC90=0.5.

Compounds tend to have considerably elevated MIC values against the B. mallei diversity compared to anthrax, tularaemia, and plague. The comparator antibiotic, azithromycin, had MIC90=1 and BVS-10A was close with an MIC90=4.

Against the notoriously difficult B. pseudomallei diversity set, the comparator antibiotic, ceftazidime, had MIC90=4. While BVS-10A showed some activity, its MIC90 was still just 64.

In general, a 4 μg/ml in vitro MIC value supports the progression of an antibiotic into further (in vivo) studies, because this value is reasonably achievable in serum, without regard for tissue accumulation or individual drug performance. Of course, this is highly dependent on solubility, bio-availability, and the overall physiological characteristics of the drug.

Based on the results of in vitro testing, BVS-10A should be further tested in vivo against 4 of the 5 biothreat pathogens (F. tularensis, Y. pestis, B. anthracis and B. mallei). Considering these 4 pathogens alone, BVS-10A had MIC values ≧4 μg/ml against all but one (GB5 strain, B. mallei) of 120 different isolates. Furthermore, while its MIC90 value was 64 μg/ml against the notoriously recalcitrant Burkholderia pseudomallei, BVS-10A still had MICs<16 μg/ml against 30% (9/30) of the isolates.

BVS-10A is a surprisingly effective treatment alternative and is especially valuable and unique where current therapies are not indicated due to host drug reactions or resistance. BVS-10A shows true broad spectrum activity in vitro. It is useful in methods of treating Gram negative and Gram positive infections.

### MIC Summary for BVS-10A

| Y. pestis | BVS-10A | Ciprofloxacin |
|---|---|---|
| MIC Range (μg/ml) | 0.125-0.5 | 0.0039-0.0625 |
| MIC₅₀ | 0.25 | 0.01563 |
| MIC₉₀ | 0.5 | 0.03125 |
| B. anthracis | BVS-10A | Ciprofloxacin |
| MIC Range (μg/ml) | 0.25-2 | 0.03125-0.25 |
| MIC₅₀ | 0.5 | 0.0625 |
| MIC₉₀ | 1 | 0.125 |
| F. tularensis | BVS-10A | Ciprofloxacin |
| MIC Range (μg/ml) | <0.03125-0.25 | 0.0078-0.25 |
| MIC₅₀ | 0.0625 | 0.0078 |
| MIC₉₀ | 0.25 | 0.01563 |
| B. mallei | BVS-10A | Azithromycin |
| MIC Range (μg/ml) | 0.25-8 | 0.0625-1 |
| MIC₅₀ | 1 | 0.5 |
| MIC₉₀ | 4 | 1 |
| B. pseudomallei | BVS-10A | Ceftazidime |
| MIC Range (μg/ml) | 4-64 | 0.5-32 |
| MIC₅₀ | 32 | 2 |
| MIC₉₀ | 64 | 4 |

### Anti-Tubercular Testing

BVS-10A was tested for activity against Mycobacterium tuberculosis (Mtb). This project was funded in whole or in part with Federal funds from the Division of Microbiology and Infectious Diseases, National Institute of Allergy and Infectious Diseases, National Institute of Health, Department of Health and Human Services, under Contract No. HHSN2722011000121.

Assays.

Subsequent to initial dose response testing, the test compound was subjected to secondary assays for evaluation of antimycobacterial activity in a low throughput format. These assays included MIC, MBC, LORA, intracellular (macrophage) drug screening, and MTT cell proliferation. Listed below are abbreviated summaries of each assay.

Minimal Inhibitory Concentration (MIC).

The broth microdilution assay format following guidelines established by the Clinical and Laboratory Standards Institute (CLSI) is routinely utilized for MIC testing. Briefly, testing was conducted using 96-well, U-bottom microplates with an assay volume of 0.2 mL/well. First, the test media, Middlebrook 7H9 broth supplemented with OADC Enrichment (BD BioSciences; Sparks, Md.), was added (0.1 mL/well) to each well. The test compounds, solubilized in appropriate solvent and subsequently diluted in test media, were subsequently added (0.1 mL/well) to appropriate wells at twice the intended starting concentration and serially diluted two-fold across the plate. The plates were then inoculated (0.1 mL/well) with a targeted concentration of 1.0×10⁶ CFU/mL M. tuberculosis and incubated at 37° C. for 7 days in approximately 90% humidity. Following incubation, the plates were read visually and individual wells scored for turbidity, partial clearing or complete clearing. Testing was conducted in duplicate and the following controls were included in each test plate: i) medium only (sterility control); ii) organism in medium (negative control); and iii) rifampin or isoniazid (positive control). The MIC is reported as the lowest concentration (μg/mL) of drug that visually inhibits growth of the organism.

Minimal Bactericidal Concentration (MBC).

The MBC is determined subsequent to MIC testing by sub-culturing diluted aliquots from wells that fail to exhibit macroscopic growth. The sample aliquots were inoculated onto Middlebrook 7H10 agar plates and subsequently incubated for 16-21 days at 37° C. Once growth was readily apparent, the bacterial colonies were enumerated. The MBC is defined as the lowest concentration (μg/mL) of compound exhibiting 99.9% kill over the same time period used to determine the MIC (18-24 h). MBC values greater than 16 times the MIC typically indicate antimicrobial tolerance.

Low-Oxygen Recovery Assay (LORA).

The low oxygen recovery assay (LORA) assay provides assessment of activity against Mtb in a state of nonreplicating persistence (NRP). This is important because nonreplicating Mtb is thought to be a factor in antimicrobial tolerance that occurs with some infections. The intracellular (macrophage) drug screening assay evaluates intracellular drug effectiveness. This is important because Mtb can survive inside macrophages which contributes to treatment failure and disease relapse. Traditional screening of drugs against M. tuberculosis only addresses or targets the organism in an active replicating state. It is well documented that Mtb can reside in a state of non-replicating persistence (NRP) which has not been adequately assessed in the development of new antimicrobials. Briefly, microplates were prepared in the same manner as the MIC testing format. Instead of incubating aerobically, the plates are placed under anaerobic conditions using a MACS MIC automated jar gassing system and incubated for 7 days at 37° C. The plates were subsequently transferred to an ambient gaseous condition (5% CO2) for 7 days after which the plates are read visually and individual wells scored for turbidity, partial clearing or complete clearing. Testing was conducted in duplicate and the following controls were included in each test plate: i) medium only (sterility control); ii) organism in medium (negative control); and iii) rifampin or isoniazid (positive control). Results are reported as the lowest concentration (μg/mL) of drug that visually inhibits growth of the organism.

Intracellular Drug Activity.

Briefly, the murine J774 cell line was propagated in RPMI 1640 supplemented with L-glutamine and fetal bovine serum (FBS). Cells were maintained in tissue culture flasks at 37° C. in the presence of 5% CO2. For infection studies, J774 cells were transferred to 12-well tissue culture chambers in 1 mL volumes at a density of 2.0×10⁵ in the presence of 10% FBS. After overnight incubation, the medium was replaced with fresh medium containing 1% FBS to stop macrophage division while maintaining cell viability. Twenty-four hours later, the macrophage monolayer was enumerated with an ocular micrometer for total number of cells per well to determine the infection ratio. The medium was removed and replaced with 1 mL of fresh medium with 1% FBS containing Mtb at a multiplicity of infection (MOI) of 5 Mycobacteria/macrophage. The cells are infected for 4 h after which time nonphagocytosed Mycobacteria were washed from the monolayers and fresh medium added. Drugs were then added, using 3 concentrations, and infection allowed to proceed for 7 days. At 0 and 7 days, the macrophages were lysed with sodium dodecyl sulfate, treated with DNAase, diluted and plated onto 7H10 agar to determine the cell number or colony forming units (CFU). Each drug concentration wasted in duplicate and rifampin was used as the positive control drug. A drug cytotoxicity control plate assay (MTT proliferation) was also conducted in parallel using uninfected macrophages to confirm that concentrations utilized for testing were not toxic to the macrophages.

Bacteria.

MIC screening was conducted for Mtb H37Rv (SRI 1345), isoniazid (INH)-resistant Mtb (SRI 1369), rifampin (RMP)-resistant Mtb (SRI 1367), and ofloxacin (OFX)-resistant Mtb (SRI 4000). MBC, LORA and intracellular drug screening assays were conducted using only Mtb H37Rv (SRI 1345).

MIC.

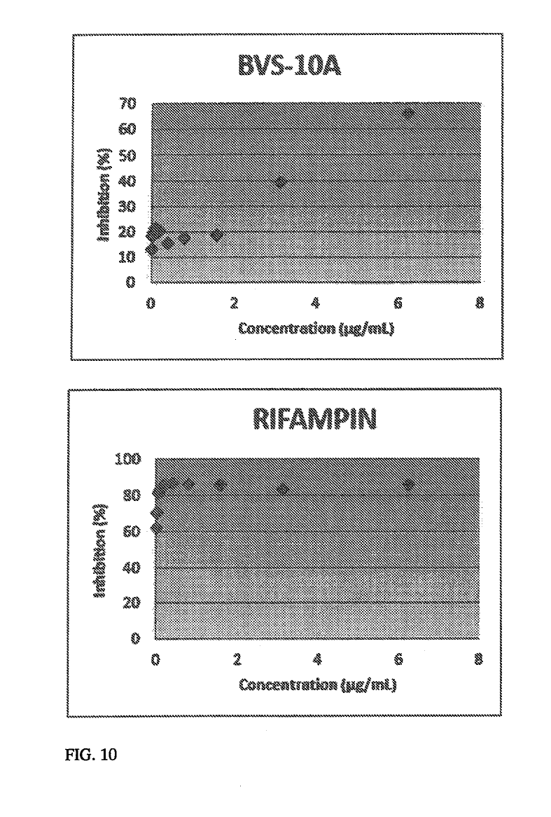

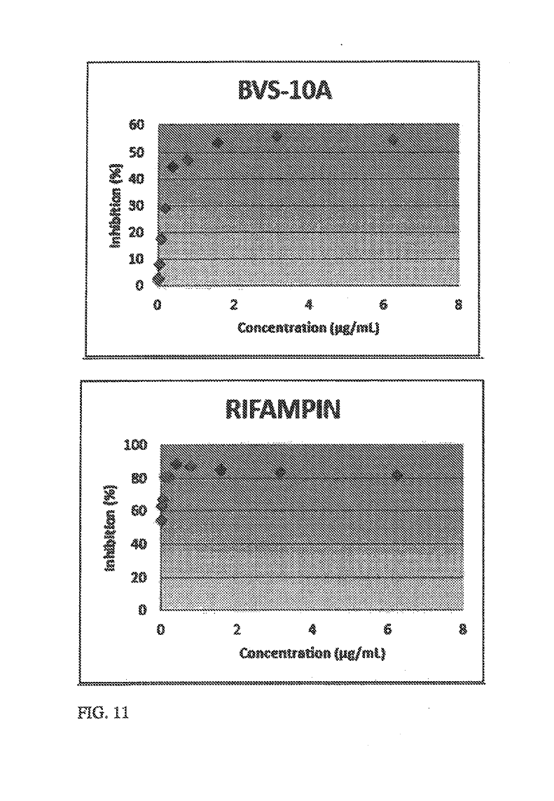

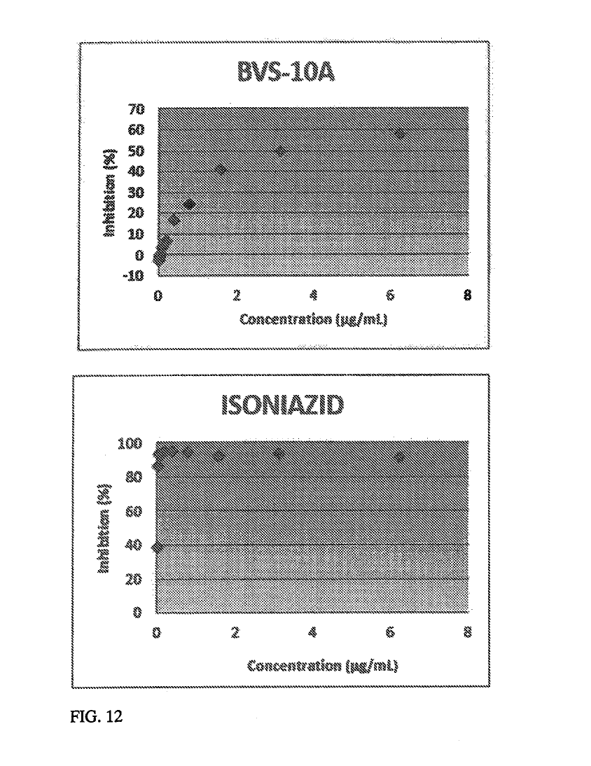

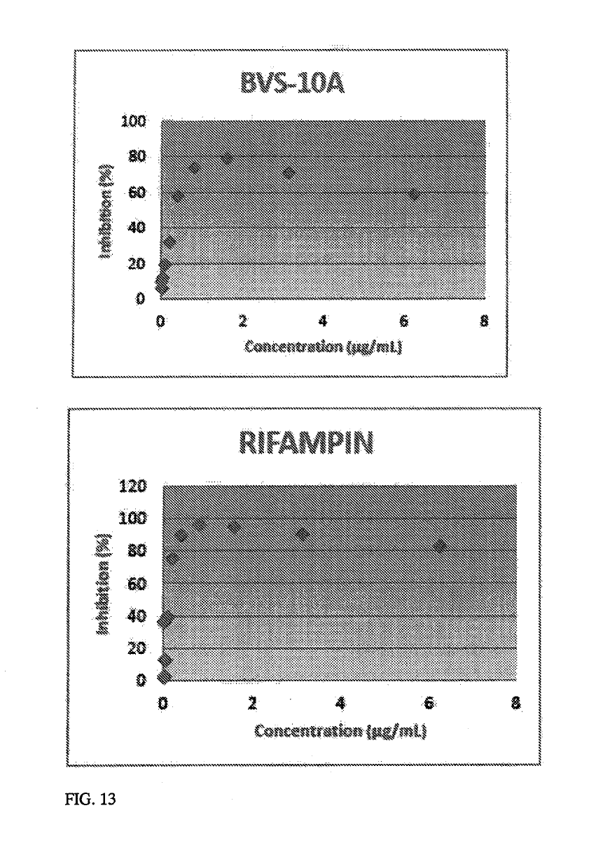

The MIC for each compound was determined by testing ten, two-fold dilutions in concentration ranges provide in Table 2. The MIC is reported as the lowest concentration (μg/mL) of drug that visually inhibited growth of the organism. In addition, the percentage of inhibition at the MIC is provided. FIG. 10 shows the dose response graphs for Mtb H37Rv. FIG. 11 shows the dose response graphs for Isoniazid Resistance. FIG. 12 shows the dose response graphs for Rifampin Resistance. FIG. 13 shows the dose response graphs for Ofloxacin Resistance.

MBC.

The established rejection value of >40 colonies for the MBC assay was based on the calculated concentration of Mtb in the MIC plates. Results, reported as μg/mL concentration, are determined based on Colony Forming Units (CFUs) enumerated from agar plates. Only agar plates with countable colonies have reportable counts. If a compound lacks bactericidal activity, many times the CFUs are too numerous to count (TNTC) and are thus reported as such.

LORA.

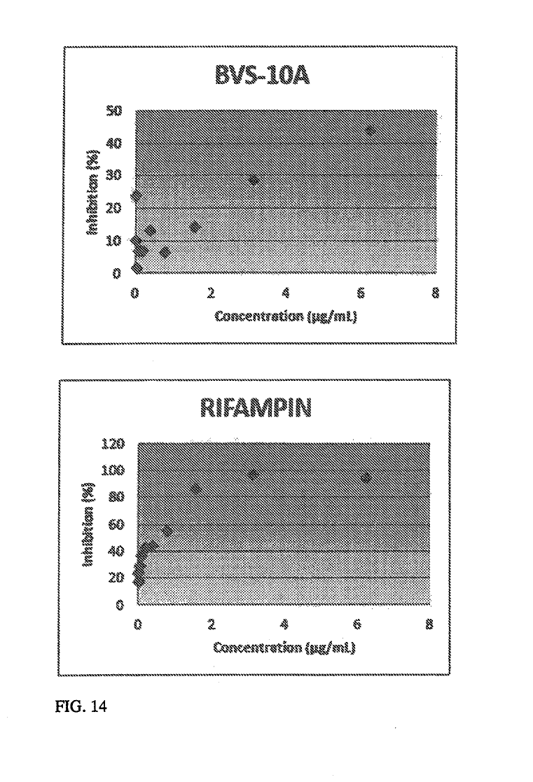

Results for the LORA assay are reported as the lowest concentration (μg/mL) of drug that visually inhibited growth of the organism in FIG. 14.

Intracellular Drug Activity.

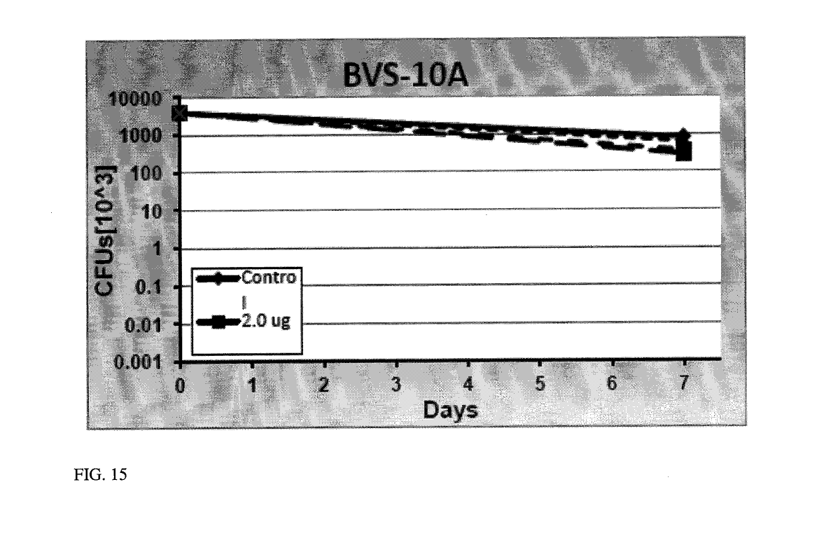

Intracellular drug activity is reported as log reduction values calculated as reduction in Mtb concentration from zero hour to 7 days post-infection. The three concentrations chosen were based on the MIC data generated in the HTS primary screen. The mid concentration bracketed the reported MIC with the lower concentration ten-fold below the mid and the higher concentration ten-fold above the mid. All data points in FIG. 15 are represented in the scatter graphs. Drug cytotoxicity is reported as cell proliferation, or percentage of viability.

### BVS-10A Concentration Ranges and Values Used for Screening

| | | LORA | Macrophage | |
|---|---|---|---|---|
| NIAID List | MIC (μg/ml) | (μg/ml) | (μg/ml) | MTT (μg/ml) |
| 50 top: | 6.25-0.012 | 6.25-0.012 | 2, 0.2. 0.02 | 2, 0.2. 0.02 |
| Apr. 23, 2012 | | | | |

### MIC and MBC Results

| | MIC | | MIC | MIC | | MIC | | MIC | |
|---|---|---|---|---|---|---|---|---|---|
| NIAID Supplier | H [symbol] | % | H [symbol] | INH-R^{b} | % | RMP-R^{c} | % | OFX-R^{d} | % |
| ID | (μg/ml) | inhibition^{a} | (μg/ml) | (μg/ml) | inhibition | (μg/ml) | inhibition | (μg/ml) | inhibition |
| BVS-10A | 6.25 | 66 | NA^{e} | 1.56 | 53 | 6.25 | 58 | 0.781 | 74 |
| Rifampin | 0.049 | 81 | 3.125 | 0.049 | 67 | NA^{f} | NA | 0.195 | 75 |
| (pos control) | | | | | | | | | |
| Isoniazid | NA | NA | NA | NA | NA | 0.02 | 86 | NA | NA |
| (pos control) | | | | | | | | | |

^{a}Percent inhibition at the MIC concentration
^{b}INH-R = Isoniazid Resistance
^{c}RMP-R = Rifampin Resistance
^{d}OFX-R = Ofloxacin Resistance
^{e}NA = Not Applicable: Colony Counts above the established rejection value of ≧40
^{f}NA = Not Applicable: Compound not used in assay [symbol]
indicates data missing or illegible when filed

| Compound ID | MBC (μg/mL) | Colony Count (CFU) |
|---|---|---|
| BVS-10A | —^{a} | TNTC |
| RIFAMPIN^{c} | 3.125 | 23 |

^{a}MBC above the established rejection value of [symbol] 40 colonies [symbol]
TNTC = Too Numerous To Count
^{c}MBC above expected value; however controls for the MIC assay were within range so testing was not repeated [symbol]
indicates data missing or illegible when filed

### LORA, Macrophage and MTT Results

| | Microphage | Microphage | Microphage | MTT | MTT | MTT |
|---|---|---|---|---|---|---|
| LORA | log reduction | log reduction | log reduction | % viability | % viability | % viability |
| (μg/ml) | (low conc.) | (mid conc.) | (high conc.) | (low conc.) | (mid conc.) | (high conc.) |
| BVS- | >6.25 | 1.14 | 0.80 | >100 | 63 | 73 |
| 10A | | | | | | |
| 1.56 | 0.76 | 1.11 | 2.00 | 97 | 96 | 86 |

### Antibacterial Assay

Test organisms. The test organisms are listed in Table 5 along with their source. Prior to assay, overnight bacterial slant cultures were used to inoculate Mueller Hinton broth followed by incubation for 5-7 hr. Each culture was then standardized turbidimetrically to a viable to count of about 10⁶ colony forming units/ml and used to inoculate the mirotiter assay plates. The viable count after addition to the plates was about 5×10⁵ CFU/ml.

Experimental design. The MIC of the test compounds for the bacterial strains was determined with a microdilution broth assay in 96 well microtiter plates. The test compounds were dissolved in DMSO and then diluted 10-fold in assay medium (Mueller Hinton broth) to obtain final concentrations of 0.128, 1.28, 12.8, and 128 μg/ml. A viability control containing DMSO, diluted in the same manner as the test compounds, was used to confirm that the DMSO was not inhibitory by itself. Trimethoprim (TMP) was used as a positive drug control. The assay plates were incubated at 37° C. for about 17 hr and each assay well observed visually for growth. The MIC was determined at the lowest concentration of drug which inhibited growth.

The following exemplary compounds were found to have dual anti-viral and antibacterial activity:

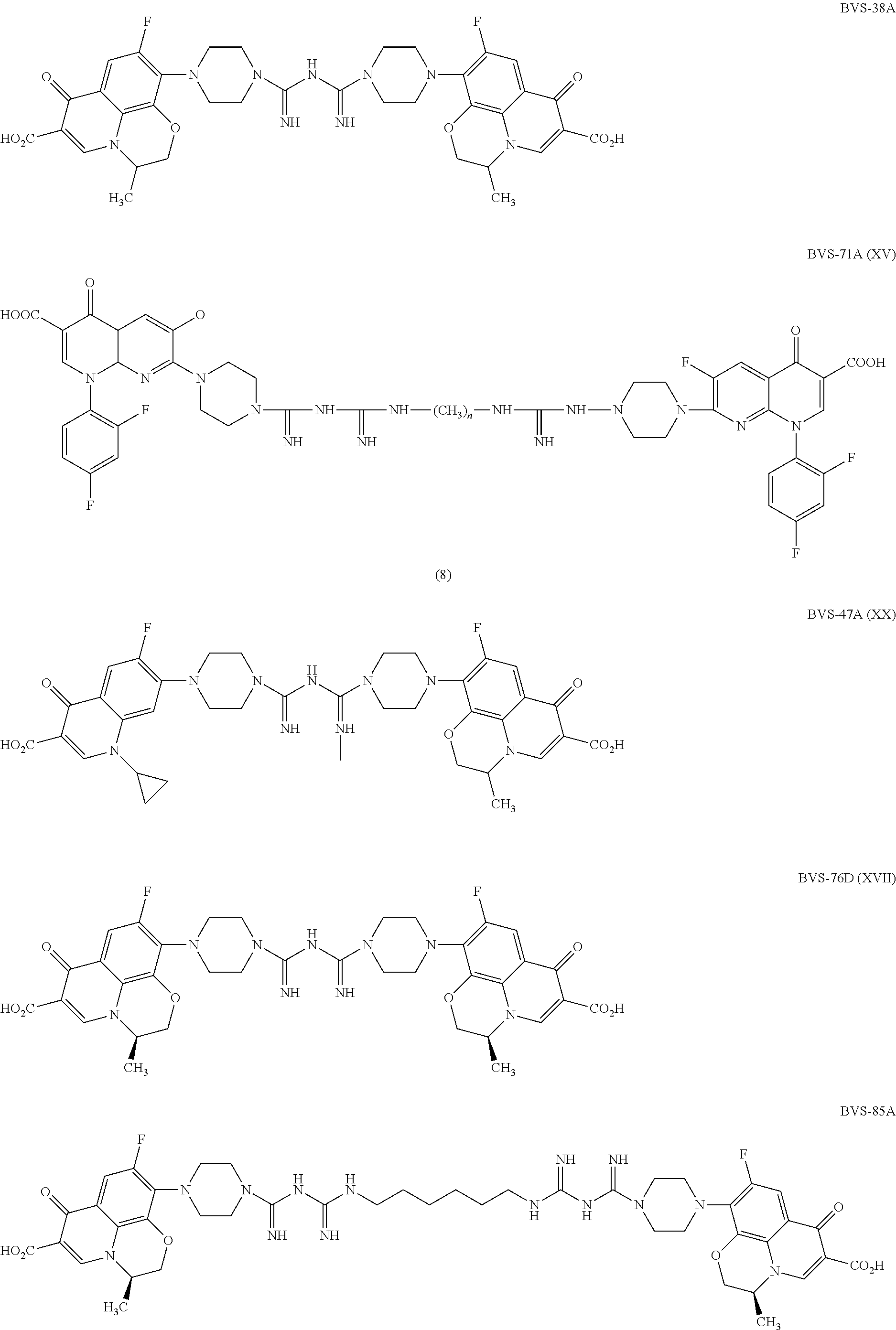

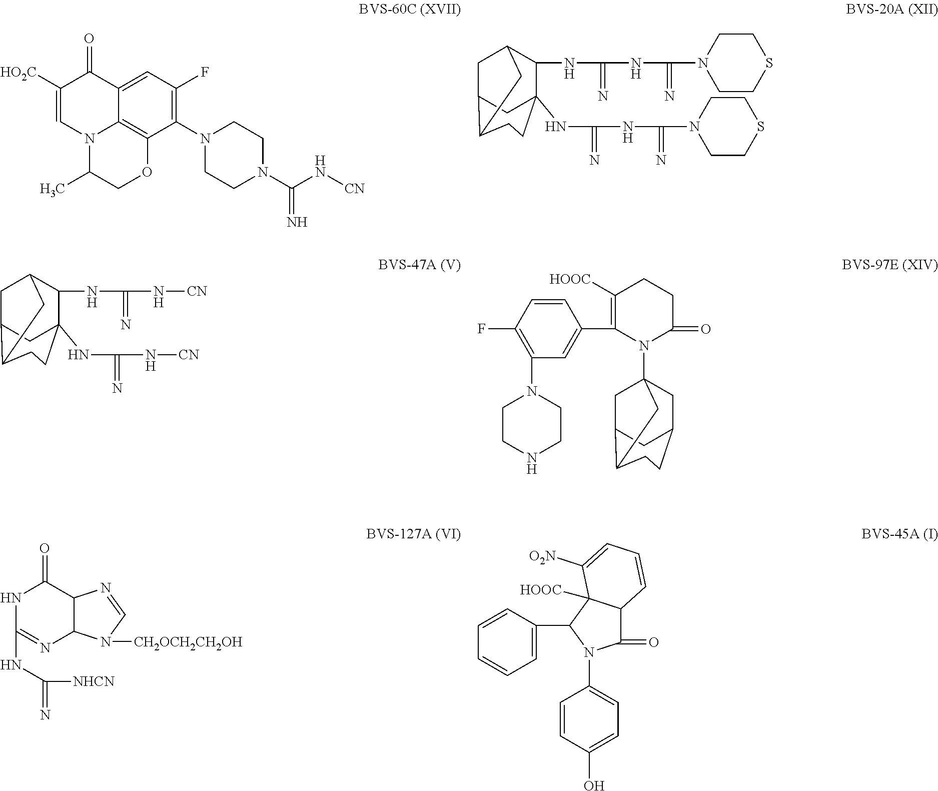

The compounds BVS-76D (XVII), BVS-85A (XVII) and BVS-60C(XVII) are each optically, stereochemically active. BVS-38A is a racemic mixture.

The compounds disclosed herein and derivatives thereof have the advantage of potent anti-microbial and anti-viral activity, e.g., against category A and B bacterial pathogens including Yersinia pestis, Bacillus anthracis, Francisella tularensis, Burkholderia pseudomallei, and Burkholderia mallei, Escherichia coli, Pseudomonas aeruginosa, Mycobacterium tuberculosis, as well as influenza A H3N2, influenza A H1N1, and HIV. In certain embodiments, the compounds and derivatives thereof also have potent anti-malarial (Plasmodium falciparum) activity. In certain embodiments, the compounds and derivatives thereof can be used to treat an ocular disease. Ocular diseases may include bacterial conjunctivitis, viral conjunctivitis, allergies, glaucoma, cataract, corneal disease, vitreo-retinal diseases, diseases and disorders of the optic nerve, oculosystemic diseases and disorders, diseases and disorders of the uvea and/or a diabetic eye disease. In one embodiment, the corneal diseases can be selected from but not limited to corneal abrasion, conjunctivitis (pink eye), corneal infections, Fuchs' Dystrophy, Herpes Zoster (shingles), Iridocorneal Endothelial Syndrome, keratoconus, Lattice Dystrophy, Map-Dot-Fingerprint Dystrophy, ocular Herpes, pterygium, Stevens-Johnson Syndrome, diabetic retinopathy, cataract and/or glaucoma, diabetic retinopathy, macular degeneration, retinal detachments or tears, macular holes, retinopathy of prematurity, retinoblastoma, uveitis, eye cancer, flashes and floaters, retinitis pigmentosa, ocular edema, adenoma, uveitis, scleritis, neuritis, and/or papilitis.

BVS-127A(VI) was found to be particularly effective for treating ocular diseases.

In certain embodiments, the inventive compounds and derivatives thereof can be used to treat, prevent or inhibit opportunistic infections.

### Anti-Plague Test Data

The protective efficacy of a range of BVS-76D (XVII) dosages against pneumonic plague (Y. pestis) in BALB/c mice was tested. Saline was used as the diluent for BVS-76D (XVII). Sixty naive female BALB/c mice were purchased from Jackson Laboratories for this study. The mice were 5-6 weeks old and the average weight was 21.6 grams. The animals were in good health, and free of malformations and clinical signs of disease. No animal developed an illness or injury unrelated to this study prior to the initial challenge or after treatment. Therefore, no replacement mice were added into the study. Upon arrival to the facility, mice acclimated for several days prior to manipulation. During this time the animals were randomized into the following treatment groups.

TABLE 7

| | | | | | | Challenge |
|---|---|---|---|---|---|---|
| | | | | | | Administration |
| | Group | Test Item Administration | | | | Y. pestis Target |
| Group | Size | Description | Dose | Route | Schedule | Concentration |
| 1 | 9 | Vehicle | 0.05% | i.p. | Once for 6 days | 5-10 LD₅₀ |
| | | control | | | beginning 24 h | |
| | | (DMSO) | | | post-challenge | |
| 2 | 9 | Levofloxacin | 5 mg/kg/day | i.p. | Once for 6 days | 5-10 LD₅₀ |
| | | | | | beginning 24 h | |
| | | | | | post-challenge | |
| 3 | 9 | BVS-76D | 5 mg/kg/day | i.p. | Once for 6 days | 5-10 LD₅₀ |
| | | (XVII) | | | beginning 24 h | |
| | | | | | post-challenge | |
| 4 | 9 | BVS-76D | 10 mg/kg/ | i.p. | Once for 6 days | 5-10 LD₅₀ |
| | | (XVII) | day | | beginning 24 h | |
| | | | | | post-challenge | |
| 5 | 9 | BVS-76D | 25 mg/kg/ | i.p. | Once for 6 days | 5-10 LD₅₀ |
| | | (XVII) | day | | beginning 24 h | |
| | | | | | post-challenge | |
| 6 | 9 | BVS-76D | 50 mg/kg/ | i.p. | Once for 6 days | 5-10 LD₅₀ |
| | | (XVII) | day | | beginning 24 h | |
| | | | | | post-challenge | |
| 7 | 6 | Toxicity | 50 mg/kg/ | i.p. | Once for 6 days | — |
| | | Control | day | | simultaneous | |
| | | Group BVS- | | | with other groups | |
| | | 76D (XVII) | | | | |

Y. pestis C092 strain was administered intranasally to all animals in Groups 1-6 on Day 0. Back titration of the challenge inoculum revealed that animals were infected with 10 LD₅₀ Y. pestis organisms. Twenty-four hours after infection, animals in Group 2 were treated with 5 mg/kg/day Levofloxacin once daily via the i.p. route for 6 consecutive days. Animals in Groups 3-6 were treated with varying doses of BVS-76D (XVII) once daily for 6 consecutive days via the i.p. route, also beginning 24 hours post-infection. On each day of BVS-76D (XVII) administration, the test compound was first dissolved in minimal amounts of DMSO, then further diluted to the required dose in saline before injection into the mice. Group 7 was the toxicity control group, and, therefore animals were treated with 50 mg/kg/day BVS-76D (XVII) for 6 days, but were not challenged with Y. pestis. All animals on the study were evaluated for 21 days post-challenge and survival was recorded on a log sheet.

Animals belonging to the toxicity control group, Group 7, were treated with the highest dose of BVS-76D (XVII) (50 mg/kg/day) but were not challenged with Y. pestis. The purpose of this group was to evaluate potential toxicity that could result from the BVS-76D (XVII) therapy itself, not in the context of a plague infection. These animals were visually observed daily for 21 days following treatment.

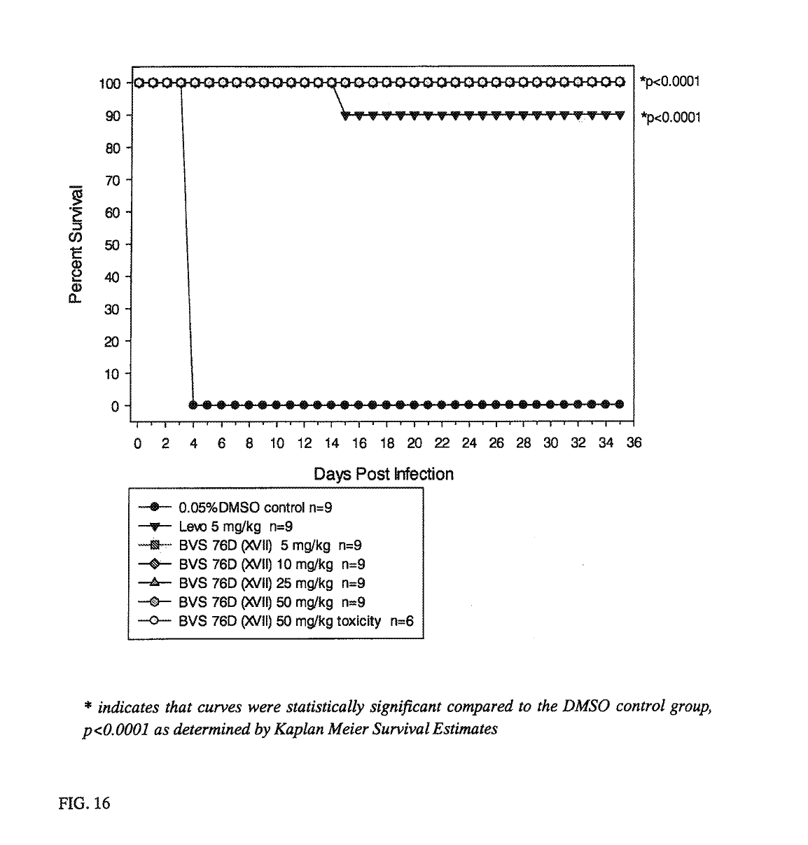

As the survival graph in FIG. 16 shows, BVS-76D (XVII) provided complete protection to mice infected with 10 LDSO Y. pestis, even at the lowest tested dose of 5 mg/kg/day. All of the vehicle control animals that received 0.05% DMSO (Group 1) succumbed to infection by Day 4 post-challenge, thus representing a proper negative control group. Levofloxacin (5 mg/kg/day) treatment resulted in 88.9% survival. However, all of the BVS-76D (XVII) doses, ranging from 5-50 mg/kg/day, afforded 100% survival to the animals. All treatment curves were statistically significant compared to the 0.05% DMSO control group, Group 1 (p<0.0001 as determined by Kaplan-Meier survival estimates).

Symptoms of all animals (Groups 1-7) were also documented throughout the study. Animals belonging to the Levofloxacin group, Group 2, never showed any outward indication of plague infection. Animals in the DMSO control group (Group 1) as well as animals that received BVS-76D (XVII) treatment (Groups 2-7) showed some inflammation in the urinary, vaginal and rectal areas on Day 2 post-infection. By Day 3 post-infection, the DMSO control group (Group 1) appeared moribund and, as expected, showed typical signs of plague infection (i.e., hunched backs, lethargy, ruffled fur). The BVS-76D (XVII) treated groups (Groups 2-7) still showed signs of inflammation on this day. On Day 4 post-infection, it was noted that all DMSO control animals were dead by this time point, and the inflammation observed in Groups 2-7 was subsiding. By Day 5 post-infection, the inflammation in Groups 2-7 was completely gone and did not reappear throughout the rest of the study. Because similar inflammatory symptoms were noted in animals that were given only the test compound (Group 7), it appeared that this inflammatory reaction was associated with DMSO.

Overall, these data indicate that BVS-76D (XVII) is quite effective against virulent Y. pestis in the pneumonic plague intranasal mouse model. Although not statistically significant, the 5 mg/kg/day BVS-76D (XVII) treatment surpassed efficacy of levofloxacin at the same dose (100% survival versus 88.9% survival, respectively). Because all of the BVS-76D (XVII) treatment groups provided 100% protection, the ED50 is not able to be determined from these results.

### Anti-Tularemia Test Data

The protective efficacy of a range of BVS-76D (XVII) dosages against inhalational tularemia (F. tularensis) in BALB/c mice was evaluated.

Fifty naive female BALB/c mice were purchased from Jackson Laboratories for this study. The mice were 5-6 weeks old and the average weight was 19 grams. The animals were in good health, and free of malformations and clinical signs of disease. No animal developed an illness or injury unrelated to this study prior to the initial challenge or after treatment. Therefore, no replacement mice were added into the study. Upon arrival, the mice acclimated for several days prior to manipulation. During this time animals were marked for identification purposes and were randomized into the following treatment groups.

TABLE 8

| | | | | | | Challenge |
|---|---|---|---|---|---|---|
| | Group | Test Item Administration | | | | Administration |
| Group | Size | Description | Dose | Route | Schedule | Dose |
| 1 | 10 | Vehicle | 0.2% | i.p. | Once for 6 days | 3 LD₅₀ |
| | | control | | | beginning 24 h | |
| | | (DMSO) | | | post-challenge | |
| 2 | 10 | Levofloxacin | 10 mg/kg/ | i.p. | Once for 10 days | 3 LD₅₀ |
| | | | day | | beginning 24 h | |
| | | | | | post-challenge | |
| 3 | 10 | BVS-76D | 10 mg/kg/ | i.p. | Once for 6 days | 3 LD₅₀ |
| | | (XVII) | day | | beginning 24 h | |
| | | | | | post-challenge | |
| 4 | 10 | BVS-76D | 20 mg/kg/ | i.p. | Once for 6 days | 3 LD₅₀ |
| | | (XVII) | day | | beginning 24 h | |
| | | | | | post-challenge | |
| 5 | 10 | BVS-76D | 40 mg/kg/ | i.p. | Once for 6 days | 3 LD₅₀ |
| | | (XVII) | day | | beginning 24 h | |
| | | | | | post-challenge | |

F. tularensis SCHU S4 strain (3 LD₅₀) was administered intranasally to all animals in all groups on Day 0. Animals in Group 2 were treated with 10 mg/kg/day Levofloxacin once daily via the i.p. route for 10 consecutive days, beginning 24 hours post-infection. Animals in Groups 3-5 were treated with varying doses of BVS-76D (XVII) once daily for 6 consecutive days via the i.p. route, beginning 24 hours post-infection. The animals were evaluated for 13 days post-challenge. Survival and clinical signs of disease were recorded on a log sheet.

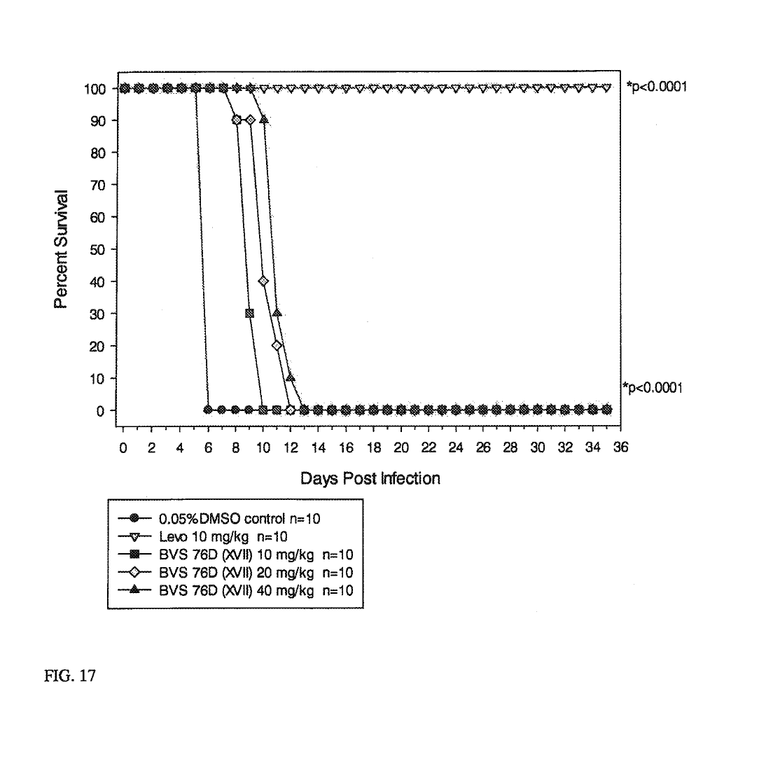

As the survival graph in FIG. 17 depicts, BVS-76D (XVII) provided partial protection to mice infected with 3 LD₅₀ F. tularensis, in a dose dependent manner but this protection was to not statistically significant when compared to Levofloxacin even at the highest dose of BVS-76D (XVII). All of the vehicle control animals that received 0.2% DMSO (Group 1) succumbed to infection by Day 6 post-challenge, thus representing a proper negative control group.

Levofloxacin (10 mg/kg/day) treatment resulted in 100% survival. However, all of the BVS-76D (XVII) doses (10-, 20- and 40 mg/kg/day) afforded partial protection to the animals by only extending the time to death in a dose dependent manner. Only the Levofloxacin treatment curve was statistically significant compared to the 0.2% DMSO control group, Group 1 (p<0.00006 as determined by Kaplan Meier Survival Estimates followed by Pairwise Multiple Comparison Procedures (Holm-Sidak method)).

Symptoms of all animals (Groups 1-5) were also documented throughout the study. Animals belonging to the Levofloxacin group, Group 2, never showed any outward indication of tularemia infection. However, animals in the DMSO control group (Group 1) showed ruffled fur on Day 4, 5 and died on day 6. By Day 5 post-infection, the DMSO control group (Group 1) appeared moribund and, as expected, showed typical signs of tularemia infection (i.e., ruffled fur, eye infection). Mice that received BVS-76 treatment showed ruffled fur on Days 7, 8 and 9 (Group 3), on days 8, 9, and 10 (Group 4) and on days 9 and 10 (Group 5) post-infection.

The first death in Group 3, one animal, occurred on day 8. The majority of deaths in this group occurred on day 9, with the remaining three animals dead by day 10. Although the first death in Group 4 occurred on day 7, most of the deaths occurred on day 10 and all the mice in this group were expired by day 12. The death rate of the highest treatment dose group, Group 5, appeared to lag those in Group 4 by one day. By day 13, all the mice in test Groups 3, 4 and 5 were dead. Overall, these data indicate that BVS-76D (XVII) is not significantly effective against inhalational tularemia in BALB/c mice as assessed by Kaplan Meier Survival Estimates followed by Pairwise Multiple Comparison Procedures). Analyzing the data with the less stringent statistical analysis method of the Kaplan-Meier Survival Estimates followed by the Chi-square test, each BVS-76D (XVII) curve of 10-, 20- and 40 mg/kg/day was significant when compared to the vehicle control curve. The 40 mg/kg/day BVS-76D (XVII) treatment was relatively effective compared to vehicle control as it extended the life span of the mice from 6 days to an average 10 days. The relatively short period of treatment (6 days) with BVS-76D (XVII) compared to Levofloxacin (10 days) may have affected the outcome.

BVS-60C(XVII), BVS-47A (XX), BVS-76D (XVII) and BVS-71A (XV) have MIC90 values below 2 micrograms/ml against all variants of B. pseudomallei tested. The range achieved by these compounds varied from 0.06 to 2 micrograms/ml. Against B. anthracis, BVS-60C (XVII), BVS-47A (XX), BVS-71A(XV) and BVS-76D (XVII) had MIC90 values of 0.25 micrograms/ml or lower. BVS-20A and BVS-97E (XIV) had MIC90 values between 2 and 4 micrograms/ml. Against F. Tularensis, BVS-20A, BVS-60C (XVII), BVS-47A (XX), BVS-76D (XVII) and BVS-71A (XV) had MIC90 levels below 4 micrograms/ml. For B. Mallei, BVS-71A (XV), BVS-60C (XVII), BVS-47A (XX), BVS-76D (XVII) and BVS-71A (XV) had MIC90 values below the 4 micrograms/ml threshold. For Y. pestis, BVS-60-C (XVII), BVS-47A (XX), BVS-76D (XVII) and BVS-71A (XV) had MIC90 values below 4 micrograms/ml.

### Anti-Mycobacterium Tuberculosis Test Data

TABLE 9
TB MIC Testing (range tested: 0.078 μg/ml to 10 μg/ml) against Single Resistant
Strains and the standard H37Rv Colorado strain and MBC Determination with H37Rv Colorado
strain only.

| | MIC Value (μg/ml) | | | | | | | |
|---|---|---|---|---|---|---|---|---|
| | | | | | | ATCC | | |
| | Standard | ATCC | ATCC | ATCC | ATCC | 35821 | ATCC | |
| | Test | 35837 | 35830 | 35827 | 35822 | Para- | 35820 | |
| | Strain | Ethambutol | Ethionamide | Kanamycin | Isoniazid | aminosalicylic | Streptomycin | MBC* |
| Compounds | H27Rv | Resistant | Resistant | Resistant | Resistant | Resistant | Resistant | (μg/ml) |
| BVS-60C | 0.312 | 0.625 | 0.312 | 0.625 | 0.625 | 0.625-1.25 | 0.312 | 0.625 |
| (XVII) | | | | | | | | |
| BVS-76D | 0.312-0.625 | 0.312-0.625 | 0.156 | 0.625 | 0.625 | 0.625-1.25 | 0.312 | 0.625 |
| (XVII) | | | | | | | | |
| BVS-85A | 1.25 | 1.25 | 0.625 | 1.25-2.5 | 1.25 | 2.5 | 0.625 | 1.25 |
| (XVII) | | | | | | | | |
| Rifampin | 0.0031 | Acceptable range: 0.0031 to 0.0125 μg/ml | | | | | | 0.0063 |
| Isoniazid | 0.031 | Acceptable range: 0.0031 to 0.125 μg/ml | | | | | | 0.031 |
| Antibiotic | NA | >8 | >8 | >8 | >8 | >8 | >8 | |
| Resistance | | | | | | | | |

*Performed with H37Rv. Typical range plated: MIC value and 2 dilutions above and below the MIC value.

TABLE 10
DMID TB MIC Results

| Compound | Solvent | Results (μg/ml) | IC50 | IC90 |
|---|---|---|---|---|
| BVS-60C (XVII) | DMSO | 0.312 | 0.22 | 0.23 |
| BVS-71A (XV) | DMSO | 2.5 | 1.38 | 1.55 |
| BVS-76D (XVII) | DMSO | 1.25 | 1.1 | 1.19 |
| BVS-85A (XVII) | DMSO | 0.625 | 0.53 | 0.57 |
| Rifampin | DMSO | 0.006 | | |
| Isoniazid | DMSO | 0.06 | | |

TABLE 11
Cytotoxicity Assay as Measured by CellTiter-Glo
Luminescent Cell Viability Assay using Vero Cells

| | % Viability* | | |
|---|---|---|---|
| Concentration | BVS-60C (XVII) | BVS-85A (XVII) | BVS-76D (XVII) |
| (μg/ml) | % | % | % |
| 20 | 96 | 107 | 96 |
| 10 | 88 | 101 | 100 |
| 5 | 92 | 106 | 113 |
| 2.5 | 102 | 109 | 111 |
| 1.25 | 107 | 107 | 131 |
| 0.625 | 87 | 105 | 112 |
| 0.312 | 100 | 104 | 103 |
| 0.156 | 104 | 114 | 122 |
| 0.078 | 103 | 108 | 114 |
| 0.039 | 101 | 108 | 112 |
| 0.02 | 95 | 109 | 105 |
| 0.01 | 93 | 109 | 119 |

*Assay performed in Vero cells. % viability as compared to 0.625% DMSO controls.

### Test Data Against A & B Bacterial Pathogens

A representative group of the compounds was investigated for in vitro activity against 30 strains of Bacillus anthracis, Burkholderia pseudomallei, Francisella tularensis, Burkholderia mallei and Yersinia pestis.

A four microgram/ml in vitro MIC90 value is typically considered to be a good candidate for in vivo studies, as this is a value that is considered to reasonably achievable in serum. The compounds had MIC90 values well below this level. In many cases, the compounds also demonstrated broad spectrum activity.

BVS-60C (XVII), BVS-47A (XX), BVS-76D (XVII), and BVS-71A (XV) have MIC90 values below 2 micrograms/ml against all variants of B. pseudomallei tested. The range to achieved by these compounds varied from 0.06 to 2 micrograms/ml.

Against B. anthracis, it was found that BVS-60C (XVII), BVS-47A (XX), BVS-71A (XV), and BVS-76D (XVII) have MIC90 values of 0.25 micrograms/ml or lower. BVS-20A, and BVS-97E (XIV) had MIC90 values between 2 and 4 micrograms/ml.

Against F. Tularensis, it was found that BVS-20A, BVS-60C (XVII), BVS-47A (XX), BVS-76D (XVII) and BVS-71A (XV) have MIC90 values below 4 micrograms/ml.

Against B. Mallei, it was found that BVS-71A (XV), BVS-60C (XVII), BVS-47A (XX), BVS-76D (XVII), and BVS-71A (XV) have MIC90 values below 4 micrograms/ml.

Against Y. pestis, it was found that BVS-60-C (XVII), BVS-47A (XX), BVS-76D (XVII), and BVS-71A (XV) have MIC90 values below 4 micrograms/ml.

In many cases, the MIC90 values were exceptionally low. The highest MIC90 value determined against these pathogens with these compounds was no higher than 2 micrograms/ml and this was against the more difficult to treat Burkholderia species.

BVS-20A shows good efficacy against B. anthracis and F. tularensis, with MIC90 values at 4 or less.

It was surprisingly and unexpectedly found that these compounds are equally effective against all of the tested pathogens. The inventive compounds appear to have overcome the difficulties experienced by other classes of antimicrobials, which are typically only effective against a single or a few pathogens.

A representative group of the compounds was also tested against B. anthracis, F. tularensis, Y. pestis, Burkholderia mallei, Burkholderia pseudomallei, E. coli and P. aeruginosa in triplicate. All of the compounds showed activity against several or all of the organisms. The test panel for MIC evaluation included Quality Control (QC) strains E. coli and P. aeruginosa recommended by CLSI and a panel of five biothreat agents. Standardized cell suspensions were prepared and inoculated into the plates. After incubation, the MIC was determined as described below.

Test compounds were dissolved in sterile DMSO at a stock concentration of 1280 μg/ml. A total of eleven dilutions were then made in their respective media. Compounds were tested against B. anthracis, B. mallei, B. pseudomallei, F. tularensis, and Y. pestis in a dose range between 0.06 and 64 μg/ml (concentration in well after inoculation).

Ciprofloxacin was the positive control antibiotic used for B. anthracis, B. mallei, B. pseudomallei, F. tularensis, and Y. pestis. E. coli and P. aeruginosa were the control strains.

All assays were performed using broth micro-dilution, following CLSI guidelines: M7-A7 Vol. 26, January 2006 and M100-S18 Vol 28, January 2008, using 96-well plates with a final volume of 200 μl/well. The compounds were tested in triplicate at 11 concentrations; dose range was 0.06 and 64 μg/ml of novel compound. Controls for media sterility, culture controls, and reagent sterility were also included in the assay. Broths were made to an OD620m of 0.08-0.12, it was then diluted to 1/150 for inoculation into the wells 100 μl of the different dilutions of the test compounds was added to each well, and 100 μl of growth medium containing the organism.

All strains were incubated at 37° C. After 24 hrs or 48 hrs incubation, the plates were read in a Multiskan at OD620 nm for growth, and data analyzed in Microsoft Excel 2007. The lowest concentration of test compound that inhibits growth is the MIC value for that replicate of the test compound.

TABLE 12
Test panel for MIC evaluation of five biothreat agents and Quality Control (QC)
strains E. coli and P. aeruginosa.

| | | | | F. tularensis | F. tularensis | | | |
|---|---|---|---|---|---|---|---|---|
| | | | | (Original | (Repeated | | | |
| | B. anthracis | B. mallei | B. pseudomallei | Test) | test) | Y. pestis | E. coli | P. aeruginosa |
| Compound | (μg/ml) | (μg/ml) | (μg/ml) | (μg/ml) | (μg/ml) | (μg/ml) | (μg/ml) | (μg/ml) |
| BVS-71A | <0.06 | 0.25 | 2-4 | <0.06 | NG | <0.06 | <0.06 | 0.25 |
| (XV) | | | | | | | | |
| BVS-85A | <0.06 | 0.5 | 8 | <0.06 | NG | <0.06-0.12 | <0.06 | 0.5 |
| (XVII) | | | | | | | | |
| BVS-60C | <0.06 | 1 | 1-2 | <0.06 | NG | 0.06 | 0.06-0.12 | 2 |
| (XVII) | | | | | | | | |
| BVS-97E | 4 | >64 | >64 | 0.5-4 | NG | >64 | 16-32 | >64 |
| (XIV) | | | | | | | | |
| BVS-76D | <0.06 | 1 | 2-4 | <0.06 | NG | <0.06 | <0.06 | 0.25 |
| (XVII) | | | | | | | | |
| ciprofloxacin | <0.03 | 1 | 0.5-1 | 0.015-0.06 | NG | 0.015-0.03 | 0.015-0.06 | 0.25 |

P. aeruginosa is included due to not being able to repeat the tests.
F. tularensis is included as the repeat tests had no growth, therefore there are no results.

TABLE 13
ANTI-MALARIA TESTING

| | IC50 (nm) | IC50 (nm) | Average |
|---|---|---|---|
| Compound | Test 1 | Test 2 | IC50 (nm) |
| BVS-76D (XVII) | 4019 | 1429 | 2724 |
| BVS-85A (XVII) | 5453 | 9059 | 7256 |
| BVS-47A (V) | >10000 | >10000 | >10000 |
| BVS-60C (XVII) | 6107 | No data | 6107 |

### Anti-Influenza Testing

BVS-20A (XII), BVS-45A (I), and BVS-47A (V), shown below, were found to be highly active by visual and NR assays against Flu A, H1N1 and Flu A H3N2, viral strains New Caledonia/20/99 and California/7/04. The results of the testing are shown in Table 14 below,

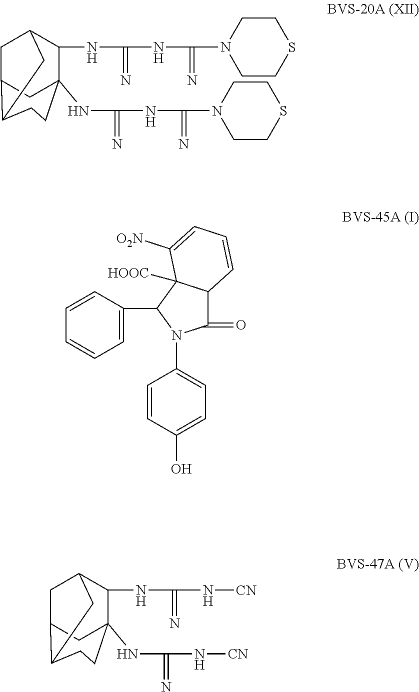 [SMILES: N=C(NC(=N)N1CCSCC1)NC1C2CC3CCC1(NC(=N)NC(=N)N1CCSCC1)CC3C2 ; O=C1C2C=CC=C([N+](=O)[O-])C2(C(=O)O)C(c2ccccc2)N1c1ccc(O)cc1 ; N#CNC(=N)NC1C2CC3CCC1(NC(=N)NC#N)CC3C2]

TABLE 13
Anti-influenza Testing Results

| Cmpd | | | | | | Drug | Test | | | | | |
|---|---|---|---|---|---|---|---|---|---|---|---|---|
| Name | Assay | Vehicle | Virus | Virus Strain | Cell Line | Unit | Date | Trial # | EC50 | EC90 | IC50 | Si |
| BVS-45 | Neutral | DMSO | Flu A | New Caledonia/20/99 | MDCK | μg/ml | Sep. 21, 2007 | 1 | 0.88 | | >100 | >110 |
| | Red | | (H1N1) | | | | | | | | | |
| | Visual | DMSO | Flu A | New Caledonia/20/99 | MDCK | μg/ml | Sep. 21, 2007 | 1 | 0.18 | | >100 | >580 |
| | | | (H1N1) | | | | | | | | | |
| | Virus | DMSO | Flu A | New Caledonia/20/99 | MDCK | μg/ml | Sep. 21, 2007 | 2 | | >100 | | 0 |
| | Yield | | (H1N1) | | | | | | | | | |
| | Visual- | DMSO | Flu A | New Caledonia/20/99 | MDCK | μg/ml | Sep. 21, 2007 | 2 | 0.18 | | >100 | >560 |
| | CONF | | (H1N1) | | | | | | | | | |
| | Neutral | DMSO | Flu A | California/7/04 | MDCK | μg/ml | Sep. 21, 2007 | 1 | 0.22 | | 75 | 340 |
| | Red | | (H3N2) | | | | | | | | | |
| | Visual | DMSO | Flu A | California/7/04 | MDCK | μg/ml | Sep. 21, 2007 | 1 | 0.056 | | >100 | >1786 |
| | | | (H3N2) | | | | | | | | | |
| | Virus | DMSO | Flu A | California/7/04 | MDCK | μg/ml | Sep. 21, 2007 | 2 | | 0.15 | | >667 |
| | Yield | | (H3N2) | | | | | | | | | |
| | Visual- | DMSO | Flu A | California/7/04 | MDCK | μg/ml | Sep. 21, 2007 | 2 | 0.056 | | >100 | >1786 |
| | CONF | | (H3N2) | | | | | | | | | |
| BVS-47 | Neutral | DMSO | Flu A | New Caledonia/20/99 | MDCK | μg/ml | Sep. 21, 2007 | 1 | 0.25 | | 70 | >280 |
| | Red | | (H1N1) | | | | | | | | | |
| | Visual | DMSO | Flu A | New Caledonia/20/99 | MDCK | μg/ml | Sep. 21, 2007 | 1 | 0.075 | | >100 | >1333 |
| | | | (H1N1) | | | | | | | | | |
| | Virus | DMSO | Flu A | New Caledonia/20/99 | MDCK | μg/ml | Sep. 21, 2007 | 2 | | 40 | | >2.5 |
| | Yield | | (H1N1) | | | | | | | | | |
| | Visual- | DMSO | Flu A | New Caledonia/20/99 | MDCK | μg/ml | Sep. 21, 2007 | 2 | 0.075 | | >100 | >1333 |
| | CONF | | (H1N1) | | | | | | | | | |
| | Neutral | DMSO | Flu A | California/7/04 | MDCK | μg/ml | Sep. 21, 2007 | 1 | 0.72 | | 79 | 110 |
| | Red | | (H3N2) | | | | | | | | | |
| | Visual | DMSO | Flu A | California/7/04 | MDCK | μg/ml | Sep. 21, 2007 | 1 | 0.18 | | >100 | >560 |
| | | | (H3N2) | | | | | | | | | |
| | Virus | DMSO | Flu A | California/7/04 | MDCK | μg/ml | Sep. 21, 2007 | 2 | | 3.2 | | >31 |
| | Yield | | (H3N2) | | | | | | | | | |
| | Visual- | DMSO | Flu A | California/7/04 | MDCK | μg/ml | Sep. 21, 2007 | 2 | 0.18 | | >100 | >560 |
| | CONF | | (H3N2) | | | | | | | | | |

ANTI_BACTERIAL ACTIVITY TEST DATA OF COMPOUND BVS-20A (XII)
MIC END POINT (mg/ml)

| S. aureus | P. aeruginosa | S. marcescens | C. albicans | F. solani |
|---|---|---|---|---|
| 0.008 | 0.065 | ≧1.04 | 0.065 | 0.52 |

STAND ALONE BIOCIDAL RESULTS OF COMPOUND BVS-20A (XII)

| Concentration | | Staphylococcus | Pseudomonas | Serratia | Candida | Fusarium |
|---|---|---|---|---|---|---|
| (ppm) | Time | aureus | aeruginosa | marcescens | albicans | Solani |
| 1000 | 4 hr | 2.4 | >4.7 | 3.8 | >4.7 | >4.4 |
| 333 | | 1.8 | >4.7 | 2 | 4.2 | >4,4 |
| 111 | | 1.5 | 3.6 | 1.2 | 0.9 | 2.4 |
| 37 | | 1.5 | 3.1 | 1.2 | 1 | 1.5 |
| 12.3 | | 1.3 | 1.9 | 2.1 | 0.8 | 0.7 |
| 400 | | 2.4 | 3.5 | 2.2 | 4.1 | 3.8 |
| 300 | | 2.1 | 3.4 | 1.9 | 2 | 2.4 |
| 200 | | 1.8 | 3.2 | 1.9 | 2.5 | 2.5 |
| 100 | | 1.6 | 3 | 1.7 | 1.3 | 1.7 |

### Ocular Disease Treatment

Fluoroquinolones were found to be safe and to have outstanding penetration, low sensitization rate and excellent safety profiles for the treatment of ocular diseases in general and bacterial infections caused by gram-positive and gram-negative organisms in particular. The quinolone-azaquinolone carboxylic acid antibacterial drugs have been widely used as therapeutic agents to treat wide spectrum bacterial infections. The fluoroquinolone compounds disclosed herein act not only against bacteria but also against viruses. These additional characteristics have surprisingly potent beneficial effects in treating bacterial and viral conjunctivitis.

The mode of action of fluoroquinolones is through the inhibition of bacterial DNA gyrase. This essential enzyme controls DNA topology and assist in DNA replications, repairs, decatenation and transcriptions. In contrast to the bacterial enzymes, mammalian topoisomerase II is highly resistant to inhibition by quinolones and azaquinolones. The bacterial gyrase is approximately 100 times more sensitive to inhibition than eukaryotic equivalent.

The previously existing antibiotic of choice to treat bacterial conjunctivitis is fluoroquinolones (quinolones). They are effective, penetrate the ocular tissue, safe and comfortable to use. They have broad-spectrum anti-bacterial activity and low rates of microbial resistance. Numerous studies have demonstrated that fluoroquinolones effectively kill a wide spectrum of ocular pathogens. Several drugs from this class of fluoroquinolones have been widely used to treat ocular infections. For example, the second generation quinolones-ciprofloxacin (Ciloxan®, Alcon), (Besivance®, Bausch & Lomb) and ofloxacin (Ocuflox®, Allergan), the third generation quinolone-levofloxacin (Quixin®, Santen) and the fourth generation quinolones-gatifloxacin (Zymar®, Allergan) and moxifloxacin (Vigamox®, Alcon) are widely used at present. They have greater overall in vitro efficacy than gentamicin, chloramphenicol, tobramycin, tetracycline and erythromycin. Ciprofloxacin has been shown to be effective against external ocular infection. It was found to be safe and effective in children with bacterial conjunctivitis for local use. Ofloxacin has excellent penetration into ocular tissues, cornea, aqueous humor and vitreous humor. Both ciprofloxacin and ofloxacin are therapeutics of choice as they have demonstrated excellent efficacy, penetration, and comfort and safety profile.

Fluoroquinolones are safe, and effective in pediatric use. They are better tolerated than other drugs, have potent antimicrobial activity against a broad spectrum of ocular pathogens and greater penetration into ocular tissues. Their superior penetrations provide an increased eradication and decreased likelihood of infection-recurrence.

Fluoroquinolones inhibit the development of resistance by binding with nuclear enzymes of bacteria. The second and third generation quinolones prevent transcription, replication and coiling of bacterial DNA by inhibiting DNA gyrase in gram-negative organisms and topoisomerase IV in gram-positive organisms. The likelihood of resistance developing is one in 10 million. Fluoroquinolones are effective in enabling resistance because they attack both DNA gyrase and topoisomerase IV in gram-positive organisms. Bacterial resistance is rare because two spontaneous mutations of the bacterial DNA would be necessary for resistance to develop and hence the likelihood of resistance of one in trillion.

The pathogens of conjunctivitis vary but bacteria are the most common cause. The second most common cause of conjunctivitis is a virus like adenovirus. Bacterial infections caused by gram-positive pathogens are more predominant than those caused by gram-negative pathogens.

It has been found that the compounds disclosed herein have the additional benefit of not only killing gram negative and gram positive bacteria, but also viruses. Hence, they have broader spectrum of activity than prior fluoroquinolones. In some embodiments, the invention includes a method of treating ocular diseases by administering to a patient suffering from one or more ocular disease, a composition that includes one or more of the compounds disclosed in this application.

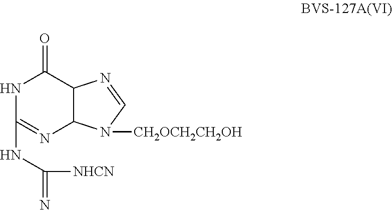

BVS-127A(VI) was found to be particularly effective for treating ocular diseases, Hepatitis C Virus (HCV) and HIV-1.

The following prodrug of BVS-127A (VI) is particularly useful according to the invention:

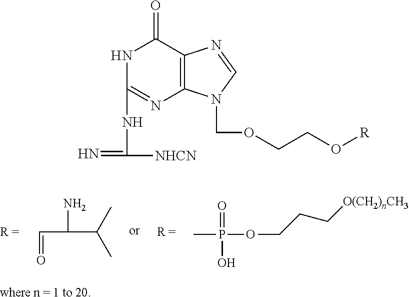

The prodrug is an esterified version of BVS-127A (VI) that has greater oral bioavailability, It is converted by esterase to active drug, BVS-127A (VI) via hepatic first-phase metabolism. The prodrug is particularly useful according to the invention to increase efficacy by boosting intracellular levels of active drug and decrease nephrotoxicity, especially against HIV-1.

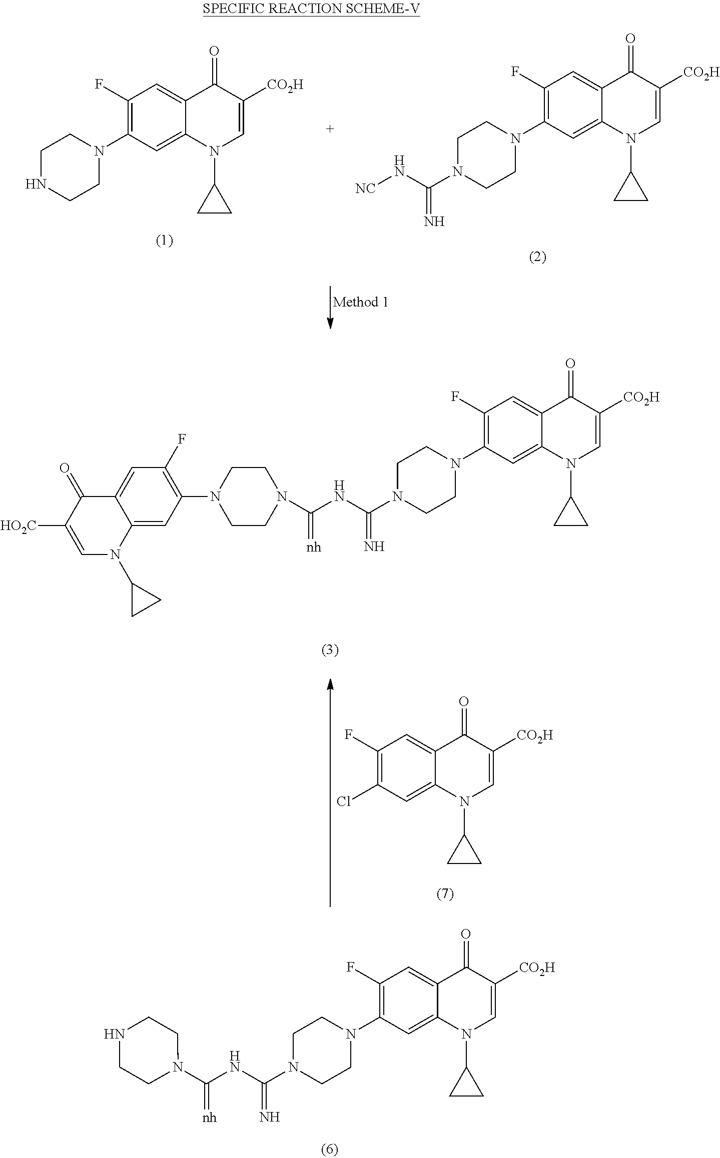

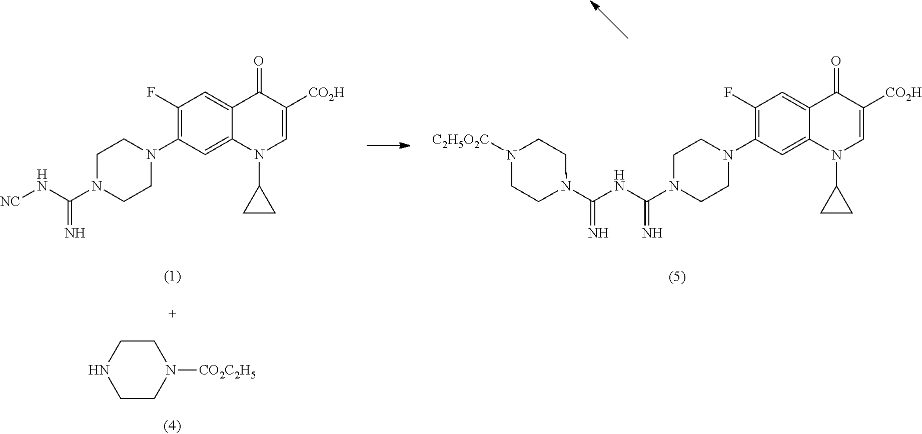

### Method-1.

A mixture of 1-cyclopropyl-6-fluoro-1,4-dihydro-4-oxo-7-(1-piperazinyl)-quinoline carboxylic acid hydrochloride (1) (2.5 g), 1-cyclopropyl-6-fluoro-1,4-dihydro-4-oxo-7-(4-cyanoguanidine-1-piperazinyl)quinoline carboxylic acid (2) (3 g) and a few drops of HCl was finely ground and heated at 285-290° C. for 1 hr. The crude solid was mixed with methanol, acidified with HCl, heated to boiling, decolorized with charcoal, filtered, concentrated to a small volume under reduced pressure, cooled on dry ice and the resulting solid was removed by filtration. The process was repeated two more times. wt. 0.7 g (3) m.p. 300-305° C. Anal. Calcd for: C₃₆H₄₁N₉F₂Cl₄O₆.H₂O.

| | C | H | N | F | Cl |
|---|---|---|---|---|---|
| Theory: | 48.36 | 4.85 | 14.10 | 4.25 | 15.86 |
| Found: | 48.44 | 4.96 | 14.85 | 3.18 | 14.01 |

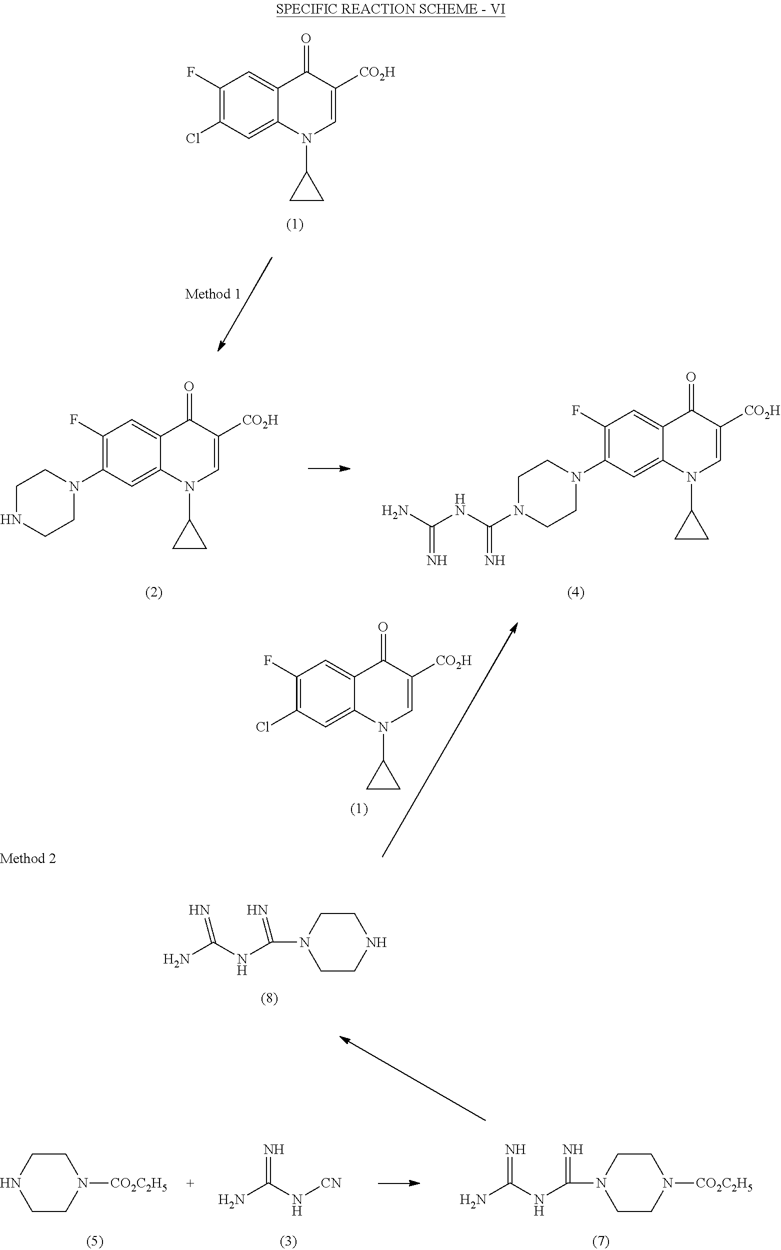

### 1-Cyclopropyl-6-fluoro-1,4-dihydro-4-oxo-7-(1-piperazinyl-4-(3-amino biguanidine)-quinoline carboxylic acid dihydrochloride (4)

### Method-1

A mixture of 1-cyclopropyl-6-fluoro-1,4-dihydro-4-oxo-7-(1-piperazinyl)-quinoline carboxylic acid hydrochloride (2) (5 g), n-butanol (100 mL) and dicyandiamide (1.5 g) was refluxed with stirring for 23 hours. It was evaporated to dryness under reduced pressure. The crude solid was mixed with water, filtered and washed with water. The resulting solid was mixed with ethanol, acidified with HCl, evaporated to dryness, dissolved in water, filtered, concentrated to a small volume, cooled and filtered to yield 1.3 g (4). m.p.=168-172° C. Anal. Calcd. for: C₁₉H₂₄N₇FCl₂O₃

| | C | H | N | F | Cl |
|---|---|---|---|---|---|
| Theory: | 46.73 | 4.95 | 20.06 | 3.89 | 14.72 |
| Found: | 45.77 | 5.13 | 19.38 | 3.68 | 14.60 |

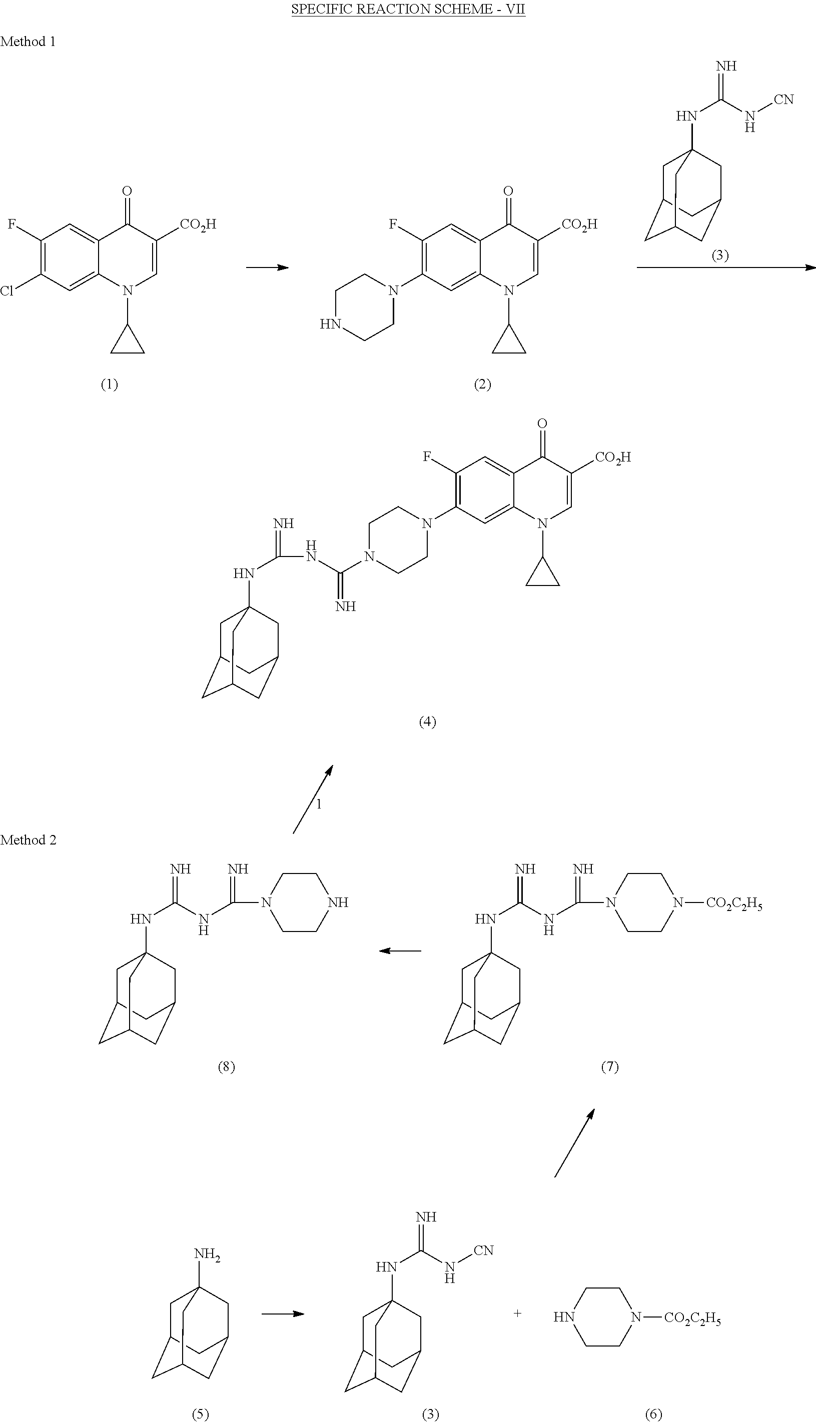

### 1-Cyano guanidino adamantanamine (3)

### Method-1

A mixture of 1-adamantanamine hydrochloride (100 g), sodium dicyanamide (55 g), n-butanol (1 L) and water (84 mL) was refluxed with stirring for 27 h. It was evaporated to dryness, mixed with water, made alkaline with NaOH, filtered, washed with water and acetone. The resulting crude solid was recrystallized from an equal mixture of boiling methanol and ethanol to yield 40 g of (3) m.p.=295-300° C.

Anal. Calcd. for: C₁₂H₁₈N₄

| | C | H | N |
|---|---|---|---|
| Theory: | 66.02 | 8.31 | 25.66 |
| Found: | 65.95 | 8.34 | 25.58 |

### 1-Cyclopropyl-6-fluoro-1,4-dihydro-4-oxo-7-(1-piperazinyl-4-(3-(1-adamantanamino) biguanidine) quinoline carboxylic acid hydrochloride (4)

A mixture of 1-cyclopropyl-6-fluoro-1,4-dihydro-4-oxo-7-(1-piperazinyl)-quinoline carboxylic hydrochloride (2) (5 g), 1-cyanoguanidine adamantanamine (3) (2.9 g) and n-butanol (200 mL) was refluxed with stirring for 174 h. It was evaporated to dryness under reduced pressure. The resulting crude solid was recrystallized twice from a mixture of boiling THF and water to give 1.5 g of (4) m.p.=260° C.-265° C.

Anal. Calcd. for: C₂₉H₃₇N₇FClO₃

| | C | H | N | F | Cl |
|---|---|---|---|---|---|
| Theory: | 59.43 | 6.36 | 16.72 | 3.24 | 6.04 |
| Found: | 58.06 | 6.77 | 16.11 | 2.75 | 5.70 |

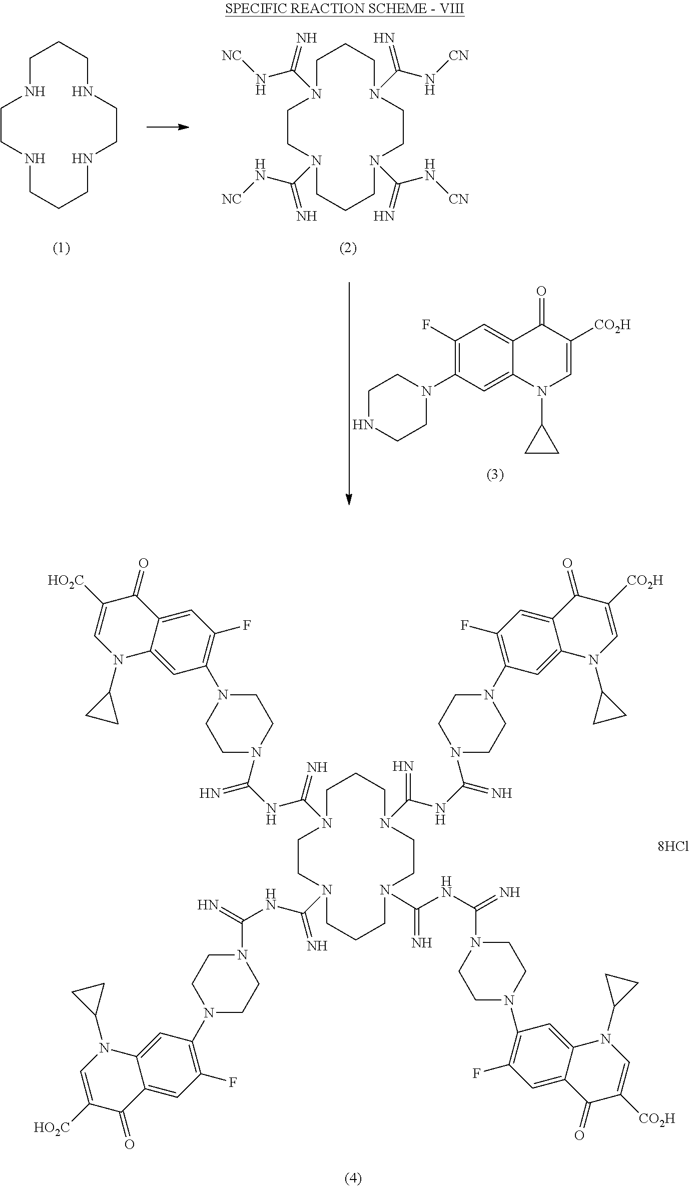 [SMILES: C1CNCCNCCCNCCNC1 ; N#CNC(=N)N1CCCN(C(=N)NC#N)CCN(C(=N)NC#N)CCCN(C(=N)NC#N)CC1 ; O=C(O)c1cn(C2CC2)c2cc(N3CCNCC3)c(F)cc2c1=O ; N=C(NC(=N)N1CCN(c2cc3c(cc2F)c(=O)c(C(=O)O)cn3C2CC2)CC1)N1CCCN(C(=N)NC(=N)N2CCN(c3cc4c(cc3F)c(=O)c(C(=O)O)cn4C3CC3)CC2)CCN(C(=N)NC(=N)N2CCN(c3cc4c(cc3F)c(=O)c(C(=O)O)cn4C3CC3)CC2)CCCN(C(=N)NC(=N)N2CCN(c3cc4c(cc3F)c(=O)c(C(=O)O)cn4C3CC3)CC2)CC1]

### 1,4,8,11-Tetraazacyclo-(1,4,8,11-tetra cyanoguanidino)-tetradecane (2)

1,4,8,11-tetraazacyclotetradecane (cyclam) (1) (5 g), sodium dicyandiamide (8.8 g), n-butanol (150 mL) and water (12 mL) were mixed with 1 mL of HCl. The mixture was refluxed with stirring for 25 h. It was evaporated under reduced pressure and the residue was mixed with water, acidified with HCl, filtered, washed with water and acetone. The waxy solid mixed with water again, basified with NaOH, filtered, washed with water and acetone to give 1.3 g of (2).

m.p.=>300° C.

Anal. Calcd. for: C₁₈H₂₈N₆

| | C | H | N |
|---|---|---|---|
| Theory: | 46.14 | 6.02 | 47.83 |
| Found: | 45.60 | 5.80 | 46.95 |

A mixture of (2) (1.3 g) and (3) (3.6 g) with a few drops of HCl was fused at 330-35° C. for 1 h. It was cooled, dissolved in boiling methanol (400 mL), decolorized with activated charcoal, filtered and evaporated under reduced pressure. The process was repeated again twice to yield 0.1 g of (4).

m.p.=>300° C.

Anal. Calcd. for: C₈₆H₁₀₈N₂₈F₄Cl₈O₁₂

| | C | H | N | F | Cl |
|---|---|---|---|---|---|
| Theory: | 49.52 | 5.21 | 18.80 | 3.64 | 13.59 |
| Found: | 49.54 | 5.44 | 18.04 | 3.16 | 11.50 |

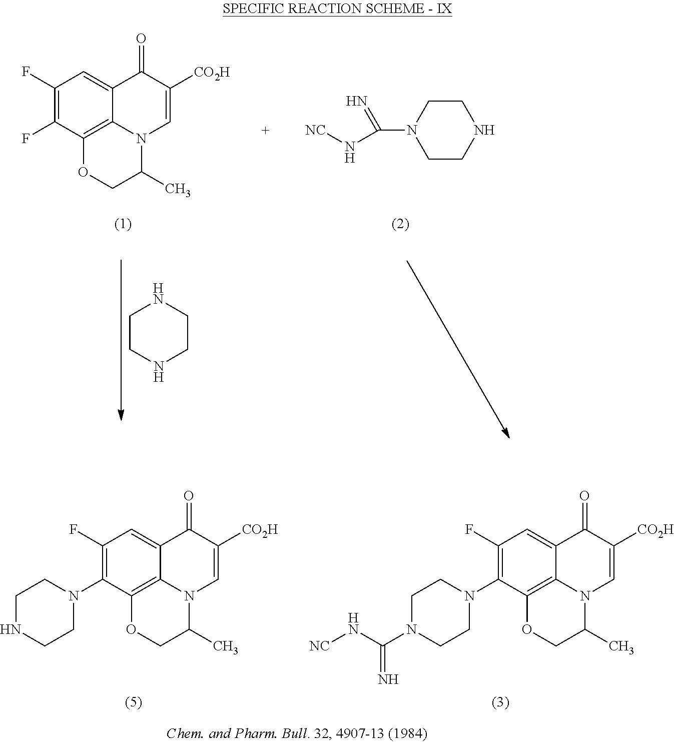 [SMILES: CC1COc2c(F)c(F)cc3c(=O)c(C(=O)O)cn1c23 ; N#CNC(=N)N1CCNCC1 ; C1CNCCN1 ; CC1COc2c(N3CCNCC3)c(F)cc3c(=O)c(C(=O)O)cn1c23 ; CC1COc2c(N3CCN(C(=N)NC#N)CC3)c(F)cc3c(=O)c(C(=O)O)cn1c23]

### (±)-9-fluoro-2,3-dihydro-3-methyl-10-(1-piperazinyl)-7-oxo-7H-pyrido[1,2,3-de]-1,4-benzoxazine-6-carboxylic acid hydrochloride (5)

### Method-2

A mixture of 9,10-difluoro-3-methyl-7-oxo-2,3-dihydro-7H-pyrido-(1,2,3-de)-(1,4)benzoxazine-6-carboxylic acid (20 g) (1), anhydrous piperazine (22.96 g) and pyridine (220 mL) was refluxed with stirring for 7 h. It was evaporated to dryness, mixed with water, filtered and dried. The crude solid was mixed with methanol and water, acidified with HCl, heated to boiling, filtered and cooled to yield 16 g. of (5) (±)-9-fluoro-2,3-dihydro-3-methyl-10-(1-piperazinyl)-7-oxo-7H-pyrido[1,2,3-de]-1,4-benzoxazine-6-carboxylic acid hydrochloride (5). wt.=11 g. m.p.>300° C.

Anal. Calcd. for: C₁₇H₁₉N₃FClO₄

| | C | H | N | F | Cl |
|---|---|---|---|---|---|
| Theory: | 53.19 | 4.98 | 10.94 | 4.95 | 9.23 |
| Found: | 52.76 | 5.41 | 10.57 | 4.31 | 8.65 |

### (±)-9-Fluoro-2,3-dihydro-3-methyl-10-(4-cyanoguanidino-1-piperazinyl)-7-oxo-7H-pyrido[1,2,3-de]-1,4-benzoxazine-6-carboxylic acid (3)

A mixture of (5) (10 g), sodium dicyandiamide (3.5 g), n-butanol (250 mL), water (20 mL) and a few drops of HCl was refluxed with stirring for 25 h. It was evaporated to dryness, mixed with water (250 mL), filtered and washed with acetone. The solid was mixed with water again, made basic with NaOH, filtered, washed with water and acetone to yield (3). wt=4.3 g;

m.p.=298-300° C.

Anal. Calcd for: C₁₉H₁₉N₆FO₄

| | C | H | N | F |
|---|---|---|---|---|
| Theory: | 55.07 | 4.62 | 20.28 | 4.58 |
| Found: | 54.19 | 4.73 | 19.84 | 4.17 |

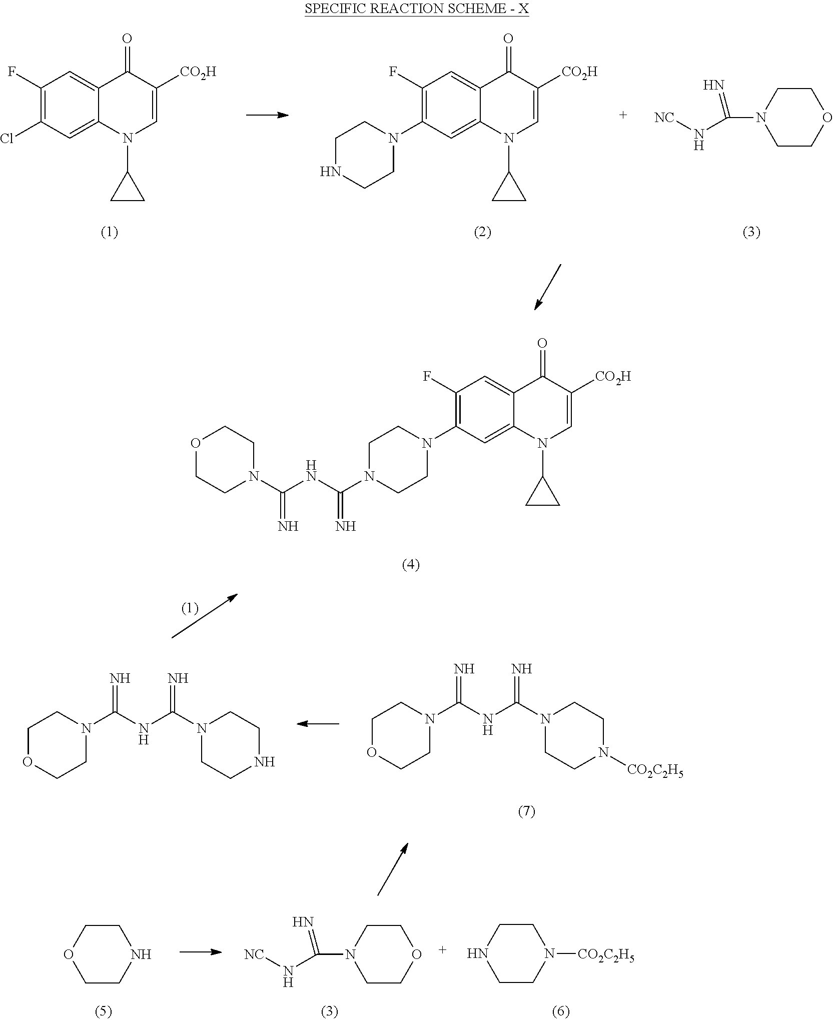

### 4-Cyanoguanidino-1-morpholine (3)

A solution of morpholine (50 mL) and 1-butanol (400 mL) was cooled and acidified with HCl and sodium dicyanamide (51.4 g) was added to it. The mixture was refluxed with stirring for 27 h and evaporated to dryness. The solid was mixed with water, made basic with sodium hydroxide, filtered, washed with water and recrystallized from boiling methanol to yield 37.8 g of (3). m.p.=225° C.-228° C.

Anal. Calcd. for: C₆H₁₀N₄O

| | C | H | N |
|---|---|---|---|
| Theory: | 46.74 | 6.53 | 36.33 |
| Found: | 46.79 | 6.54 | 36.30 |

### 1-Cyclopropyl-6-fluoro-1,4-dihydro-4-oxo-7-(1-piperazinyl)-4-guanidino-4′-morpholine)-quinoline carboxylic acid dihydrochloride (4)

A mixture of 1-cyclopropyl-6-fluoro-1,4-dihydro-4-oxo-7-(1-piperazinyl)-quinoline carboxylic acid (2) (2 g), 4-cyanoguanidine-1-morpholine (3) (0.93 g) and a few drops of HCl was triturated and fused at 235-240° C. for 2 h. It was cooled, dissolved in boiling methanol, acidified with HCl, decolorized with charcoal, filtered cooled and made turbid with acetone. It was filtered to yield 0.6 g of (4). m.p.=250-255° C.

Anal. Calcd. for: C₂₃H₃₀N₇FCl₂O₄

| | C | H | N | F | Cl |
|---|---|---|---|---|---|
| Theory: | 49.47 | 5.40 | 17.55 | 3.40 | 12.69 |
| Found: | 49.23 | 5.65 | 16.96 | 2.96 | 11.72 |

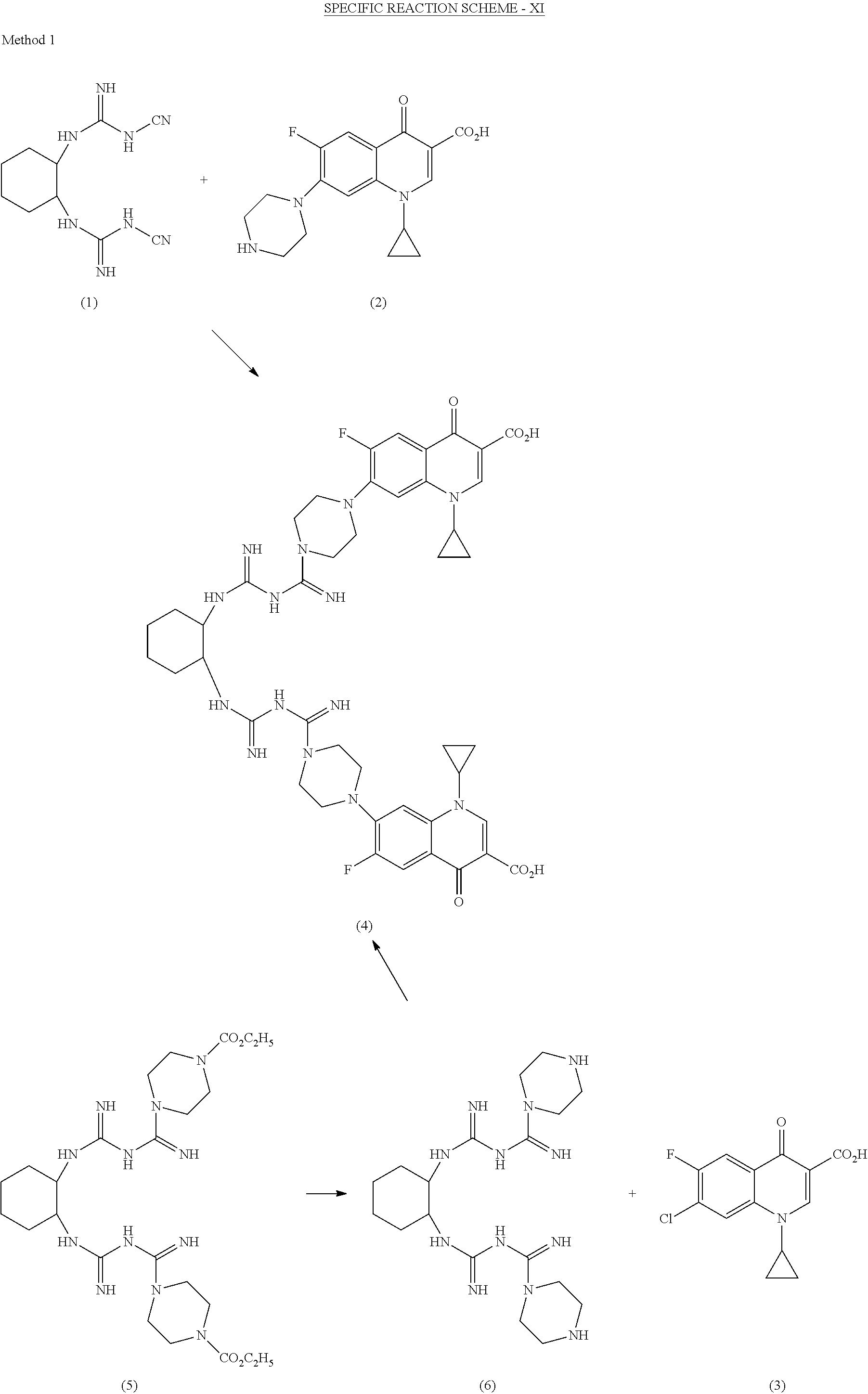 [SMILES: N#CNC(=N)NC1CCCCC1NC(=N)NC#N ; O=C(O)c1cn(C2CC2)c2cc(N3CCNCC3)c(F)cc2c1=O ; N=C(NC(=N)N1CCN(c2cc3c(cc2F)c(=O)c(C(=O)O)cn3C2CC2)CC1)NC1CCCCC1NC(=N)NC(=N)N1CCN(c2cc3c(cc2F)c(=O)c(C(=O)O)cn3C2CC2)CC1 ; CCOC(=O)N1CCN(C(=N)NC(=N)NC2CCCCC2NC(=N)NC(=N)N2CCN(C(=O)OCC)CC2)CC1 ; N=C(NC(=N)N1CCNCC1)NC1CCCCC1NC(=N)NC(=N)N1CCNCC1 ; O=C(O)c1cn(C2CC2)c2cc(Cl)c(F)cc2c1=O]

### 1,2-Dicyanodiguanidino cyclohexane (1)

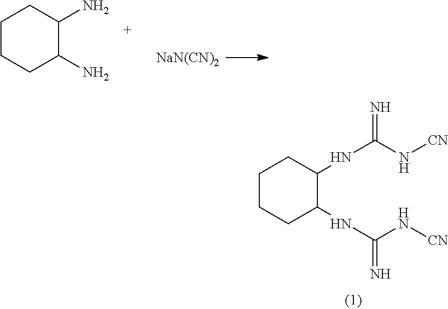

### Method-1

A mixture of 1,2-diaminocyclohexane (30.0 g), sodium dicyanamide (46.7 g), n-butanol (400 mL) and concentrated hydrochloric acid (5 mL) was refluxed with stirring for 28 hours. It was cooled, filtered and washed with acetone. The solid was mixed with about 250 cc water, acidified with cone-HCl, filtered, washed with water and acetone. Again the solid was mixed with about 300 cc water, made basic with 10% sodium hydroxide solution, filtered, washed with water and acetone. The process was repeated twice more and dried at room temperature. It was recrystallized from boiling DMF (450 mL) and water to give 13.5 g (1). m.p.=290-292° C.

Anal. Calcd. for: C₁₀H₁₆N₈

| | C | H | N |
|---|---|---|---|
| Theory: | 48.37 | 6.49 | 45.13 |
| Found: | 48.12 | 6.80 | 44.85 |

A mixture of 1,2-dicyanoguanidine cyclohexane (1.25 g) (1), 1-cyclopropyl-6-fluoro-1,4-dihydro-4-oxo-7-(1-piperazinyl)-3-quinolinecarboxylic acid (3.7 g) (2) and a few drops of conc. hydrochloric acid were mixed and fused with stirring at 285-290° C. for one hour. It was recrystallized from boiling ethanol and again from methanol and acetone. m.p.=285-290° C. wt.=0.5 g (4).

Anal. Calcd. for: C₄₄H₅₆H₁₄F₂Cl₄O₆.H₂O

| | C | H | N | F | Cl |
|---|---|---|---|---|---|
| Theory: | 49.16 | 5.49 | 18.24 | 3.53 | 13.00 |
| Found: | 49.56 | 5.96 | 18.12 | 2.58 | 12.90 |

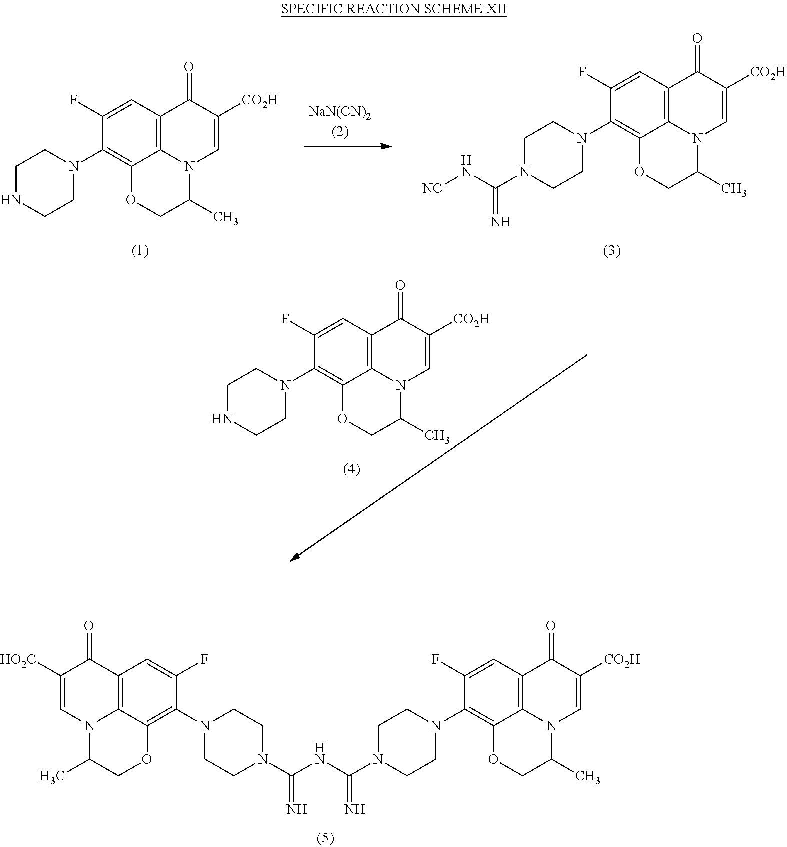 [SMILES: CC1COc2c(N3CCNCC3)c(F)cc3c(=O)c(C(=O)O)cn1c23 ; CC1COc2c(N3CCN(C(=N)NC#N)CC3)c(F)cc3c(=O)c(C(=O)O)cn1c23 ; CC1COc2c(N3CCNCC3)c(F)cc3c(=O)c(C(=O)O)cn1c23 ; CC1COc2c(N3CCN(C(=N)NC(=N)N4CCN(c5c(F)cc6c(=O)c(C(=O)O)cn7c6c5OCC7C)CC4)CC3)c(F)cc3c(=O)c(C(=O)O)cn1c23]

### Method-1

A mixture of (±)-9-fluoro-2,3-dihydro-3-methyl-10-(1-piperazinyl)-7-oxo-7H-pyrido[1,2,3-de]-1,4-benzoxazine-6-carboxylic acid (10 g) (1), sodium dicyanamid (2.3 g) (2), n-butanol (250 mL), water (20 mL) and cone HCl (1 mL) was refluxed with stirring 25 hours. The suspension was cooled and filtered. The resulting solid was mixed with water, made basic with sodium carbonate solution, filtered, washed with water and acetone. It was recrystallized from a boiling mixture of methanol and water.

wt. 4.3 g (3), m.p. 290-293° C.

Anal. Calcd. for: C₁₉H₁₉N₆FO₄

| | C | H | N | F |
|---|---|---|---|---|
| Theory: | 55.07 | 4.62 | 20.28 | 4.58 |
| Found: | 54.09 | 4.73 | 19.84 | 4.17 |

### Method-2

A mixture of (±)-9-fluoro-2,3-dihydro-3-methyl-10-(1-piperazinyl-4-cyanoguanidine)-7-oxo-7H-pyrido-[1,2,3-de]-1,4-benzoxazine-6-carboxylic acid (1.0 g) (3), (±)-9-fluoro-2,3-dihydro-3-methyl-10-(1-piperazinyl)-7-oxo-7H-pyrido[1,2,3-de]-1,4-benzoxazine-6-carboxylic acid (0.92 g) (1), n-butanol (100 mL) and a few drops of cone HCl was refluxed with stirring for 96 hours. The solvent was removed under reduced pressure and the resulting sold was dissolved in a boiling mixture of methanol and water, decolorized with charcoal, filtered, cooled and the solid was removed by filtration to give 0.04 g of (5). m.p.>300° C.

Anal. Calcd. for: C₃₆H₃₉N₉F₂Cl₂O₈

| | C | H | N | F | Cl |
|---|---|---|---|---|---|
| Theory: | 51.80 | 4.70 | 15.10 | 4.55 | 8.49 |
| Found: | 51.04 | 5.47 | 11.03 | 4.17 | 8.56 |

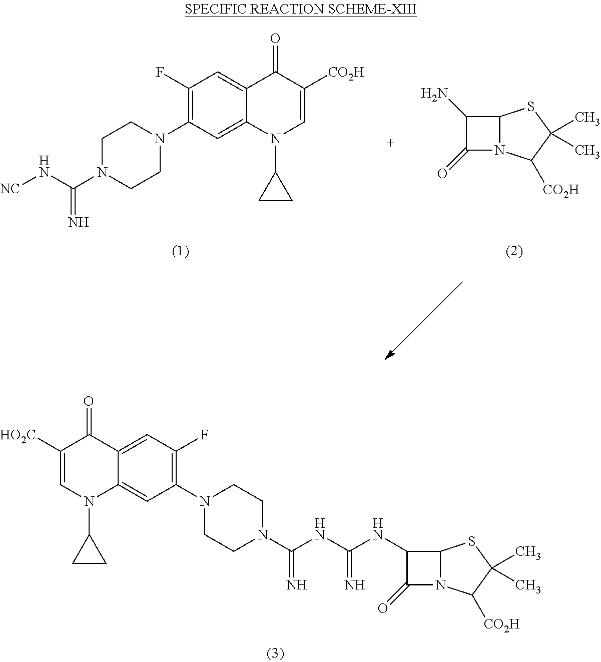 [SMILES: N#CNC(=N)N1CCN(c2cc3c(cc2F)c(=O)c(C(=O)O)cn3C2CC2)CC1 ; CC1(C)SC2C(N)C(=O)N2C1C(=O)O ; CC1(C)SC2C(NC(=N)NC(=N)N3CCN(c4cc5c(cc4F)c(=O)c(C(=O)O)cn5C4CC4)CC3)C(=O)N2C1C(=O)O]

### Method-1

A mixture of 1-cyclopropyl-6-fluoro-1,4-dihydro-4-oxo-7-(1-piperazinyl-4-cyanoguanidine)-3-quinolinecarboxylic acid (5 g) (1), 6-aminopenicilanic acid (2.7 g) (2), n-butanol (200 mL) and conc. HCl (1 mL) was refluxed with stirring for 24 hr. The solvent was removed under reduced pressure and the resulting solid was recrystallized twice from boiling methanol and decolorized with charcoal to yield 0.5 g of (3). Mp 290-293° C.

Anal. Calcd. for: C₂₇H₃₃N₃FCl₂SO₆

| | C | H | N |
|---|---|---|---|
| Theory: | 47.17 | 4.83 | 16.29 |
| Found: | 47.28 | 5.73 | 15.16 |

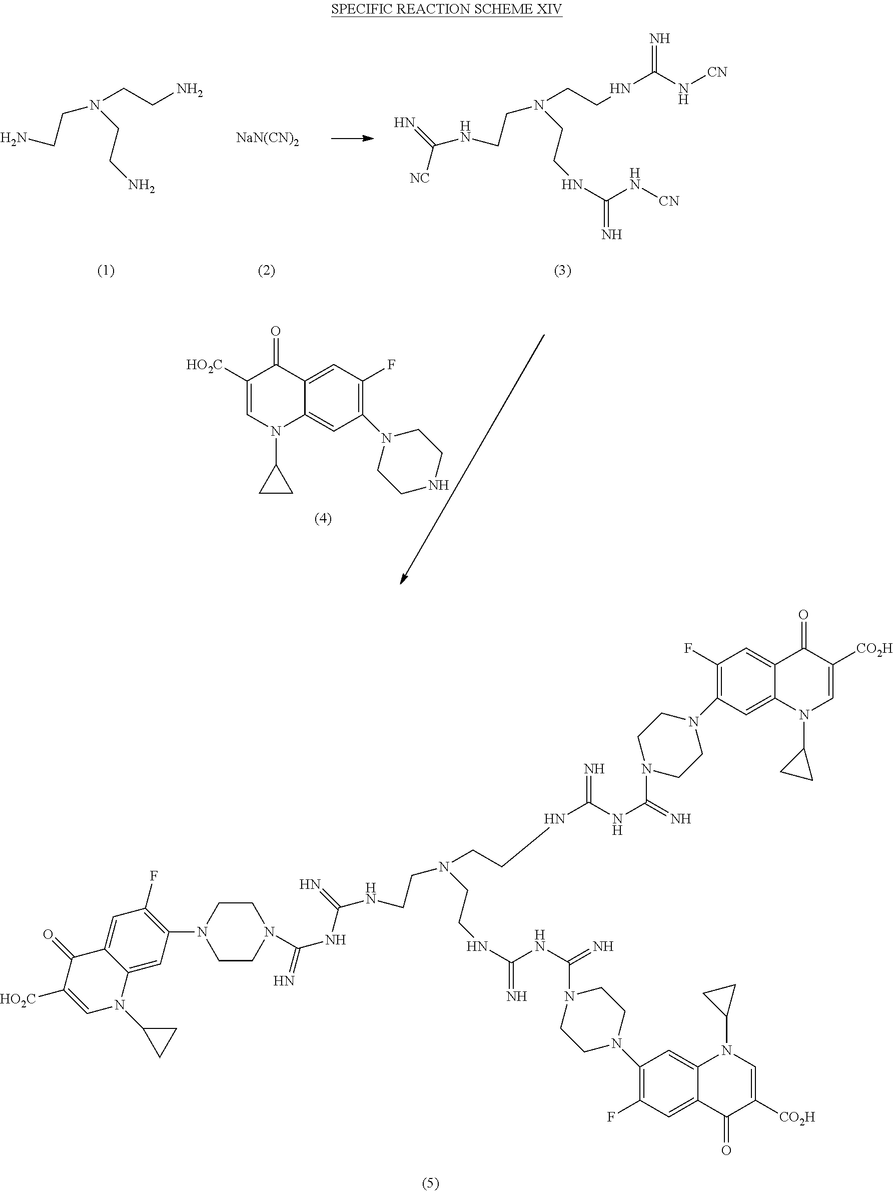

### Method-1

A mixture of Tris (2-aminoethyl)amine (10 g) (1) acidified with cone HCl and sodium dicyanamide (18.16 g) was heated at 195-200° C. for 0.5 hr. It was cooled, mixed with water, acidified with cone HCl, filtered and washed with water. The resulting sticky solid was mixed with water again, basified with 10% NaoH solution, filtered, and washed with water. The sticky mass was mixed and washed with ether repeatedly when it became solid. wt. 17.7 g (3) mp. 265-270° C.

Anal. Calcd. for: C₁₂H₂₁N₁₃

| | C | H |
|---|---|---|
| Theory: | 41.49 | 6.09 |
| Found: | 41.56 | 6.20 |

### Method-2

A thoroughly ground mixture of Tris (2-cyanoguanidinoamino ethyl) (1 g) (3), 1-cyclopropyl-6-fluoro-1,4-dihydro-4-oxo-7-(1-piperazinyl) (4) quinolinecarboxylic acid (3.16 g) (4) and a few drops of cone HCl was fused at 290-295° C. for 0.5 hr. It was cooled, dissolved in water, decolorized with activated charcoal, filtered, basified with 10% sodium carbonate solution and the precipitate was removed by filtration. The solid base was dissolved in water by acidifying with cone HCl, decolorized with charcoal again, filtered cooled and made turbid with acetone. The solid was removed by filtration to give 0.1 gm of (5). mp>300° C.

Anal. Calcd. for: C₆₃H₈₁N₂₂F₃Cl₆O₉

| | C | H | N | F | Cl |
|---|---|---|---|---|---|
| Theory: | 48.50 | 5.23 | 19.75 | 3.65 | 13.63 |
| Found: | 49.95 | 5.69 | 20.91 | 2.65 | 12.34 |

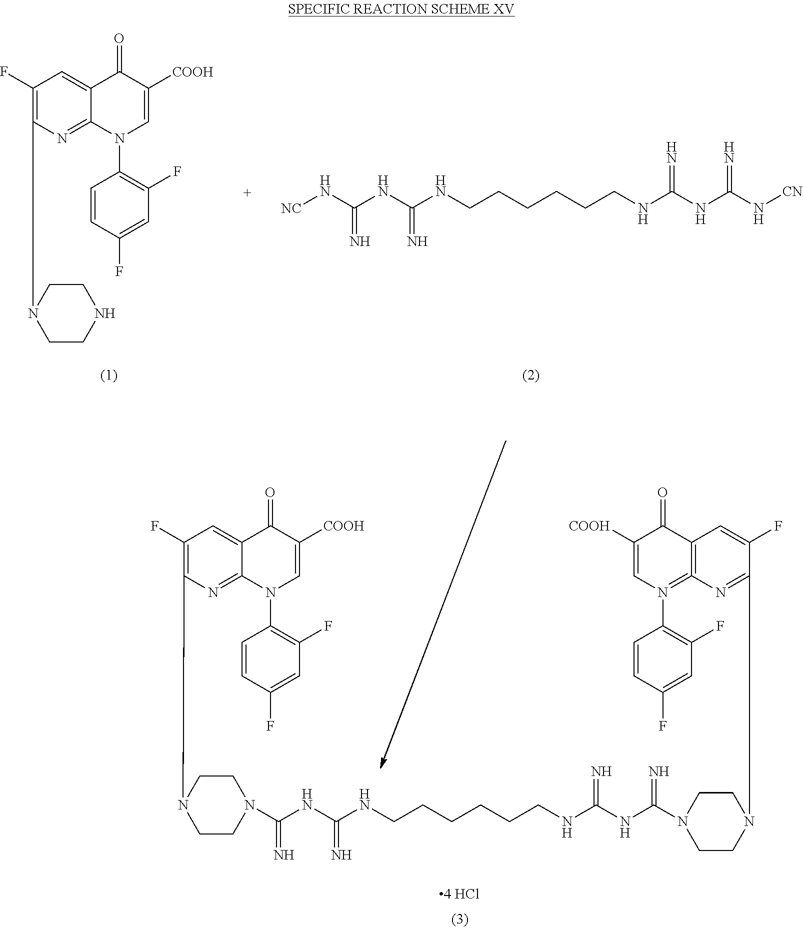

### Method-1

A mixture of (3.5 g.) (1), (1.08 g.) (2), n-butanol (150 mL), water (7 mL) and conc. HCL (2 mL) was refluxed with stirring for 106 hours, then evaporated to dryness. The resulting solid was washed with acetone and recrystallized twice from boiling methanol and decolorized with charcoal to yield 0.3 g. of (3).

Anal. Calcd. For: C₄₈H₅₂N₁₆F₆Cl₄O₆

| | C | H | N | Cl |
|---|---|---|---|---|
| Theory: | 47.85 | 4.34 | 18.60 | 11.77 |
| Found: | 47.92 | 4.65 | 16.7 | 10.07 |

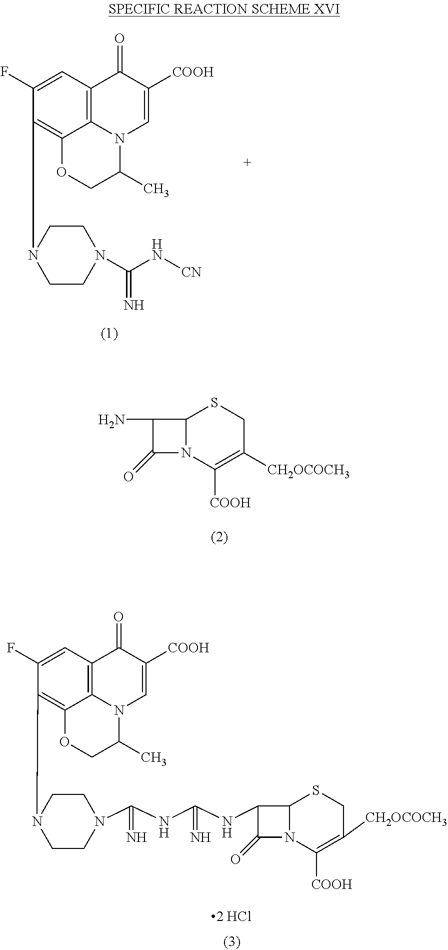

### Method-1

A mixture of (±)-9-Fluoro-2,3-dihydro-3-methyl-10(4-cyanoguanidino-1-piperazinyl)-7-oxo-7H-pyrido 1,2,3-de-1,4-benzoxazine-6-carboxylic acid (1.5 g)(1), 7-amino cephalosporanic acid (1 g.) (2), n-butanol (100 mL) and conc. HCl (0.5 mL) was refluxed with stirring for 29 hr. It was cooled on ice and the solid was removed by filtration. It was washed with acetone and the resulting solid was recrystallized twice from boiling methanol and decolorized with charcoal to give 0.08 g. of (3).

Anal. Calcd. for: C₂₉H₃₃N₈FCl₂SO₉

| | C | H | N | F |
|---|---|---|---|---|
| Theory: | 45.55 | 4.35 | 14.65 | 2.48 |
| Found: | 46.84 | 5.26 | 14.62 | 3.87 |

Tables I-IV below list additional preferred cyclam and bicyclam derivatives within the scope of the present invention. In the tables, the “structure” column refers to the numbered structures depicted following the tables.

TABLE I

| Struc- | | | | |
|---|---|---|---|---|
| ture | n | X | Y | R |
| 1, 2, 3, 4, 5, 6, 7, 8, 14 9, 10, 12, 15 11, 13, 16 | 1-6 | N, CH, C—CH₃, C—OCH₃, C—F C—OCHF₂ C—OCH₂F C—OCHCl₂ C—OCH₂Cl | H, Br, Cl F, NO₂ Alkyl | 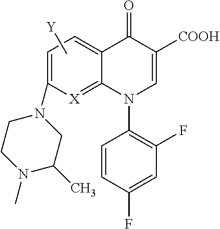 |
| 1, 2, 3, 4, 5, 6, 7, 8, 14 9, 10, 12, 15 11, 13, 16 | 1-6 | N, CH, C—CH₃, C—OCH₃, C—F C—OCHF₂ C—OCH₂F C—OCHCl₂ C—OCH₂Cl | H, Br, Cl F, NO₂ Alkyl | 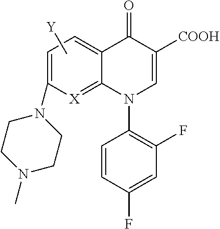 |
| 1, 2, 3, 4, 5, 6, 7, 8, 14 9, 10, 12, 15 11, 13, 16 | 1-6 | N, CH, C—CH₃, C—OCH₃, C—F C—OCHF₂ C—OCH₂F C—OCHCl₂ C—OCH₂Cl | H, Br, Cl F, NO₂ Alkyl | 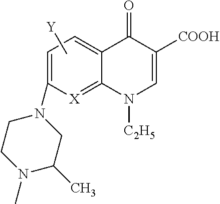 |
| 1, 2, 3, 4, 5, 6, 7, 8, 14 9, 10, 12, 15 11, 13, 16 | 1-6 | C—OCHF₂ C—OCH₂F C—OCHCl₂ C—OCH₂Cl | H, Br, Cl F, NO₂ Alkyl | 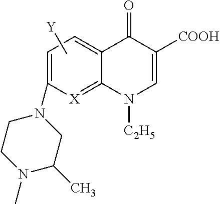 |

TABLE II

| Structure | n | X | Y | R |
|---|---|---|---|---|
| 1, 2, 3, 4, 5, 6, 7, 8, 14 9, 10, 12, 15 11, 13, 16 | 1-6 | N, CH, C—CH₃, C—OCH₃, C—F C—OCHF₂ C—OCH₂F C—OCHCl₂ C—OCH₂Cl | H, Br, Cl, F, NO₂ Alkyl | 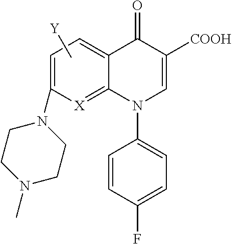 |
| 1, 2, 3, 4, 5, 6, 7, 8, 14 9, 10, 12, 15 11, 13, 16 | 1-6 | N, CH, C—CH₃, C—OCH₃, C—F C—OCHF₂ C—OCH₂F C—OCHCl₂ C—OCH₂Cl | H, Br, Cl, F, NO₂ Alkyl | 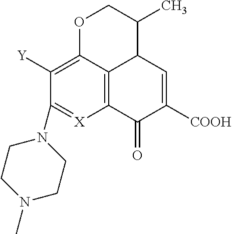 |
| 1, 2, 3, 4, 5, 6, 7, 8, 14 9, 10, 12, 15 11, 13, 16 | 1-6 | N, CH, C—CH₃, C—OCH₃, C—F C—OCHF₂ C—OCH₂F C—OCHCl₂ C—OCH₂Cl | H, Br, Cl, F, NO₂ Alkyl | 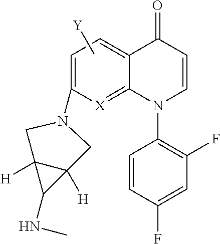 |

TABLE III

| Structure | n | X | Y | R |
|---|---|---|---|---|
| 1, 2, 3, 4, 5, 6, 7, 8, 14 9, 10, 12, 15 11, 13, 16 | 1-6 | N, CH, C—CH₃, C—OCH₃, C—F C—OCHF₂ C—OCH₂F C—OCHCl₂ C—OCH₂Cl | H, Br, Cl F, NO₂ Alkyl | 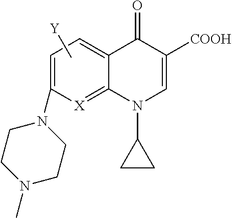 |
| 1, 2, 3, 4, 5, 6, 7, 8, 14 9, 10, 12, 15 11, 13, 16 | 1-6 | | Br, Cl F, NO₂ Alkyl | 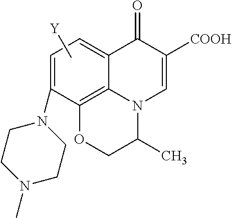 |
| 1, 2, 3, 4, 5, 6, 7, 8, 9, 10,12, 15 11, 13, 16 | 1-6 | N, CH, C—CH₃, C—OCH₃, C—F C—OCHF₂ C—OCH₂F C—OCHCl₂ C—OCH₂Cl | Br, Cl F, NO₂ Alkyl | 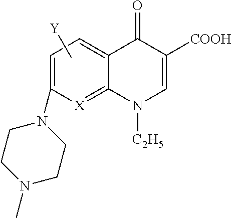 |
| 1, 2, 3, 4, 5, 6, 7, 8, 14 9, 10, 12, 15 11, 13, 16 | 1-6 | C—OCHF₂ C—OCH₂F C—OCHCl₂ C—OCH₂Cl | Br, Cl F, NO₂ Alkyl | 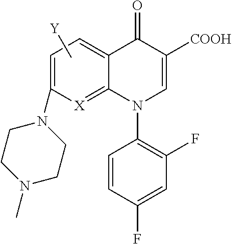 |

TABLE IV

| Structure | n | X | Y | R |
|---|---|---|---|---|
| 1, 2, 3, 4, 5, 6, 7, 8, 14 9, 10, 12, 15 11, 13, 16 | 1-6 | N, CH, C—CH₃, C—OCH₃, C—F C—OCHF₂ C—OCH₂F C—OCHCl₂ C—OCH₂Cl | H, I, Br, Cl F, NO₂ Alkyl | 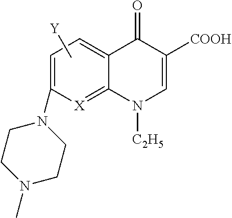 |
| 1, 2, 3, 4, 5, 6, 7, 8, 14 9, 10, 12, 15 11, 13, 16 | 1-6 | N, CH, C—CH₃, C—OCH₃, C—F C—OCHF₂ C—OCH₂F C—OCHCl₂ C—OCH₂Cl | Br, Cl, F, NO₂ Alkyl I | 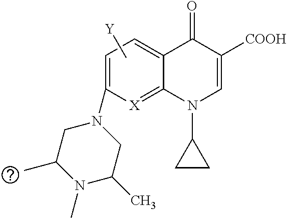 |
| | | N, CH, C—CH₃, C—OCH₃, C—F C—OCHF₂ C—OCH₂F C—OCHCl₂ C—OCH₂Cl | Br, Cl, F, NO₂, I, Alkyl | 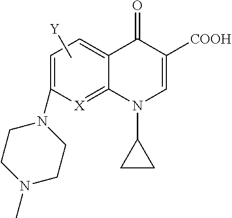 |
| | | C—OCHF₂ C—OCH₂F C—OCHCl₂ C—OCH₂Cl | Br, Cl F, NO₂, I, Alkyl | 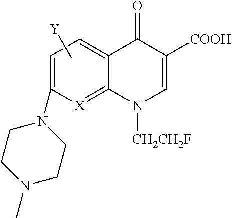 |

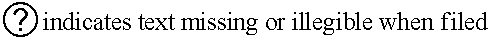

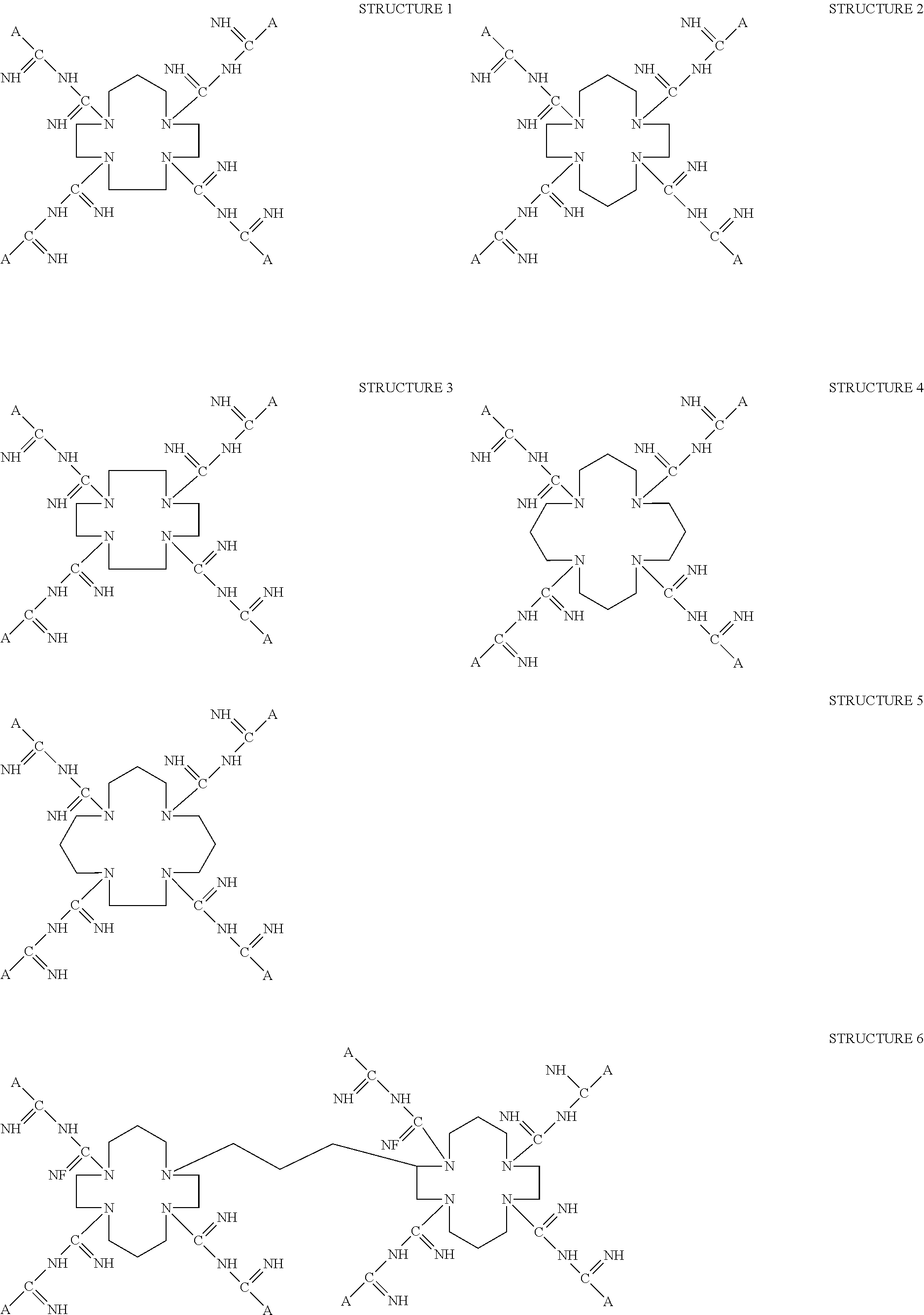

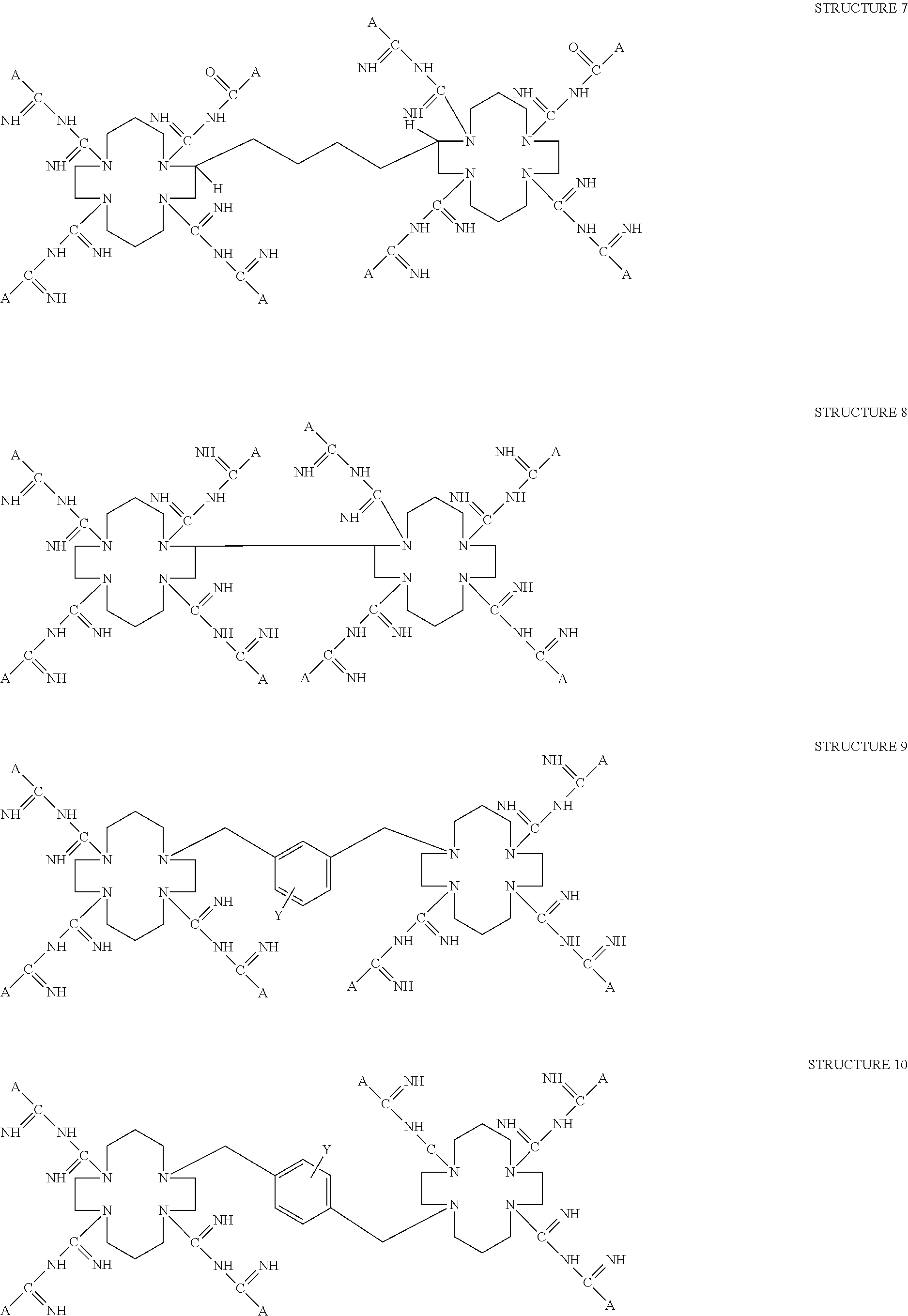

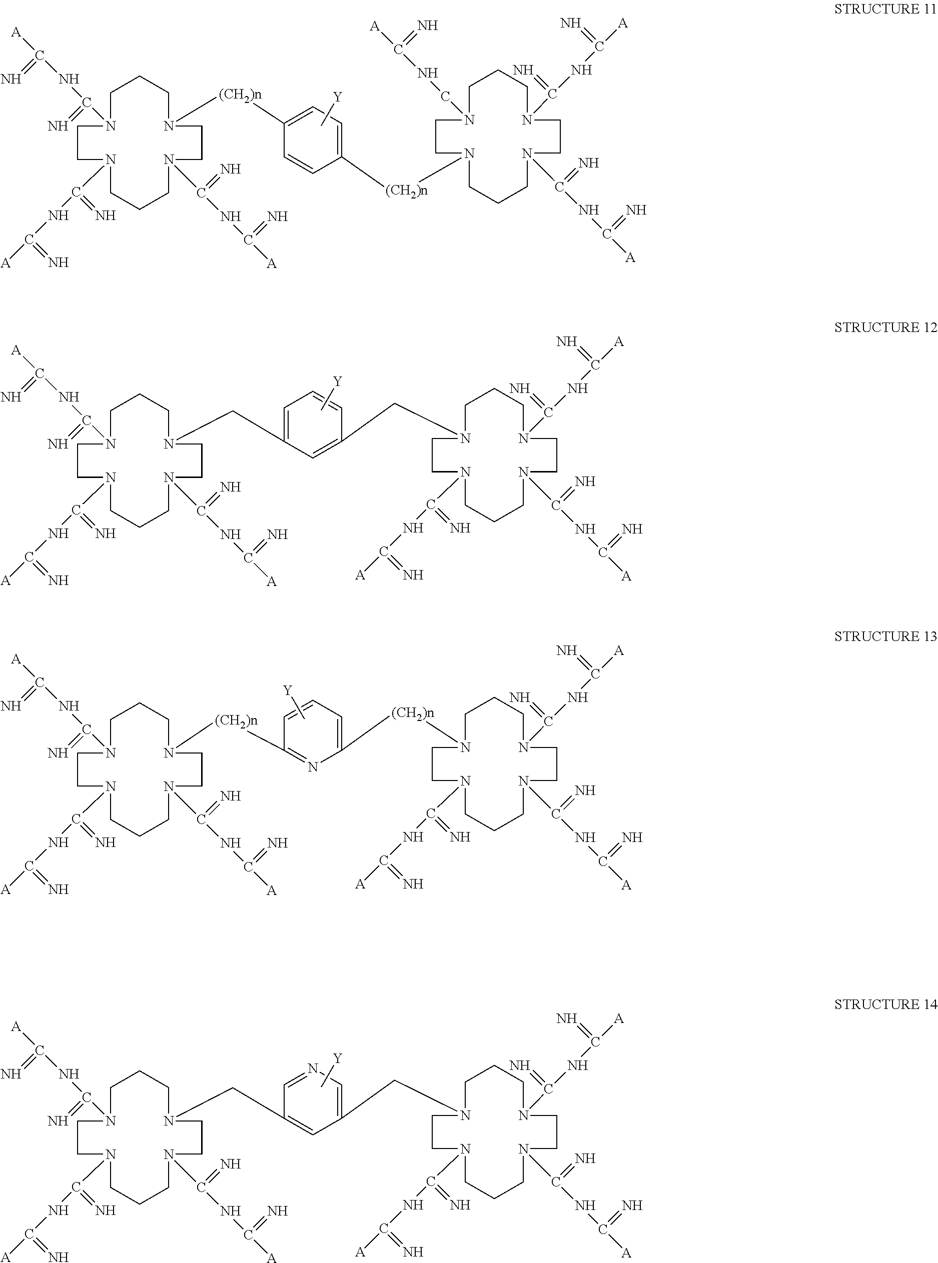

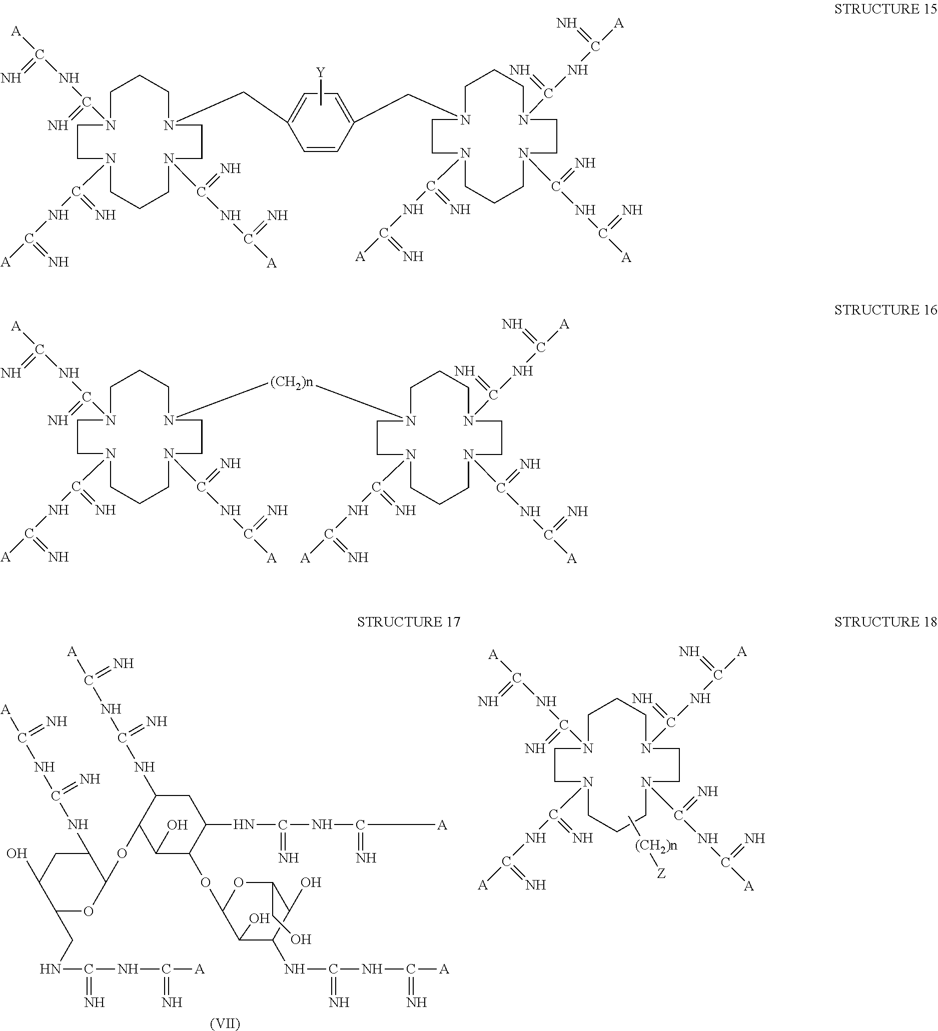

Compounds of the general structure (VIII) depicted above may be synthesized, for example, by reacting a suitable cyclam starting material with sodium dicyandiamide to form an intermediate, then reacting the intermediate with the desired antibacterial group(s) to form the final product. The reaction scheme below is an example of such a synthesis:

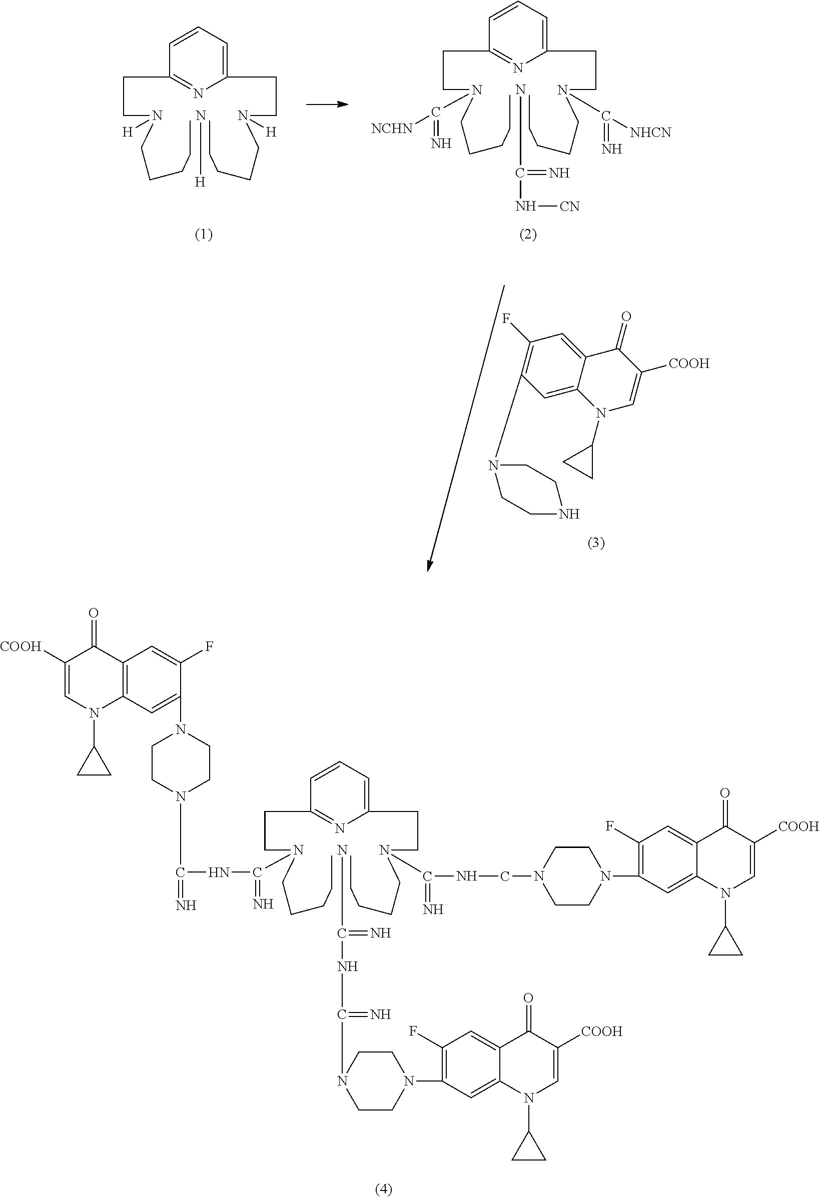

Compounds of the general structure (IX) depicted above may be synthesized in similar fashion using the appropriate bicyclam starting material. An example is depicted below.

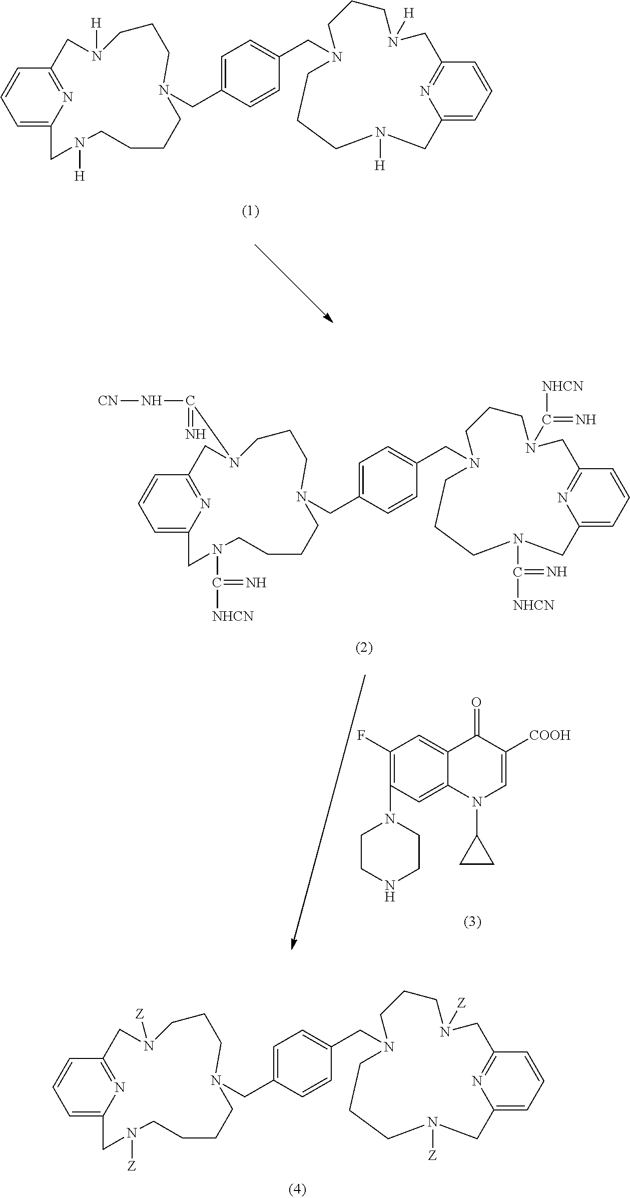

where each Z is

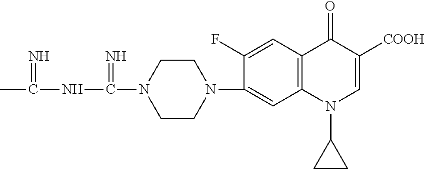 [SMILES: CC(=N)NC(=N)N1CCN(c2cc3c(cc2F)c(=O)c(C(=O)O)cn3C2CC2)CC1]

Compounds of the general structure (VII) depicted above may be synthesized in similar fashion using, for example, tobramycin as a starting material, which may be reacted with sodium dicyandiamide to give an intermediate product, which may then be reacted with the desired “A” group to form the final product.

Other specific compounds according to the present invention include:

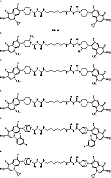

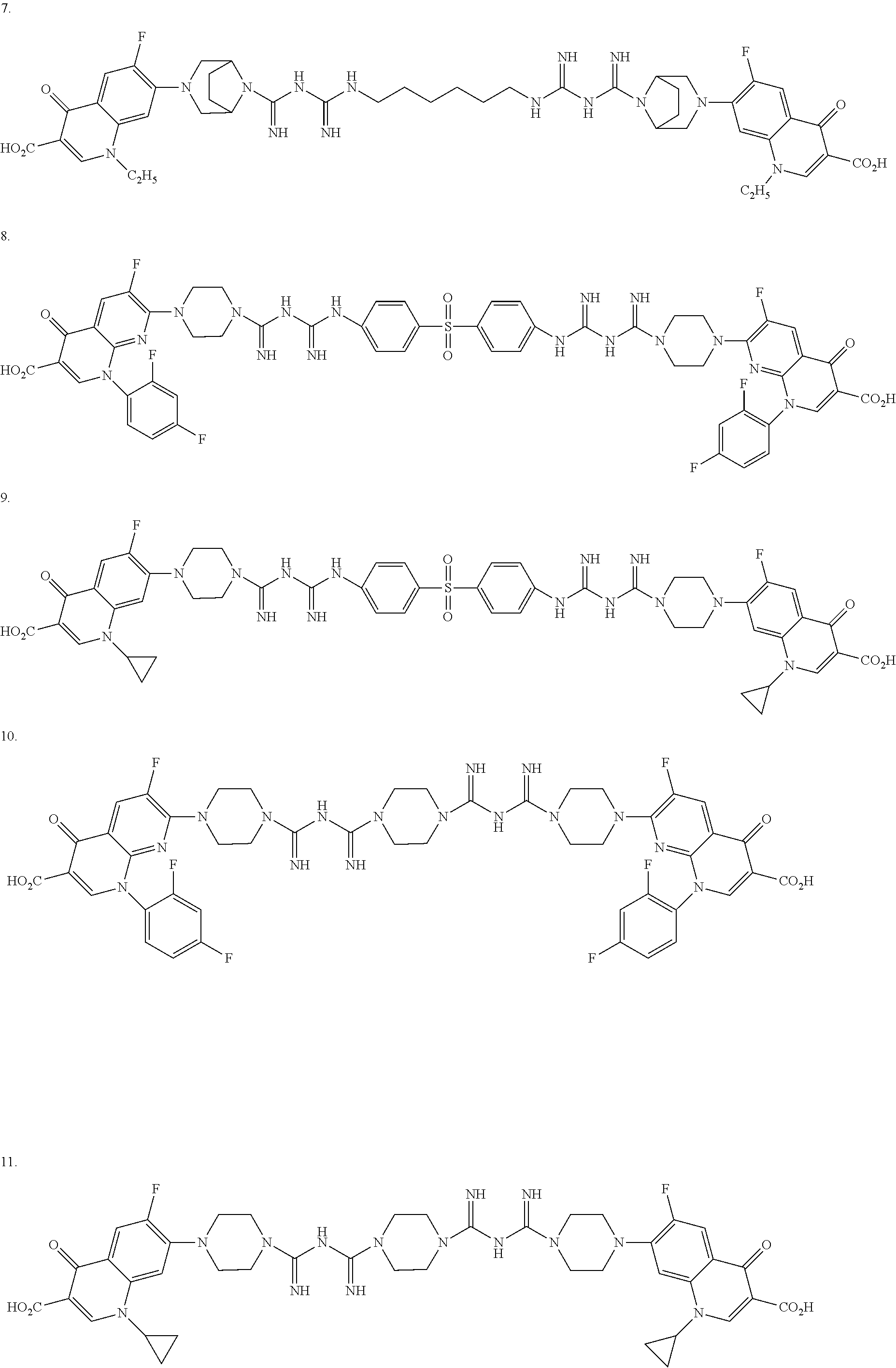

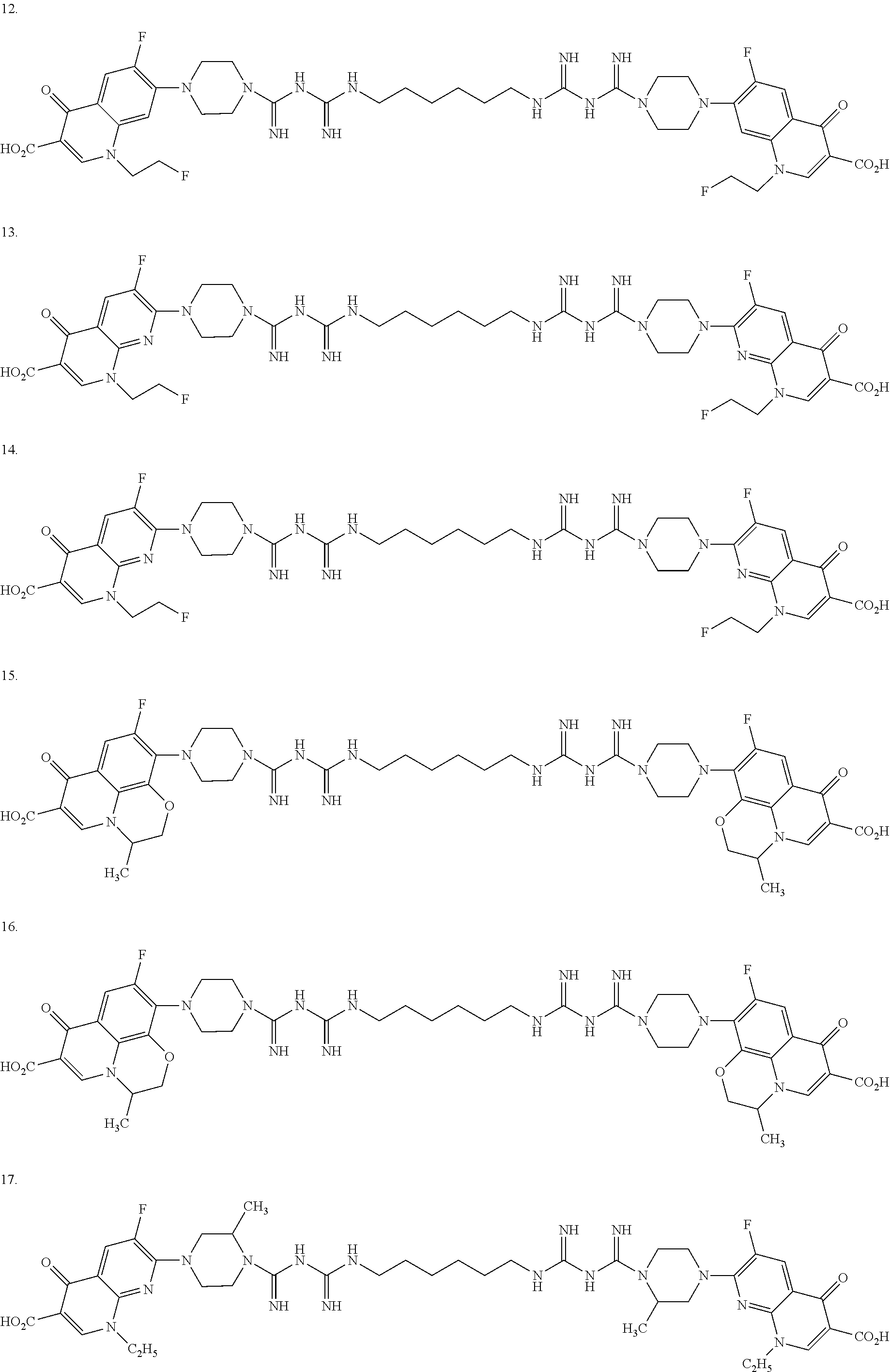

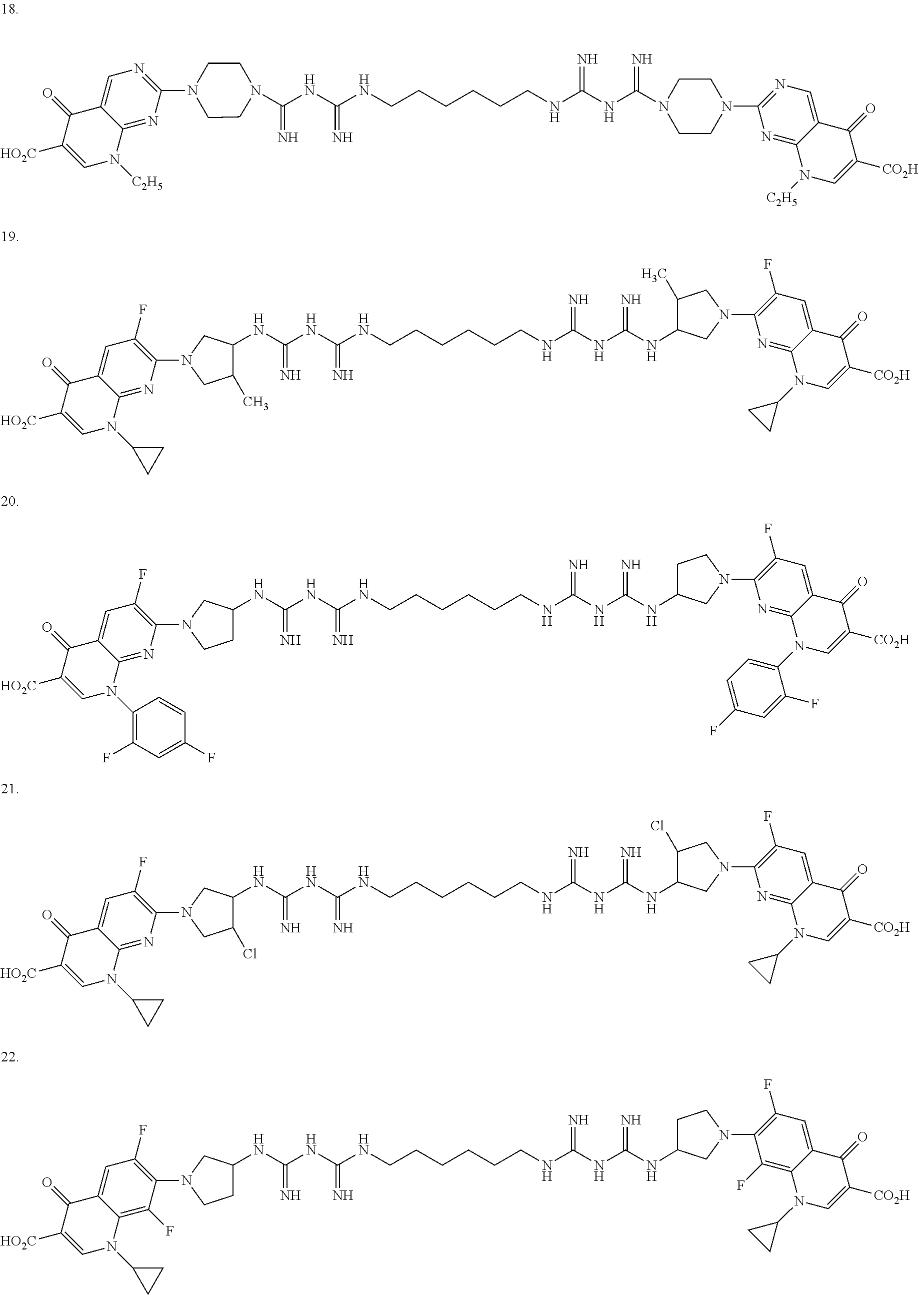

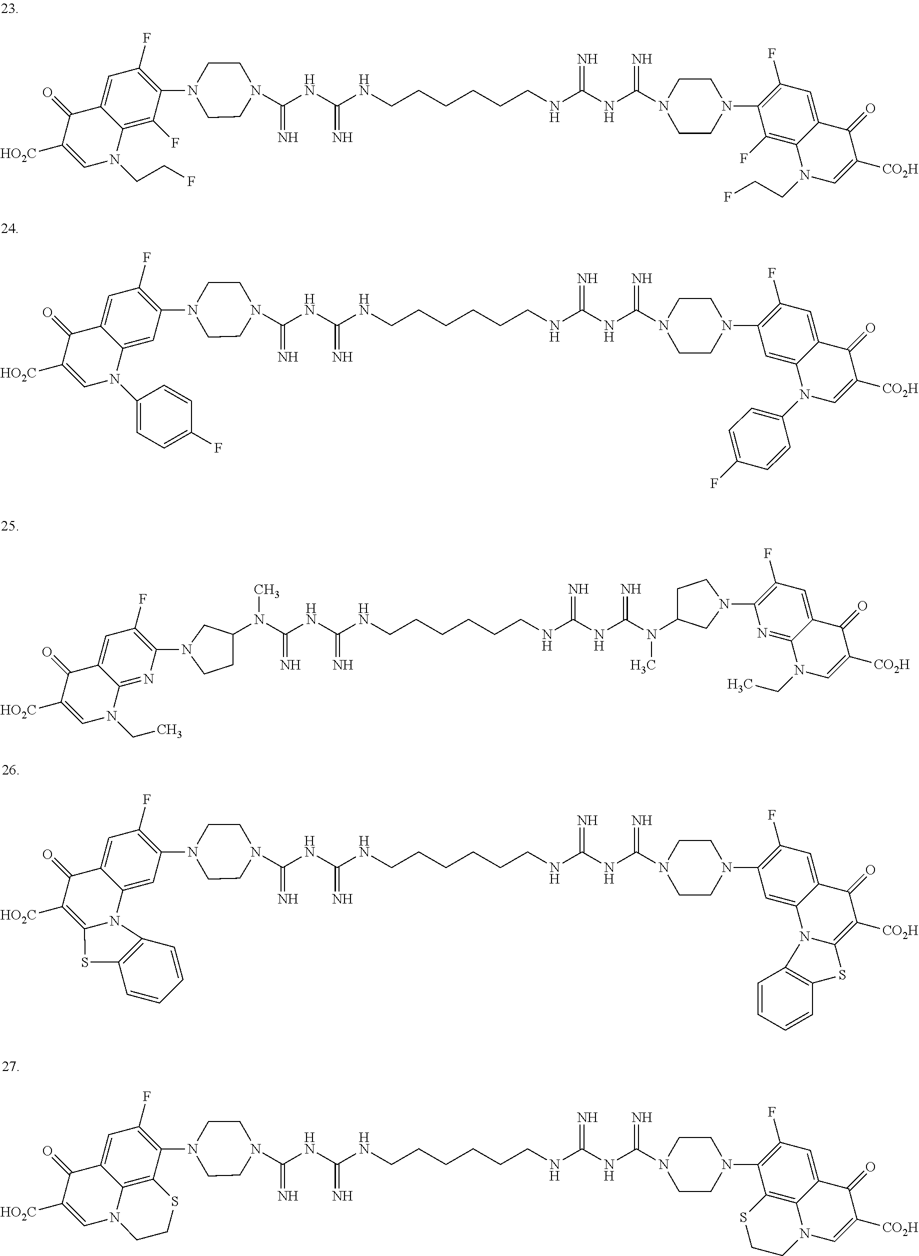

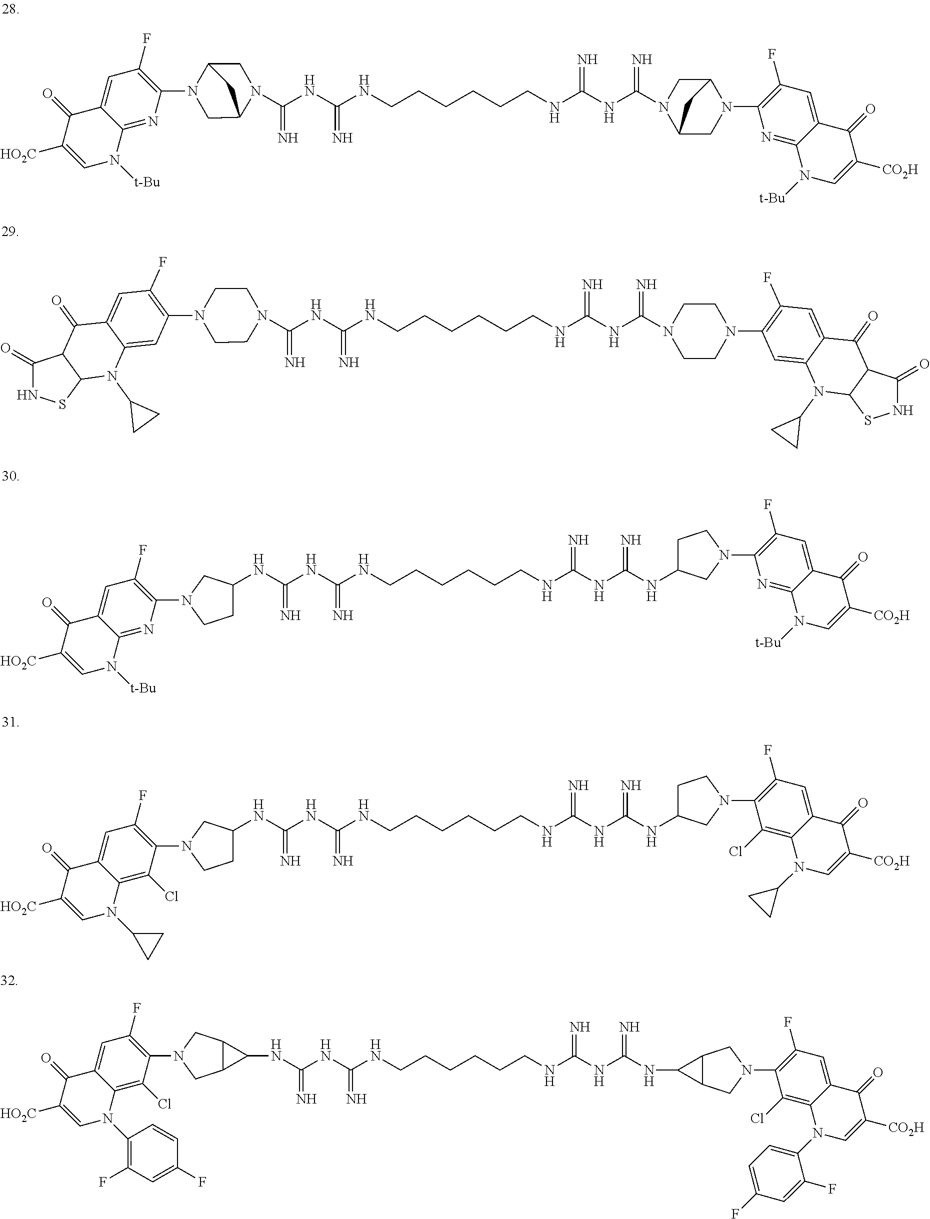

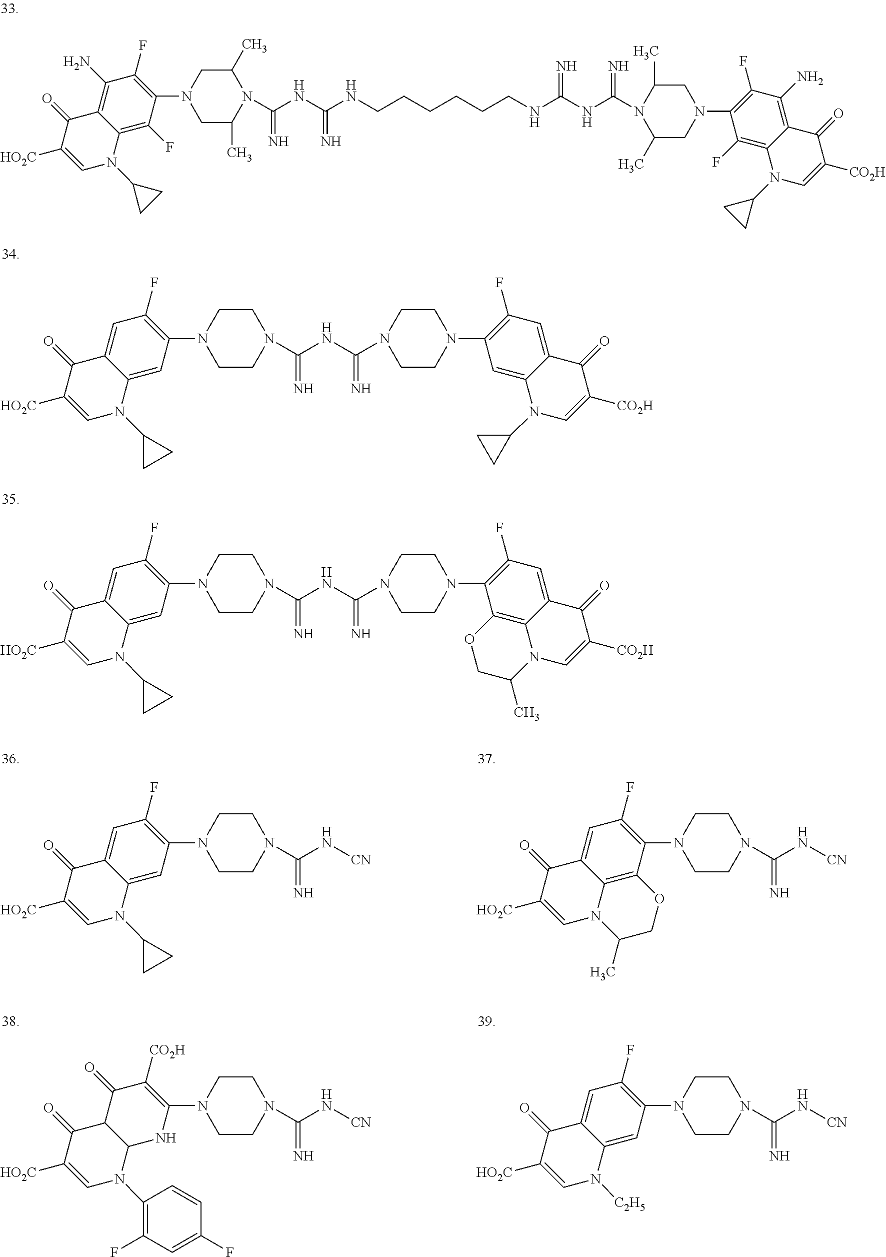

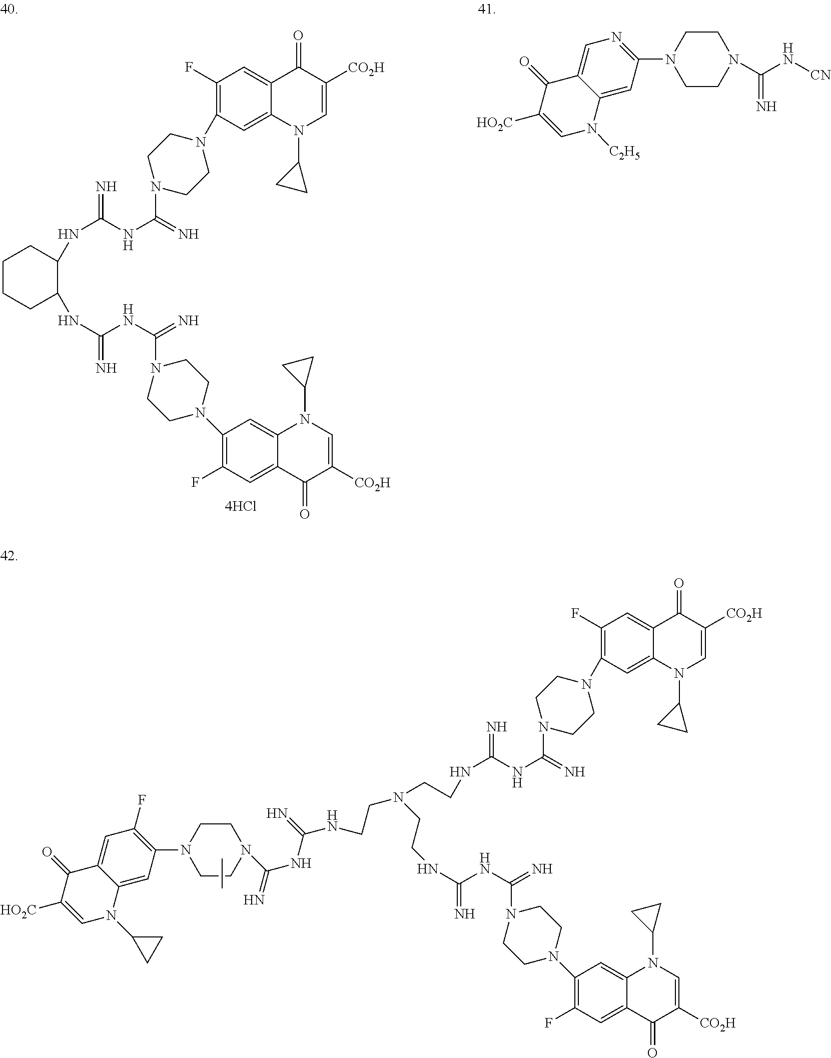

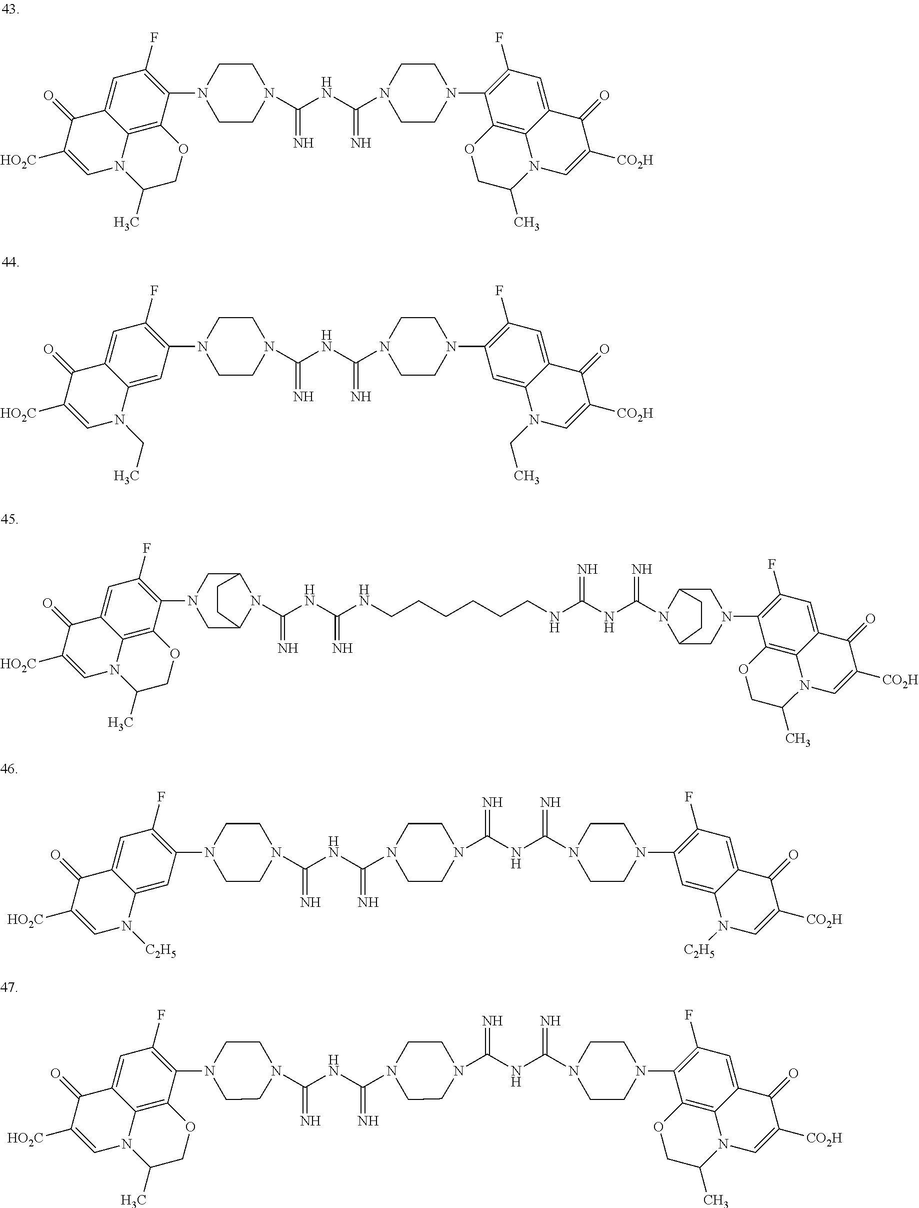

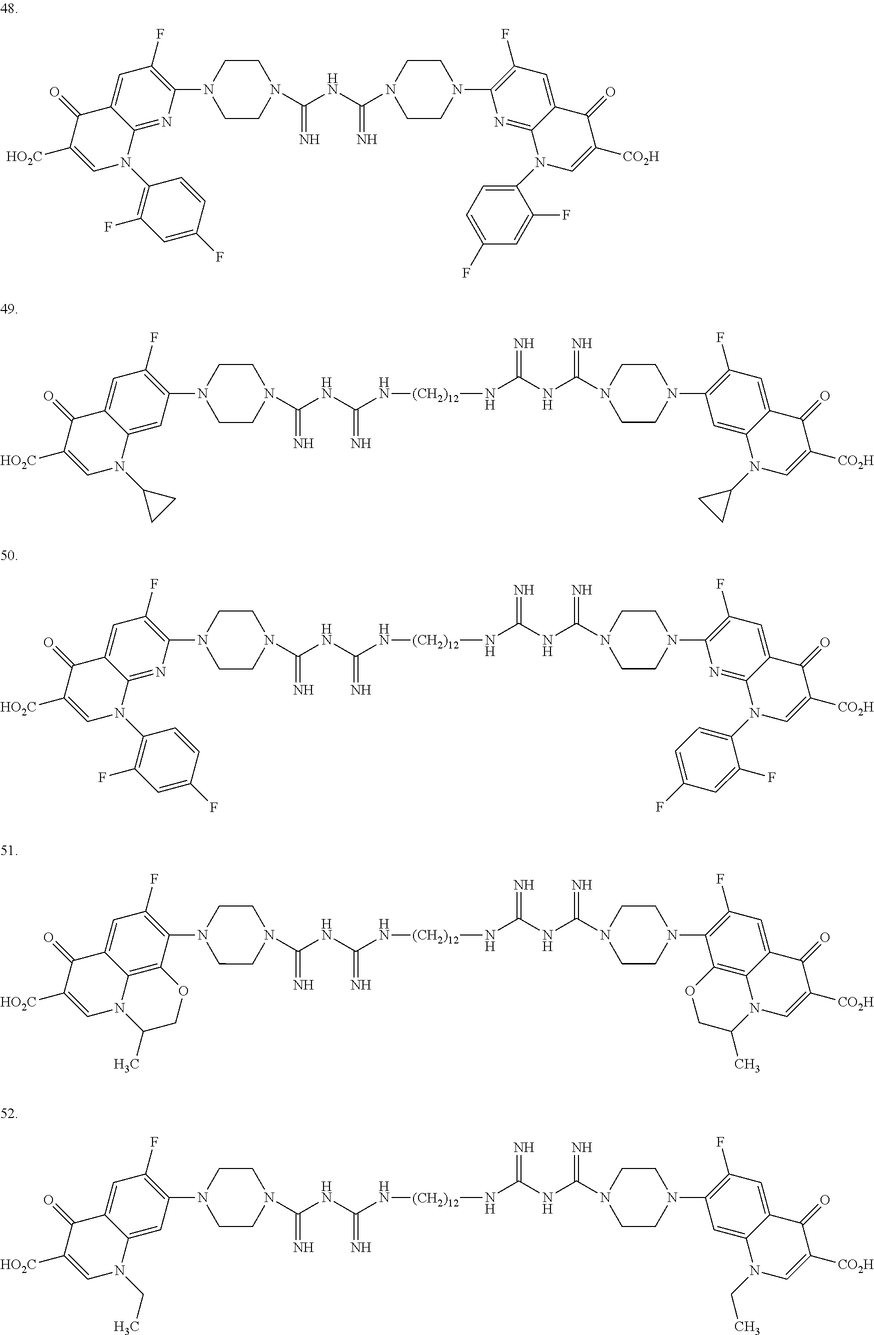

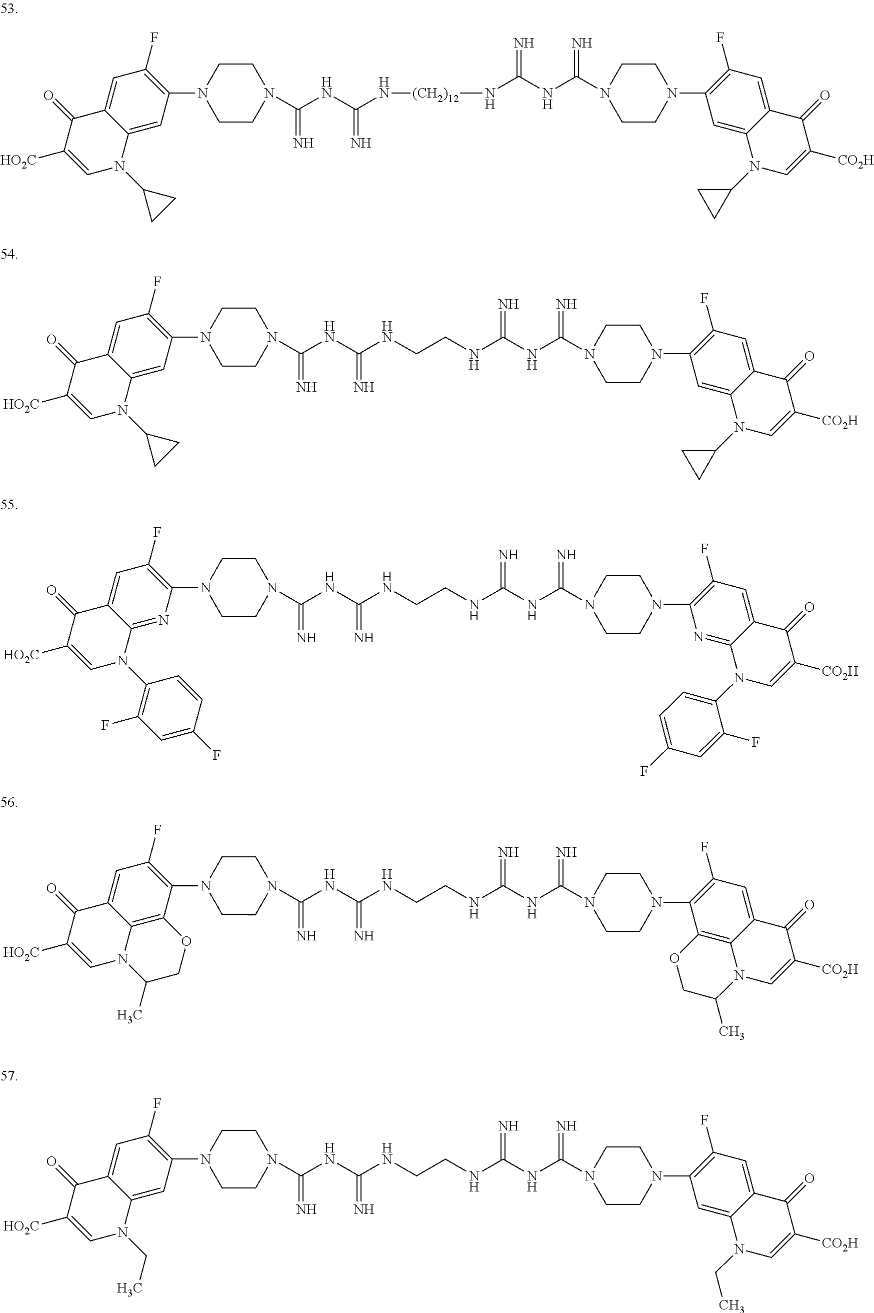

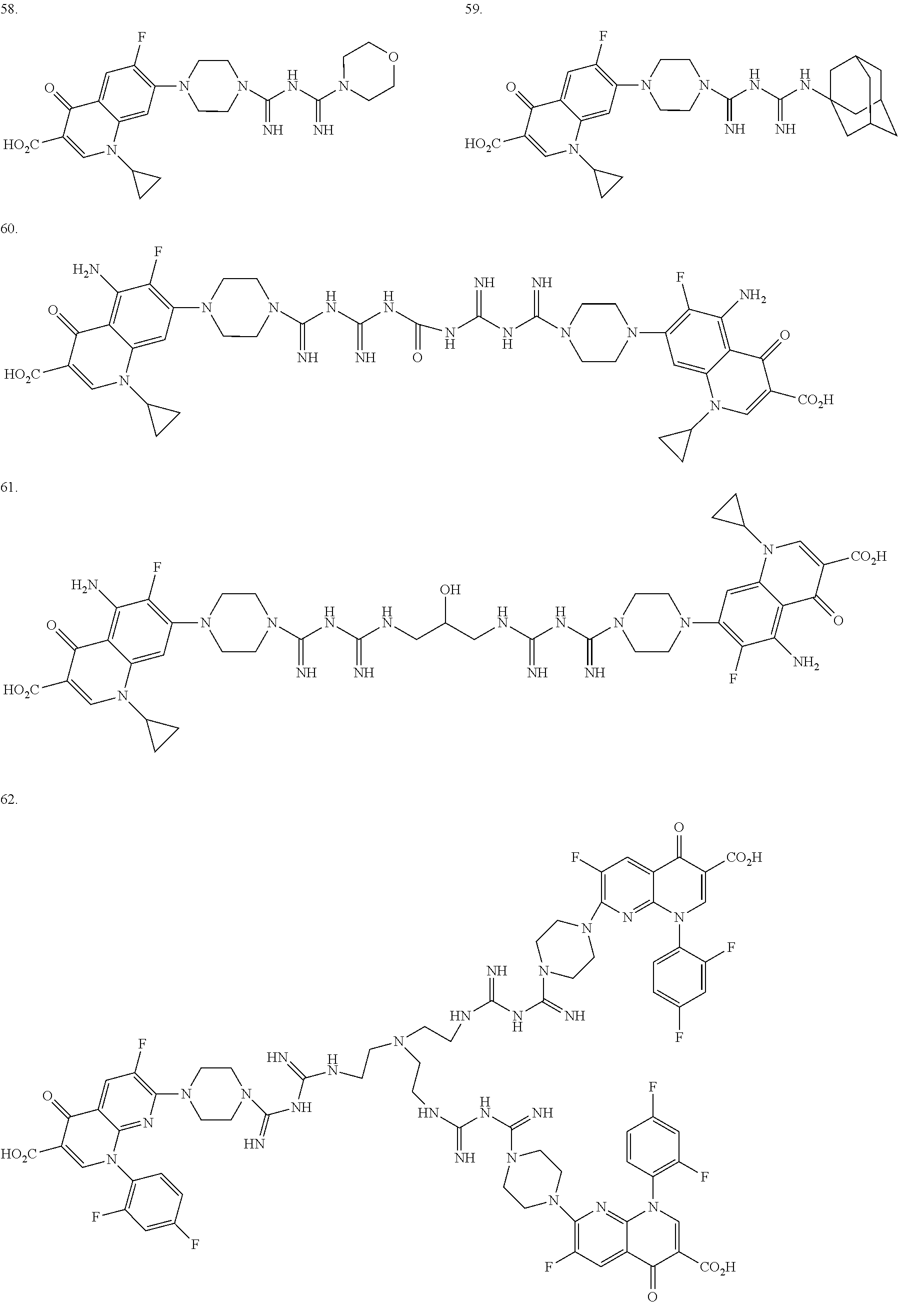

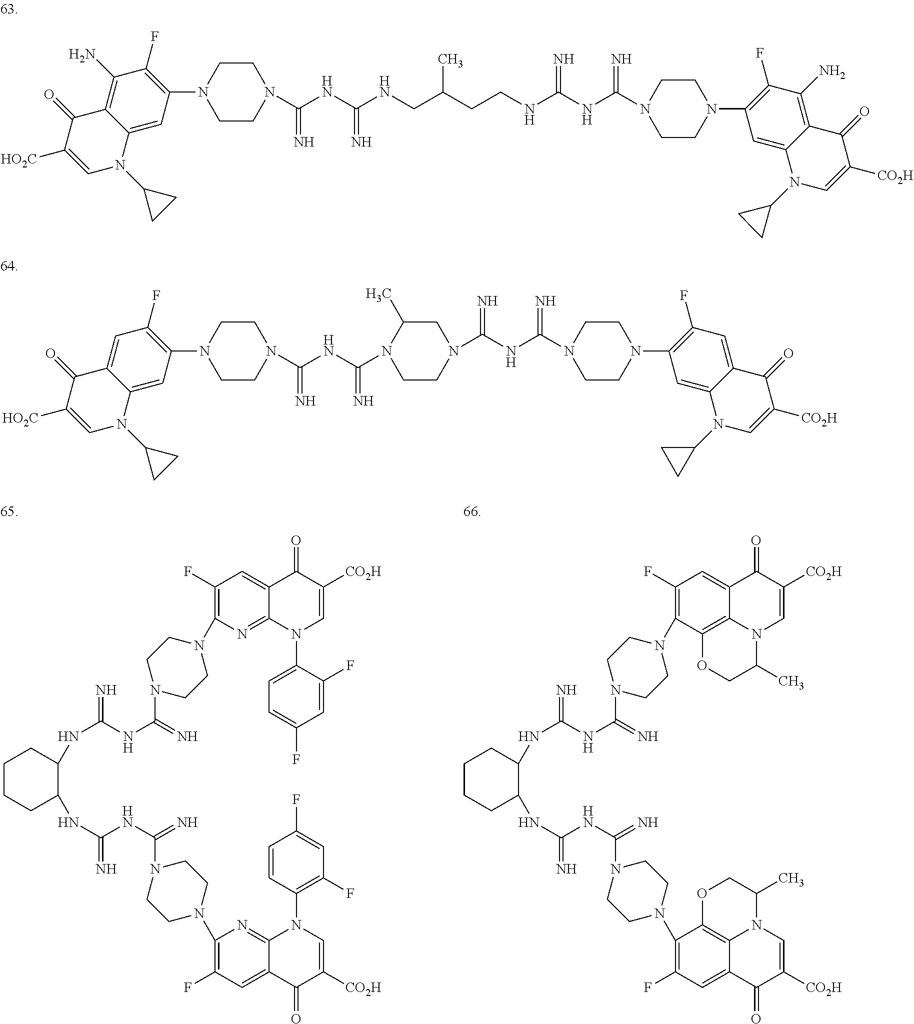

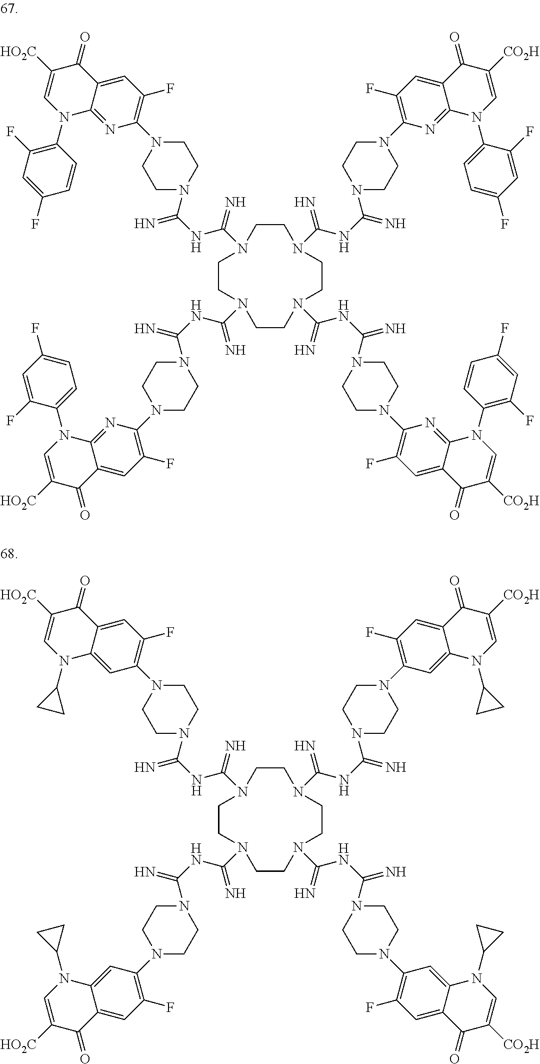

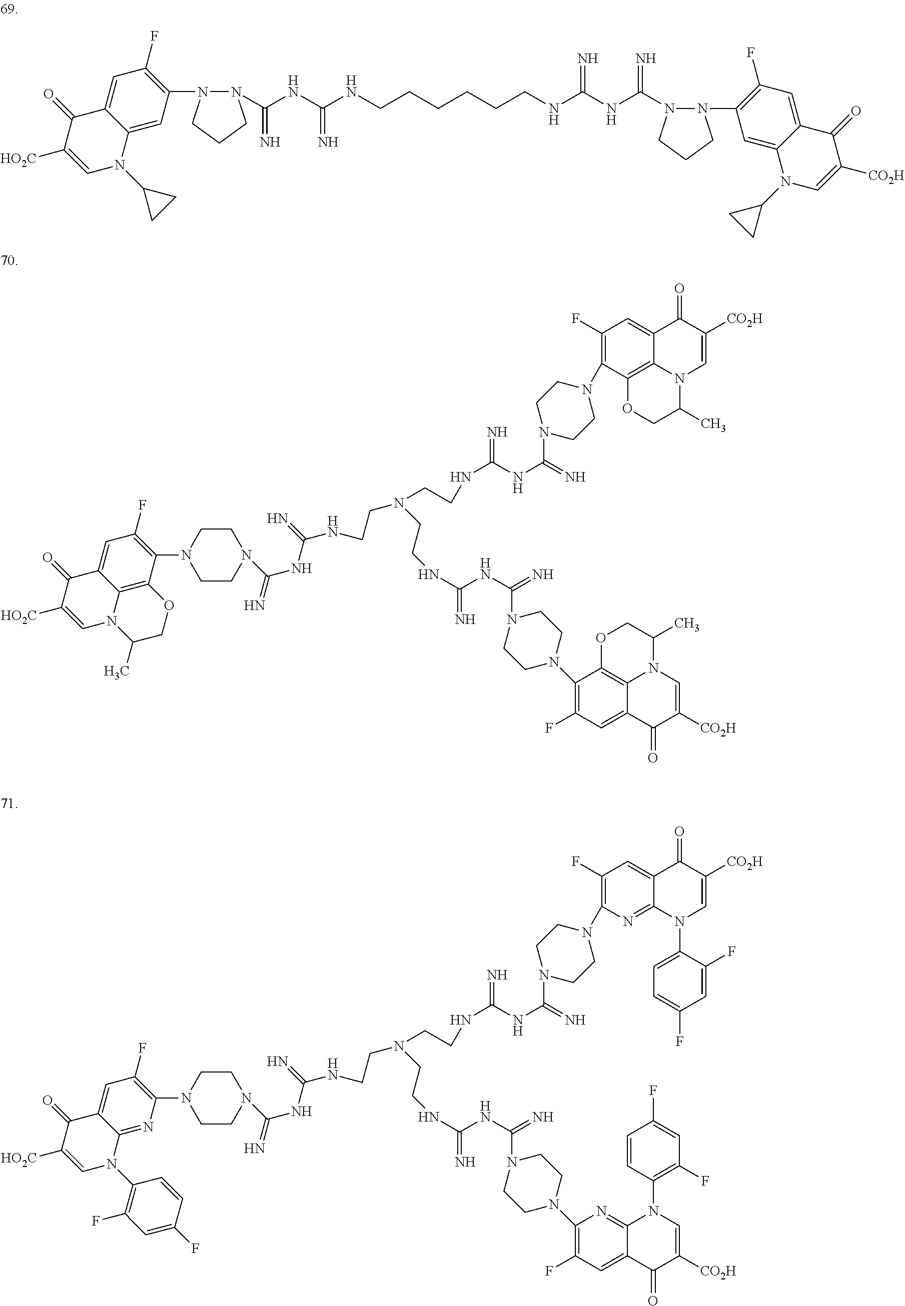

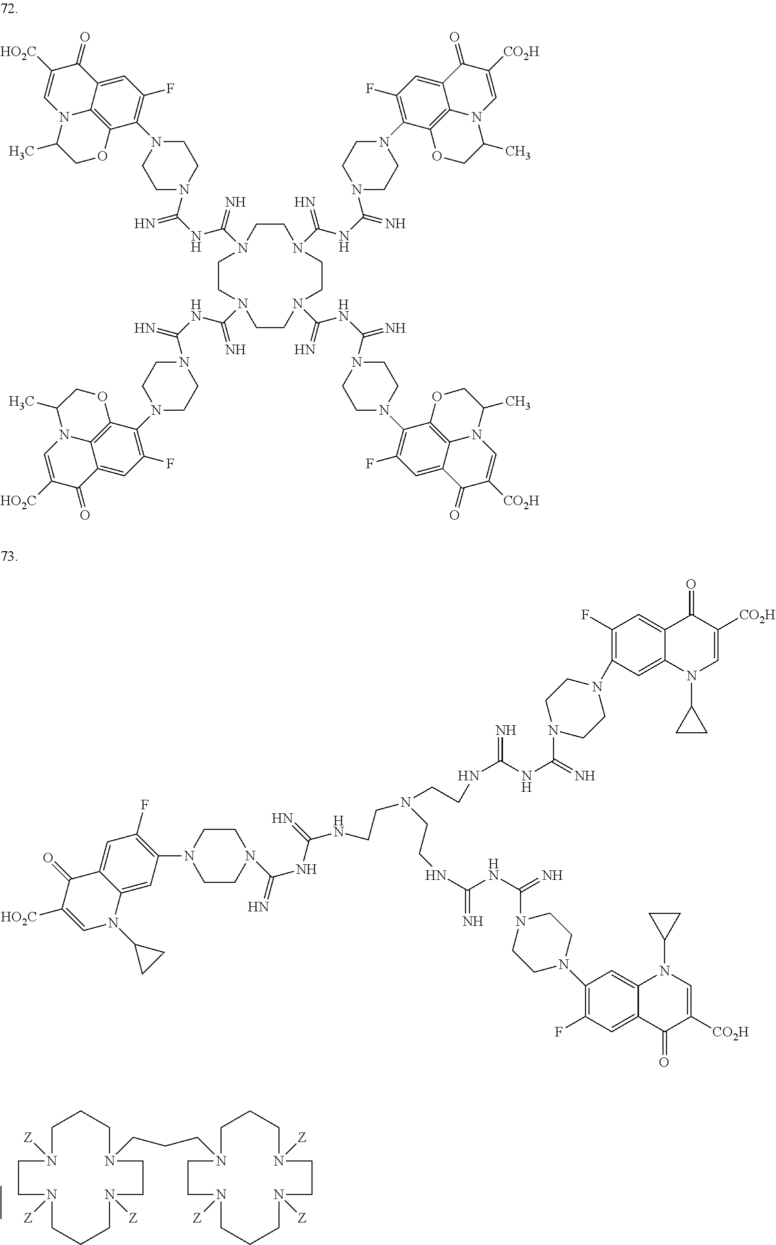

where each Z is:

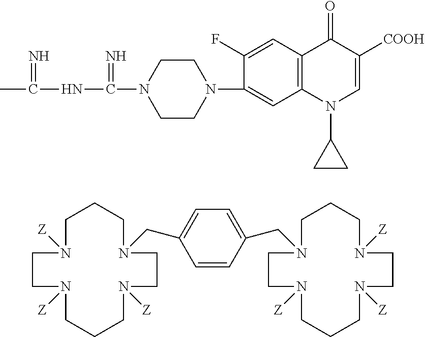

where each Z is:

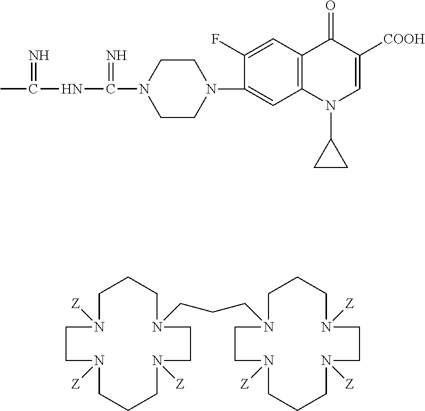

where each Z is:

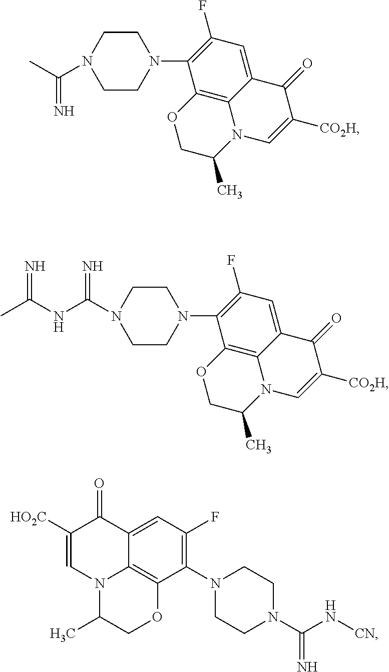 [SMILES: CC(=N)N1CCN(c2c(F)cc3c(=O)c(C(=O)O)cn4c3c2OC[C@@H]4C)CC1 ; CC(=N)NC(=N)N1CCN(c2c(F)cc3c(=O)c(C(=O)O)cn4c3c2OC[C@@H]4C)CC1 ; CC1COc2c(N3CCN(C(=N)NC#N)CC3)c(F)cc3c(=O)c(C(=O)O)cn1c23]

or a combination thereof.

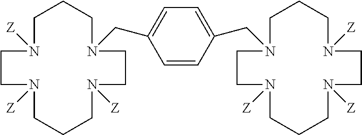

where each Z is:

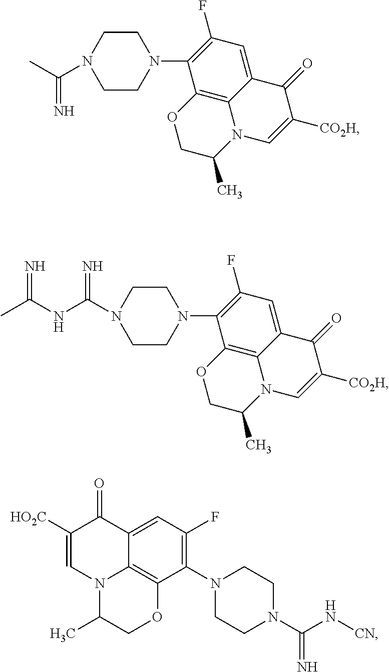 [SMILES: CC(=N)N1CCN(c2c(F)cc3c(=O)c(C(=O)O)cn4c3c2OC[C@@H]4C)CC1 ; CC(=N)NC(=N)N1CCN(c2c(F)cc3c(=O)c(C(=O)O)cn4c3c2OC[C@@H]4C)CC1 ; CC1COc2c(N3CCN(C(=N)NC#N)CC3)c(F)cc3c(=O)c(C(=O)O)cn1c23]

or a combination thereof.

What is claimed is:

1. A compound having one of the following structures: 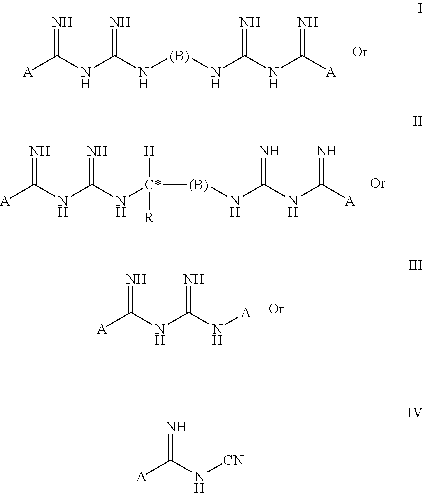
wherein:
a) each A is the same or different, and is selected from the group consisting of i) hydrogen, ii) a nitrile, iii) an amino, iv) an antibacterial agent, v) an antibiotic, vi) a quinolone, vii) an azaquinolone, and viii) one of the following groups: 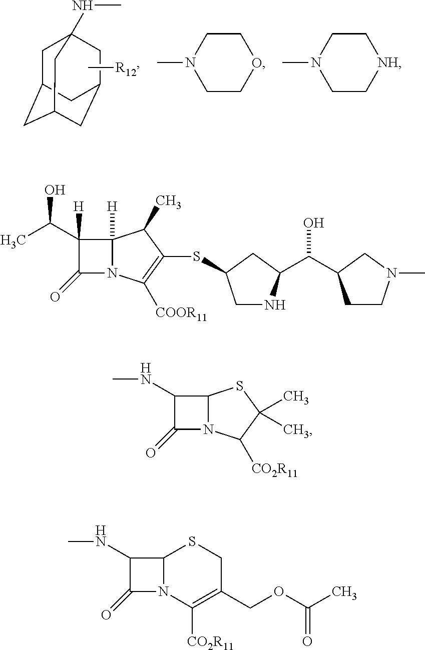
where R₁₂ is hydrogen or C₁-C₆ straight or branched alkyl, and R₁₁ is hydrogen, lower alkyl, an aromatic group or a heterocyclic group, with the proviso that with respect to structures I-III, both A's cannot be the adamantane structure above at the same time;
b) B is a straight chain or branched C₁-C₃₀ alkyl group, which may be interrupted by oxygen, sulfur, optionally substituted aromatic nuclei, sulphoxide, optionally substituted cyclohexane, nitrogen optionally substituted with —NH—C(NH)—NH—C(NH)-A where A is defined above, tris (2-aminoethyl)amine, a heterocycle of the following structure: 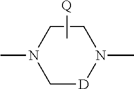
where D is 1-3 carbon atoms and Q is hydrogen, halogen or lower alkyl; or 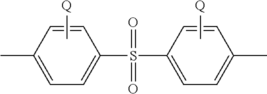
where Q is hydrogen, halogen or lower alkyl; or 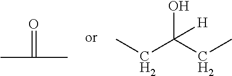
a hydrophilic moiety; and
c) R is hydrogen or C₁-C₆ straight or branched alkyl.

2. The compound of claim 1, wherein A is an antibacterial agent selected from the group consisting of 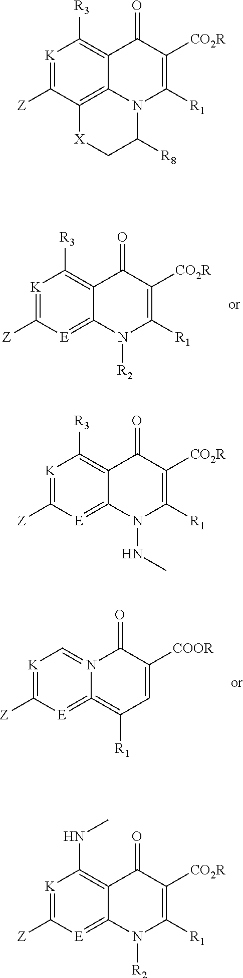
where Z is one or more heterocyclic rings containing at least one N atom
or 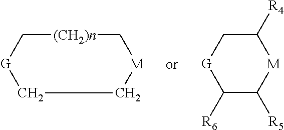
which may be attached to the structure above through any available point of attachment;
where n is 0 to 3; R₄, R₅ and R₆ are each independently hydrogen or lower alkyl or alkylene; G and M are independently O, S or NR₁₀; where R₁₀ is hydrogen, —C(N)—NH—CN, halogen, a single bond, or lower alkyl or alkylene;
E is nitrogen or CR₁₀, where R₁₀ is hydrogen or halogen;
K is nitrogen or CR₇, where R₇ is hydrogen, nitro, halogen, nitrile, carboxamide, carboxyl or an ester;
R is hydrogen or lower alkyl;
R₁ is hydrogen, a lower arylalkyl group having 1-6 carbon atoms, an alkyl group having 1-4 carbon atoms in the aliphatic part and 6 to 10 carbon atoms in the aromatic part, or an aryl group having 6 to 10 carbon atoms;
R₂ is an alkyl group having one to six carbon atoms; a cycloalkyl group having 3 to 7 carbon atoms optionally substituted with halogen; 2,4-difluorophenyl or 2- or 4-fluorophenyl; amino; lower alkylamino; propylamino; N-formyl-lower alkylamino or di-lower-alkylamino; a vinyl group; a 2-fluoroethyl group; a haloalkyl group or a 2-hydroxylkyl group; phenyl or substituted phenyl wherein the phenyl ring is substituted with one or two or three substituents independently selected from C1 to C6 alkyl, halogen, methylenedioxy and hydroxy; alkoxy or trifluoromethyl; 2-, 3-, or 4-pyridine; 2- or 3-thiophene; 2-imidazole; 2-oxazole or 2-thiazole; a pyridyl or adamantyl group; or a benzoxazine group;
R₃ is a hydrogen, amino, substituted amino, halogen or a lower alkyl group;
R₈ is hydrogen or lower alkyl; and X is methylene, O, S or NR₉, where R₉ is hydrogen or lower alkyl.

3. A compound according to claim 2, wherein Z is selected from the group consisting of 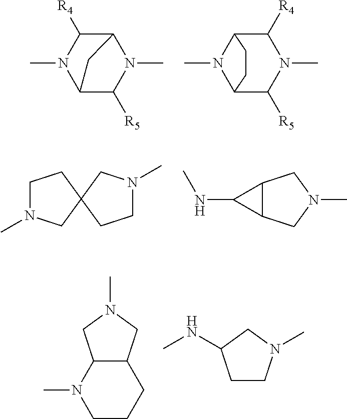
where R₄ and R₅ are independently hydrogen or lower alkyl.

4. A compound according to claim 1, selected from the group consisting of

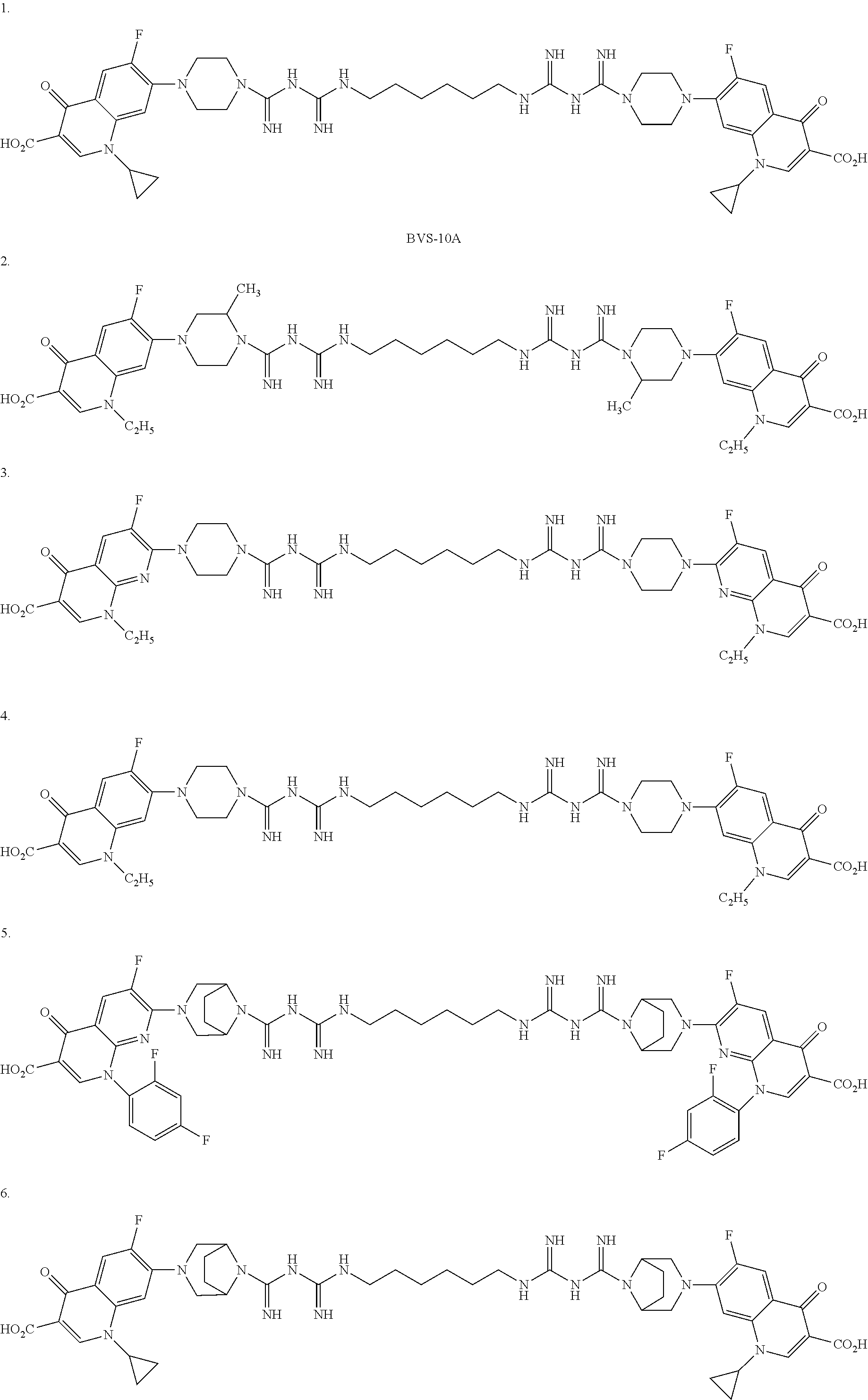

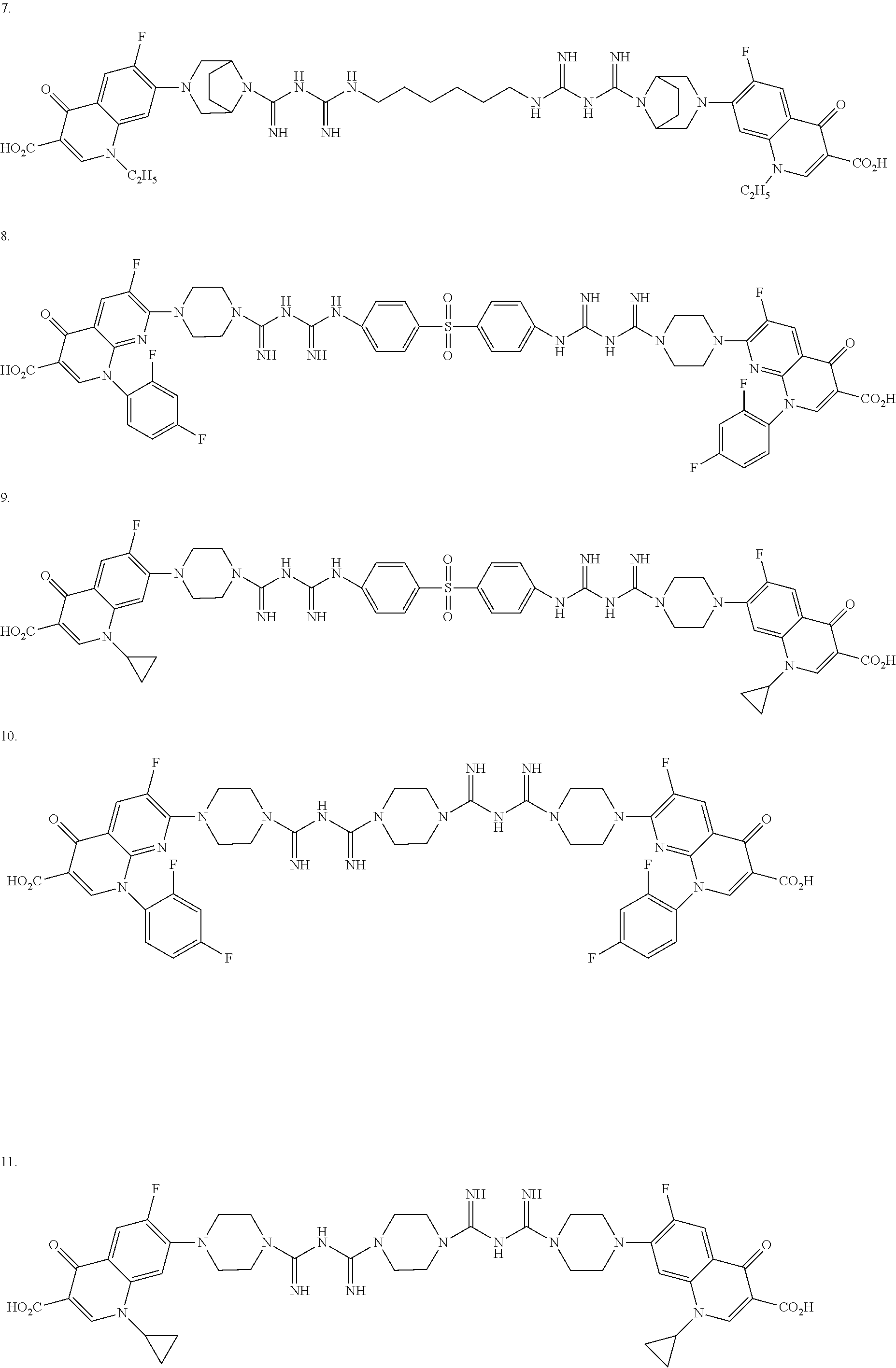

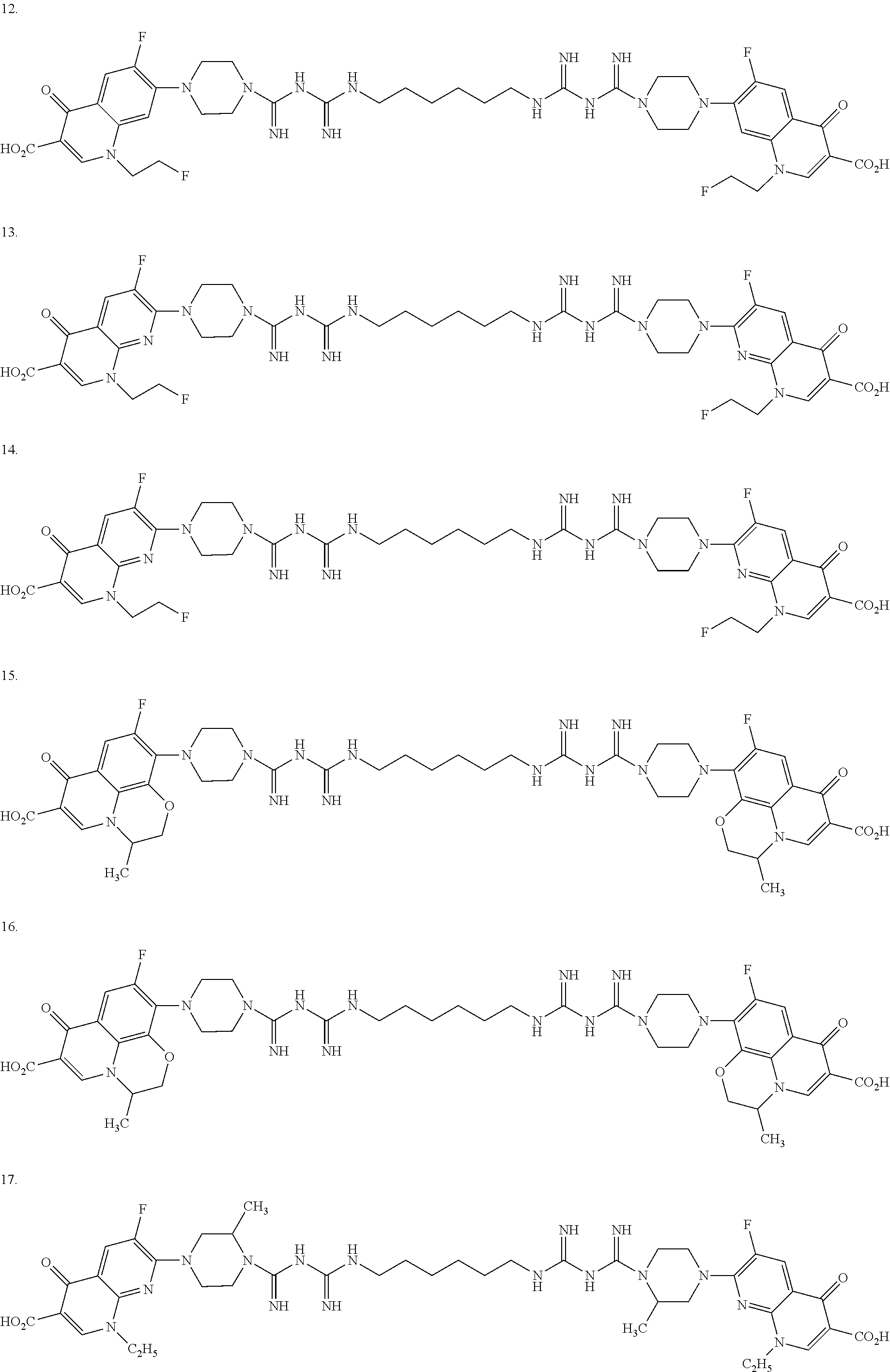

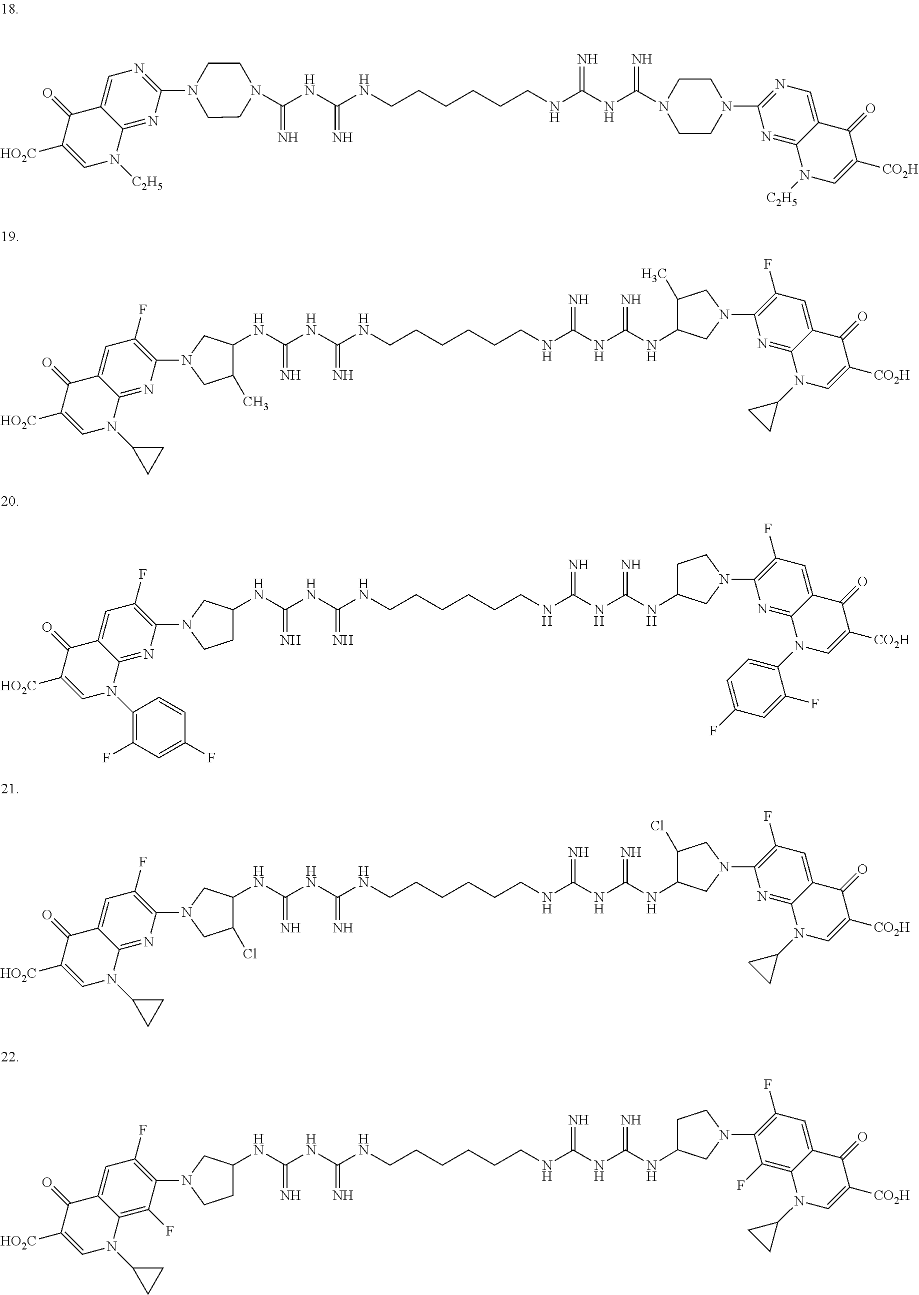

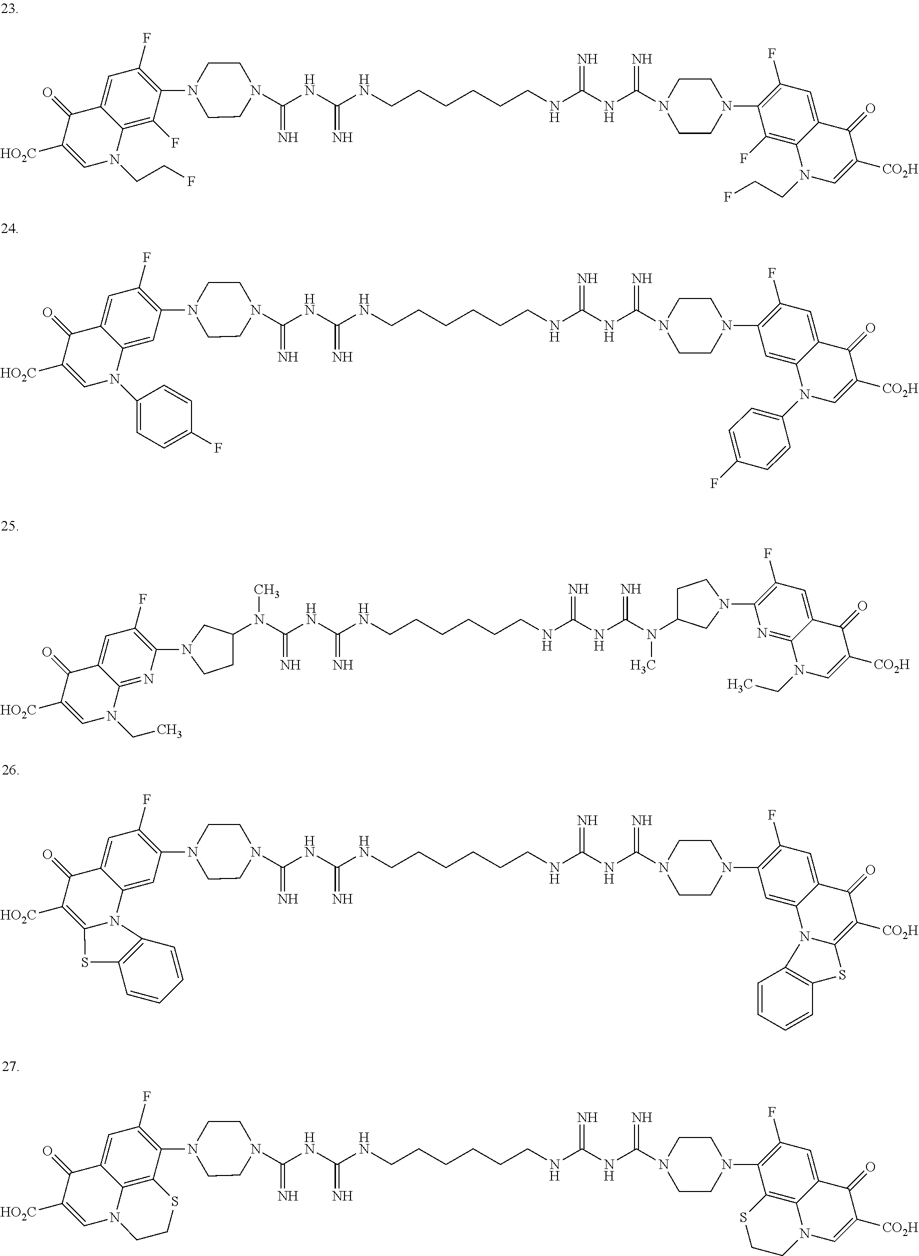

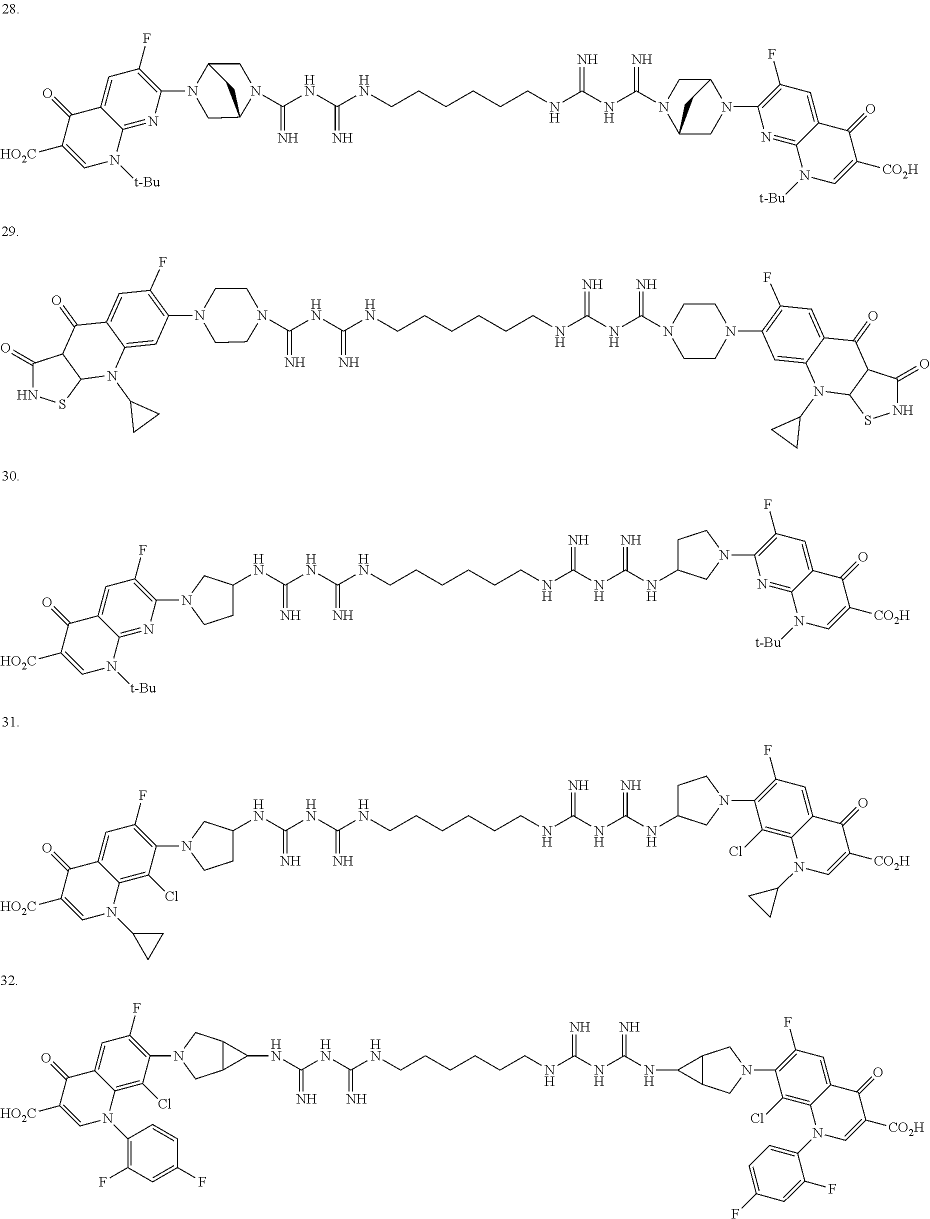

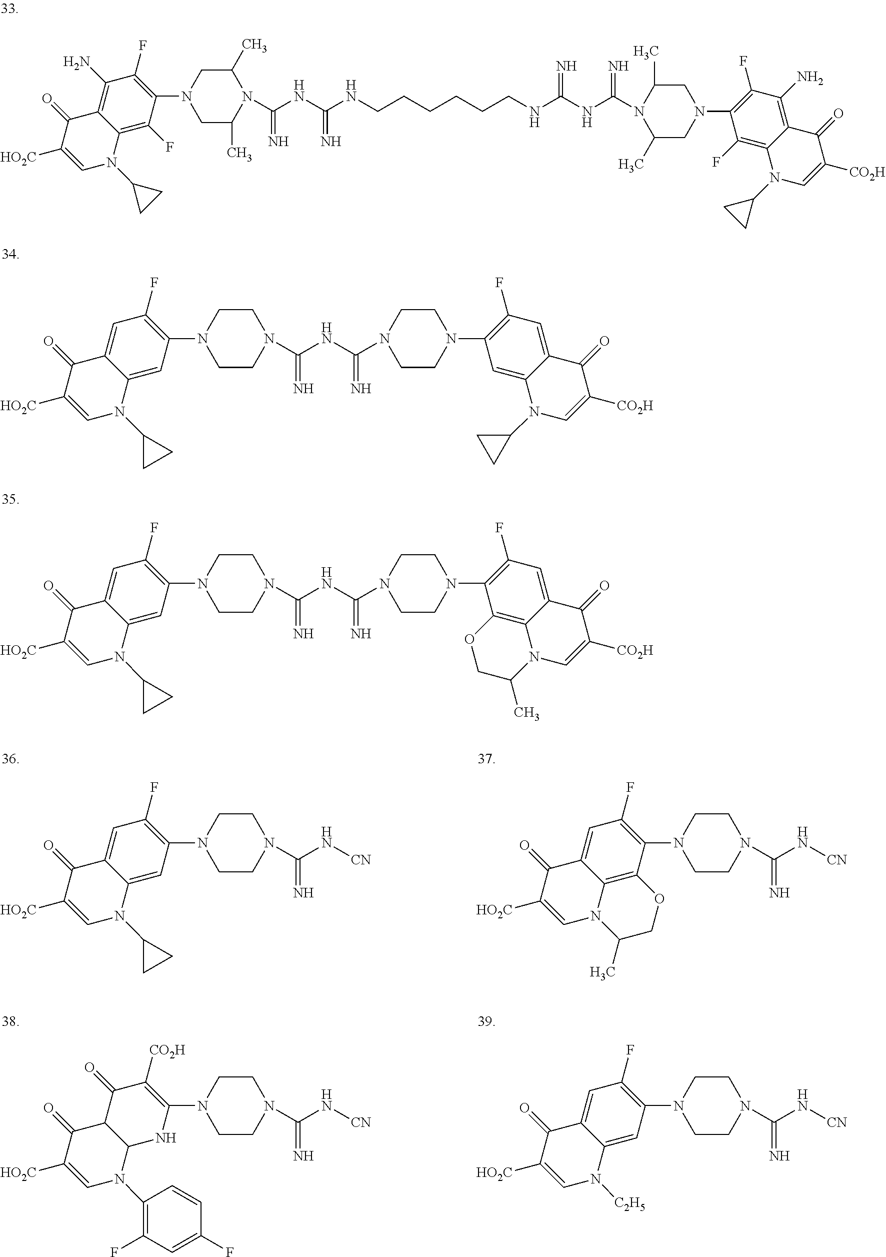

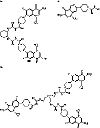 [SMILES: N=C(NC(=N)N1CCN(c2cc3c(cc2F)c(=O)c(C(=O)O)cn3C2CC2)CC1)NC1CCCCC1NC(=N)NC(=N)N1CCN(c2cc3c(cc2F)c(=O)c(C(=O)O)cn3C2CC2)CC1 ; CCn1cc(C(=O)O)c(=O)c2cnc(N3CCN(C(=N)NC#N)CC3)cc21 ; N=C(NCCN(CCNC(=N)NC(=N)N1CCN(c2cc3c(cc2F)c(=O)c(C(=O)O)cn3C2CC2)CC1)CCNC(=N)NC(=N)N1CCN(c2cc3c(cc2F)c(=O)c(C(=O)O)cn3C2CC2)CC1)NC(=N)N1CCN(c2cc3c(cc2F)c(=O)c(C(=O)O)cn3C2CC2)CC1]

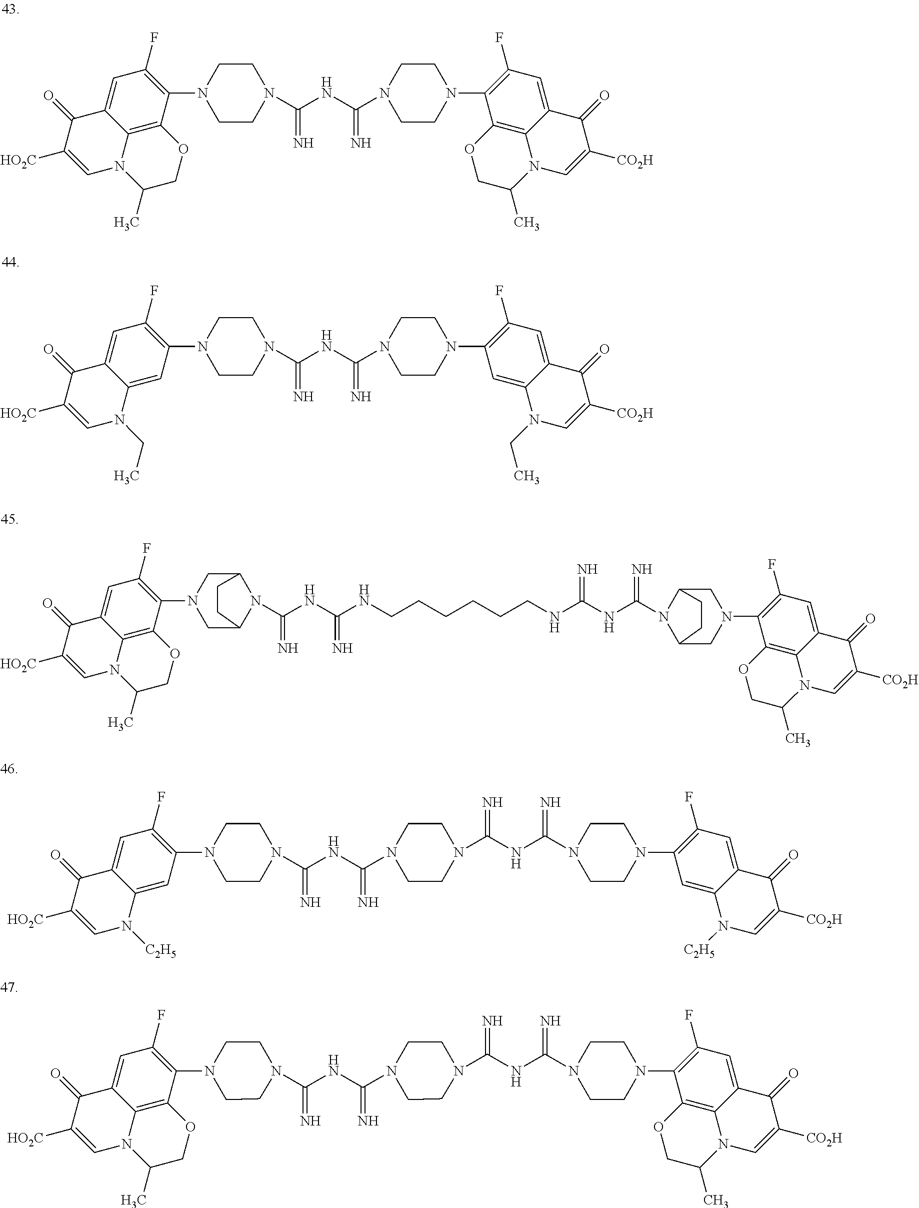

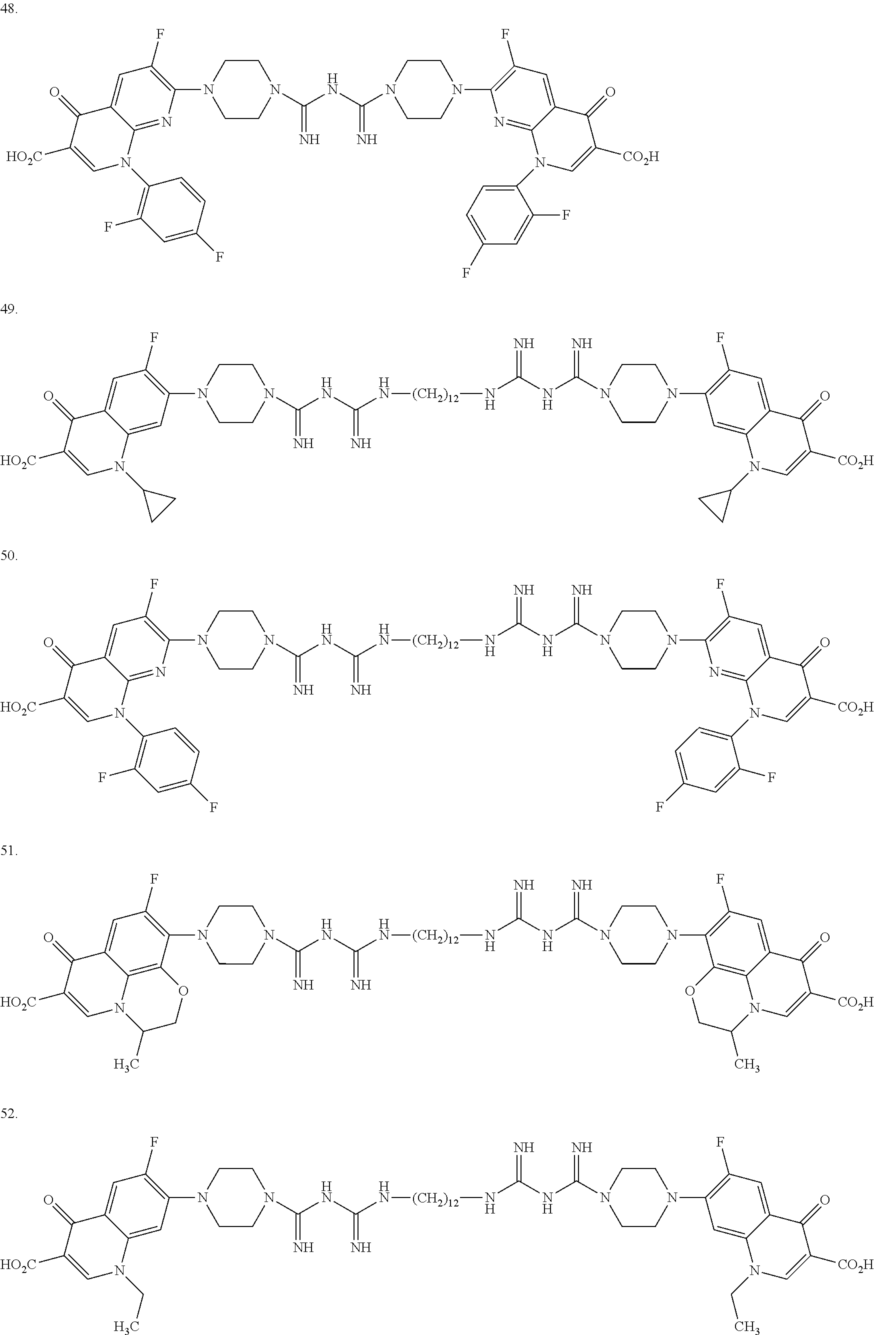

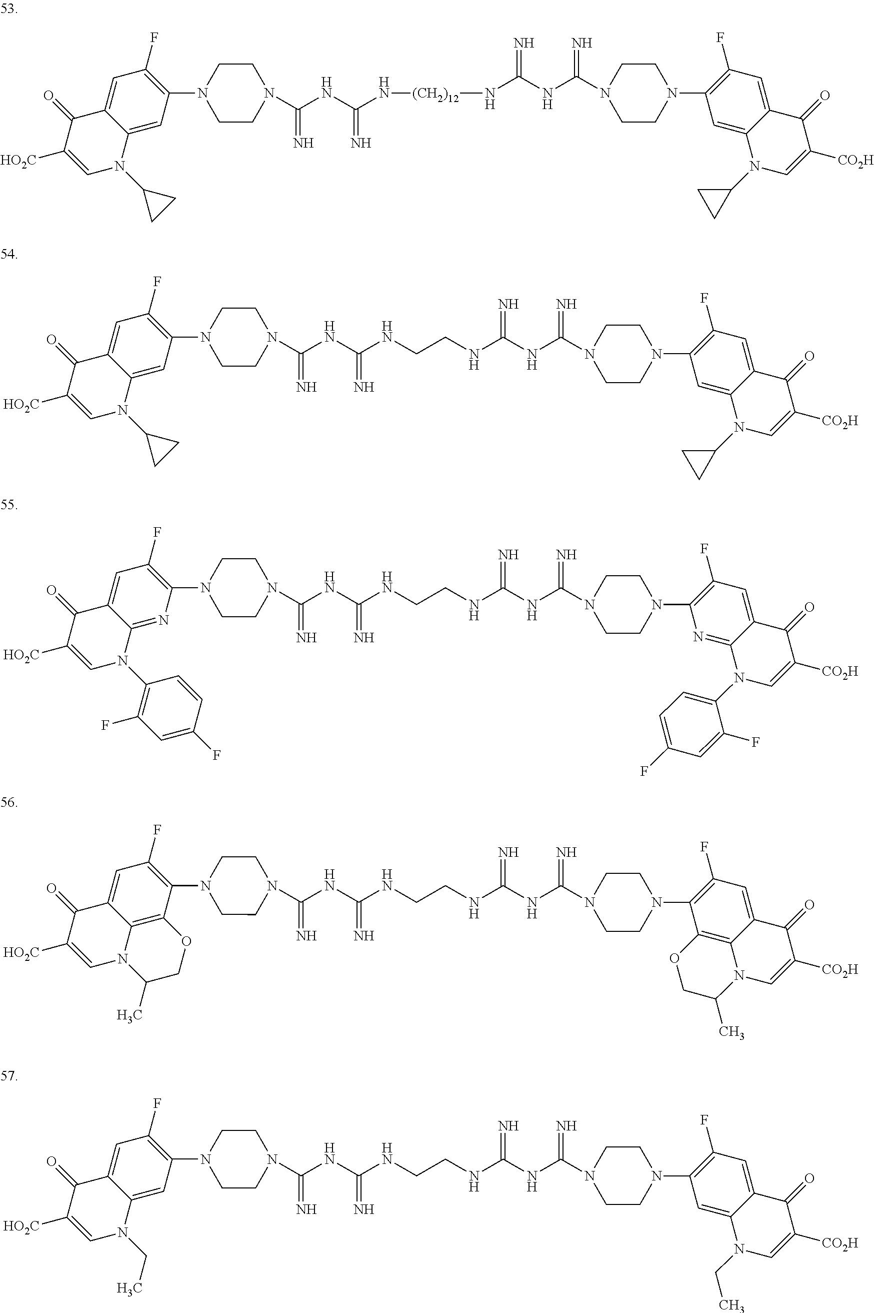

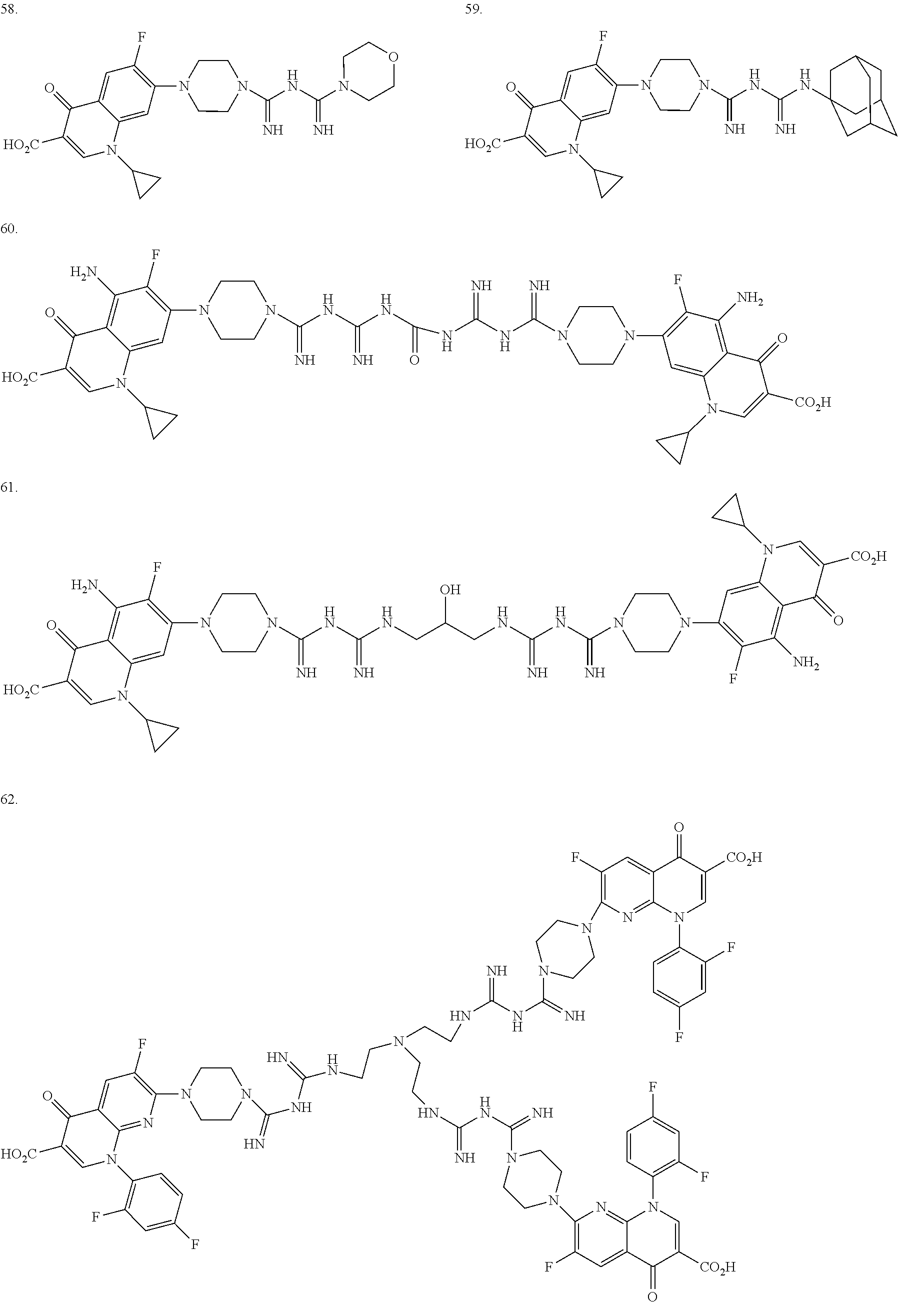

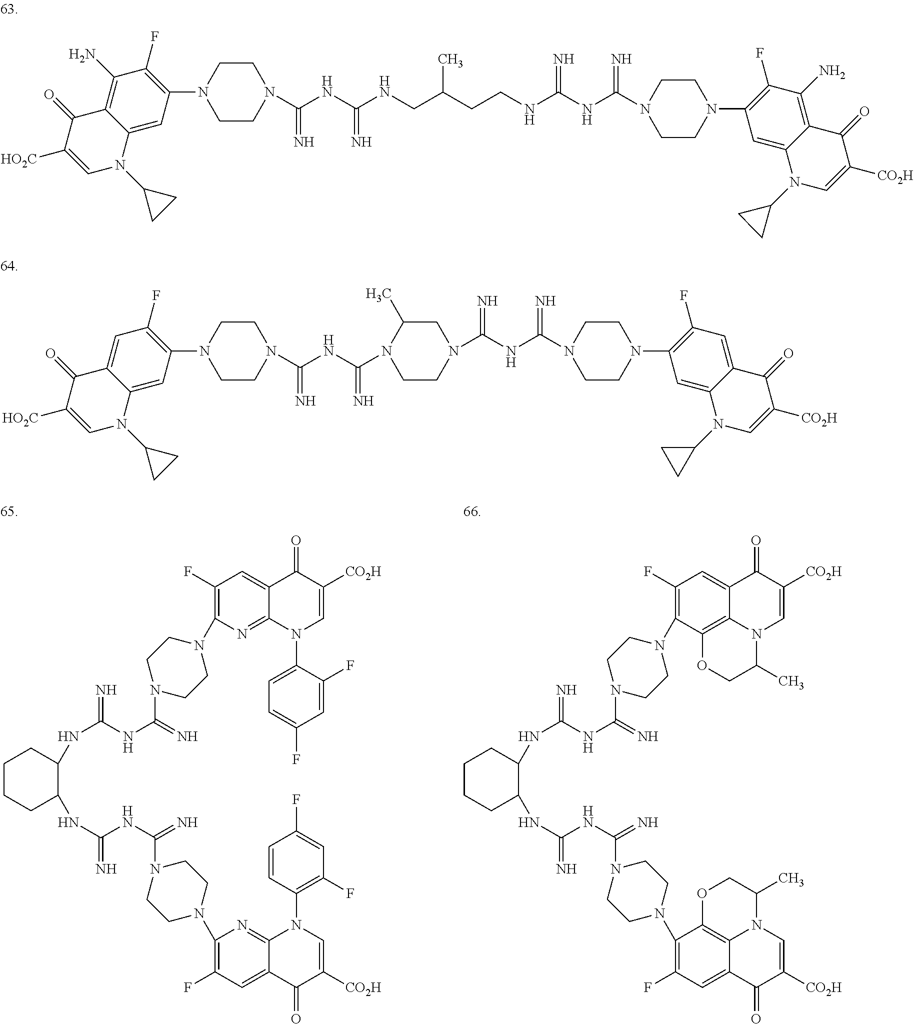

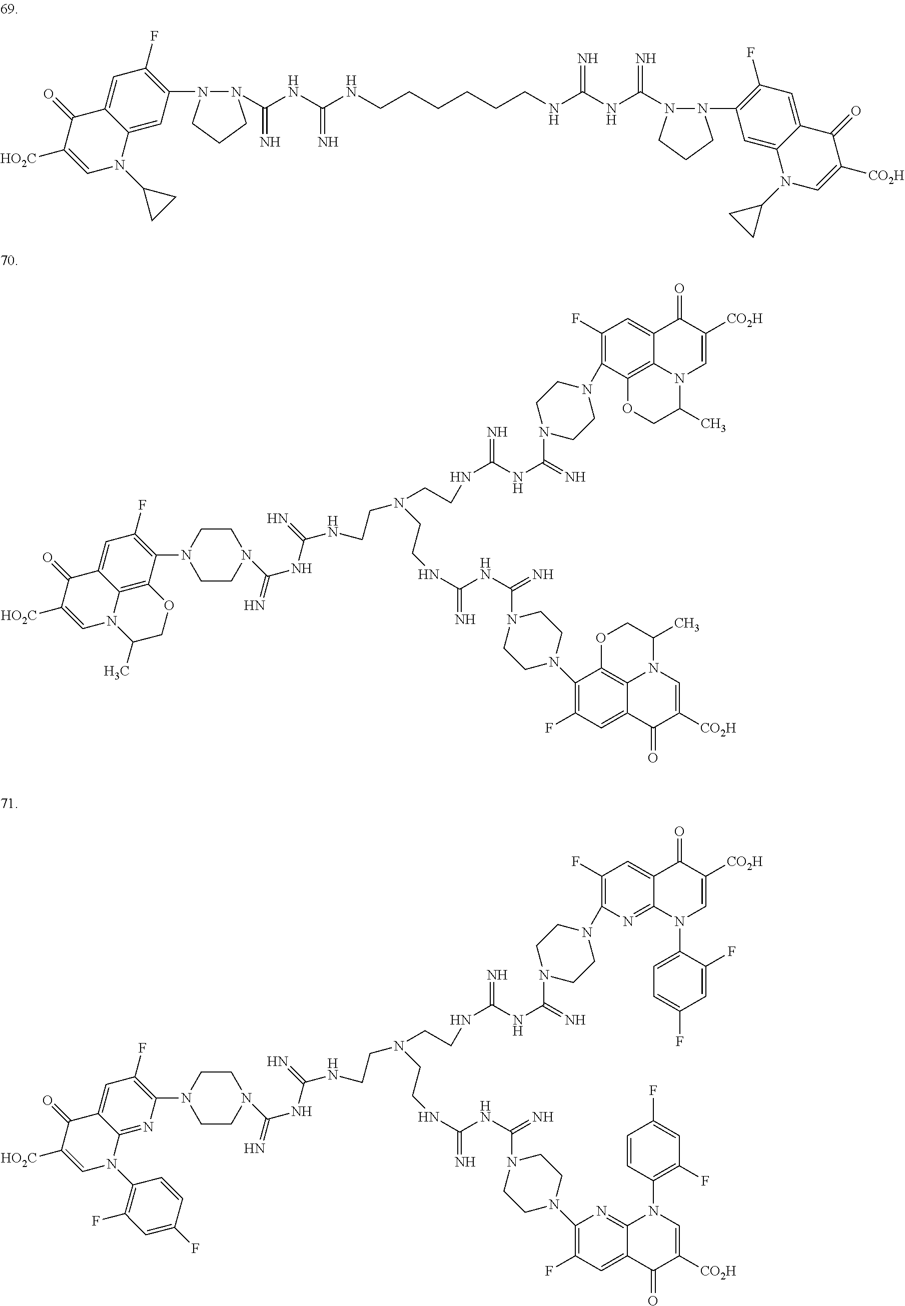

5. A compound according to claim 1, wherein the compound has the following structure: 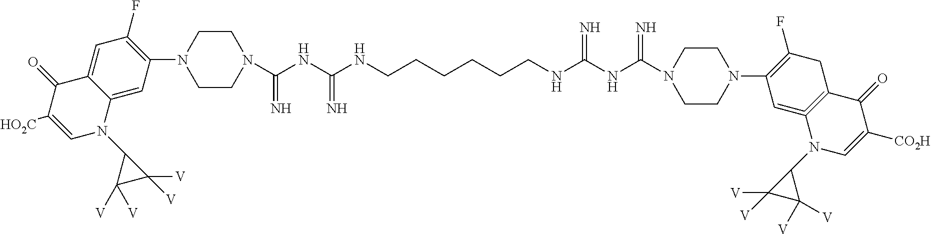
where each V is independently hydrogen or halogen.

6. A compound having the following structure: 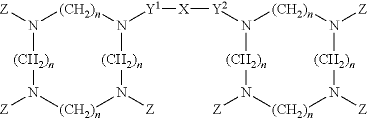
where each n is independently from 1-5;
Y¹ and Y² are the same or different, and are optionally substituted alkyl; optionally substituted aryl; an optionally substituted heterocycle; or a single bond;
X is optionally substituted alkyl; optionally substituted aryl; or an optionally substituted heterocycle; and
each Z is independently —C(NH)—NH—C(NH)-A
where A is the same or different, and is selected from the group consisting of i) hydrogen, ii) a nitrile, iii) an amino, iv) an antibacterial agent, v) an antibiotic, vi) a quinolone, vii) an azaquinolone, and viii) one of the following groups: 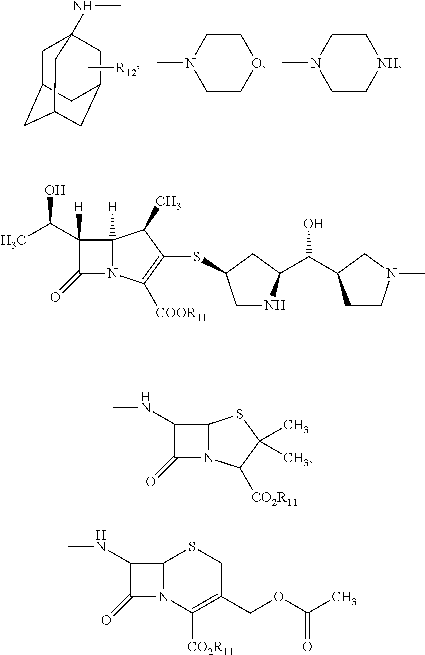
where R₁₂ is hydrogen or C₁-C₆ straight or branched alkyl, and R₁₁ is hydrogen, lower alkyl, an aromatic group or a heterocyclic group
and pharmaceutically acceptable salts thereof.

7. The compound of claim 6, wherein both Y¹ and Y² are single bonds.

8. The compound of claim 7, wherein X is C₁-C₁₀ alkyl.

9. The compound of claim 6, wherein Y¹ and Y² are lower alkyl and X is phenyl optionally substituted with halogen and/or lower alkyl.

10. The compound of claim 9 wherein Y¹ and Y² are —CH₂—

11. The compound of claim 6, wherein Y¹ and Y² are lower alkyl and X is pyridyl optionally substituted with halogen and/or lower alkyl.

12. The compound of claim 11, wherein Y¹ and Y² are —CH₂—.

13. A compound having the following structure: 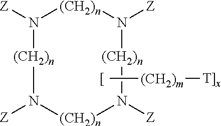
where each n is independently from 1-5;
each m is independently from 0-12;
each Z is independently —C(NH)NH—C(NH)-A,
where each A is the same or different, and is selected from the group consisting of i) hydrogen, ii) a nitrile, iii) an amino, iv) an antibacterial agent, v) an antibiotic, vi) a quinolone, vii) an azaquinolone, and viii) one of the following groups: 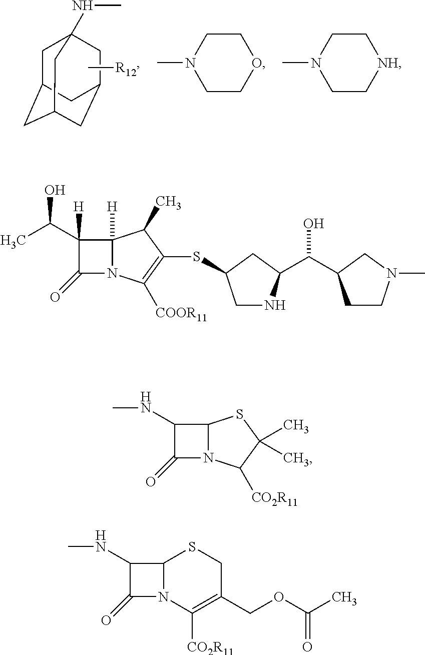
where R₁₂ is hydrogen or C₁-C₆ straight or branched alkyl, and R₁₁ is hydrogen, lower alkyl, an aromatic group or a heterocyclic group; and
T is hydrogen, lower alkyl optionally substituted aryl or an optionally substituted heterocycle; and X is from 0-8.

14. The compound of claim 13, wherein the compound has one of the following structures:

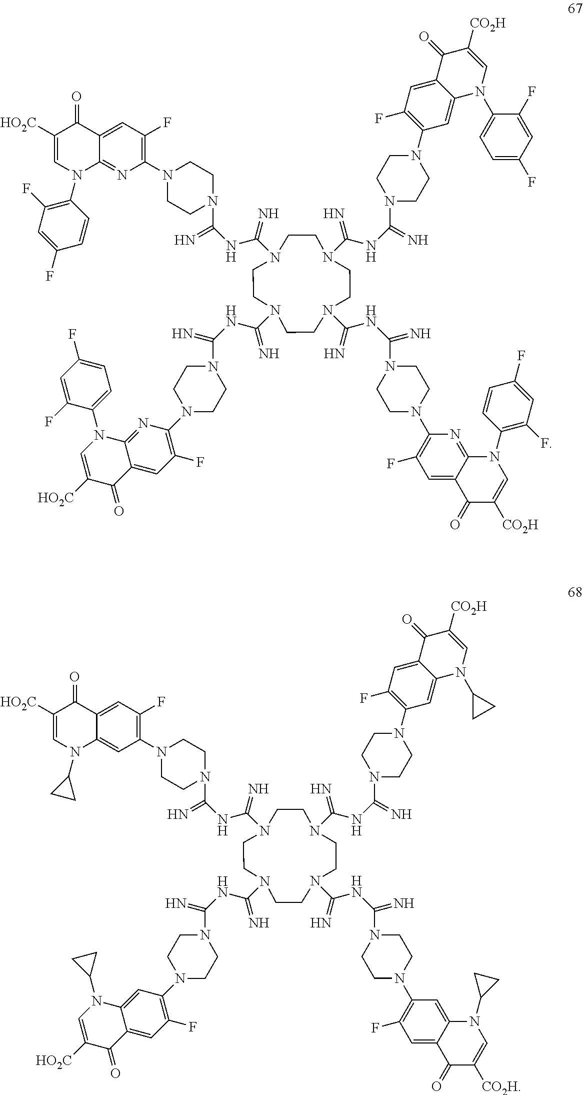

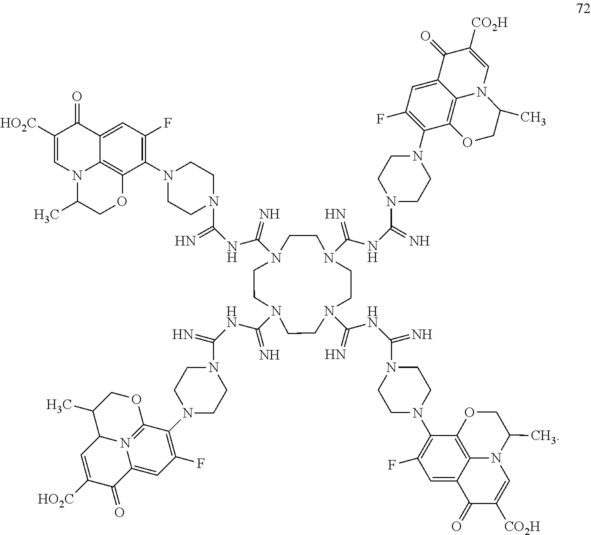

15. A compound having the following structure: 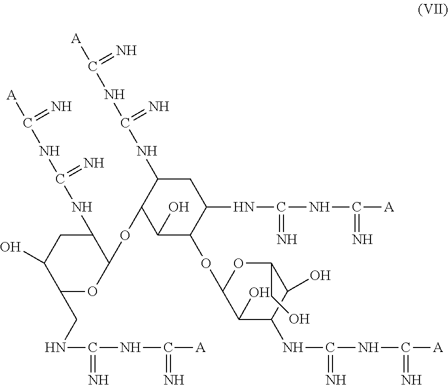
where each A is the same or different, and is selected from the group consisting of i) hydrogen, ii) nitrile, iii) an amino, iv) an antibacterial agent, v) an antibiotic, vi) a quinolone, vii) an azaquinolone, and viii) one of the following groups: 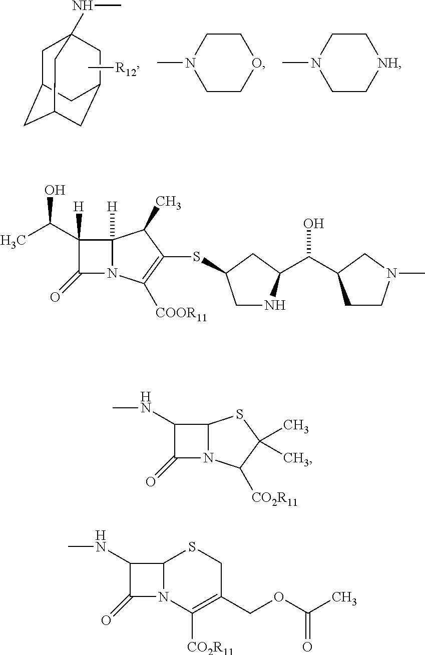
where R₁₂ is hydrogen or C₁-C₆ straight or branched alkyl, and R₁₁ is hydrogen, lower alkyl, an aromatic group or a heterocyclic group; with the proviso that at least one A is not hydrogen.

16. A compound having the following structure: 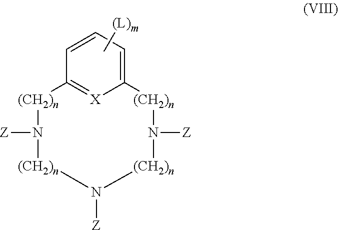
wherein each n is independently from 1-5;
m is from 0-3;
each L is independently hydrogen, lower alkyl, optionally substituted aryl, or nitro;
X is CH or N;
each Z is independently —C(NH)NH—C(NH)-A where
each A is the same or different, and is selected from the group consisting of i) hydrogen, ii) a nitrile, iii) an amino, iv) an antibacterial agent, v) an antibiotic, vi) a quinolone, vii) an azaquinolone, and viii) one of the following groups: 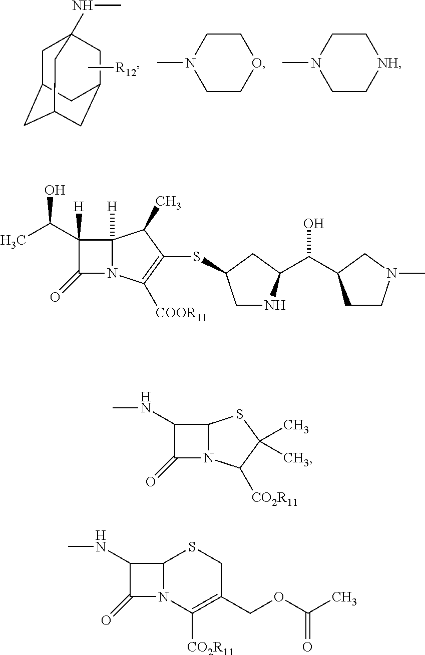
where R₁₂ is hydrogen or C₁-C₆ straight or branched alkyl, and R₁₁ is hydrogen, lower alkyl, an aromatic group or a heterocyclic group.

17. A compound having the following structure: 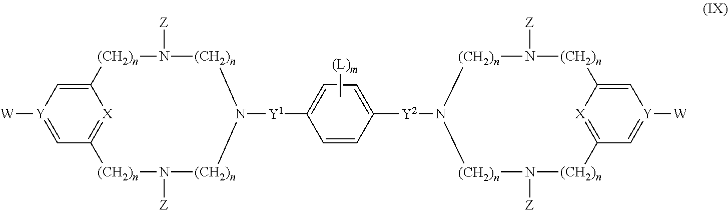
where m is from 0-4;
each L is independently hydrogen, halogen, alkyl, aryl or nitro;
each W is independently hydrogen, halogen, alkyl, alkoxy, or aryl;
X and Y are each independently CH or N;
Y¹ and Y² are each independently optionally substituted alkyl or a single bond; and
each Z is independently —C(NH)—NH—C(NH)-A,
where each A is the same or different, and is selected from the group consisting of i) hydrogen, ii a nitrile, iii) an amino, iv) an antibacterial agent, v) an antibiotic, vi) a quinolone, vii) an azaquinolone, and viii) one of the following groups: 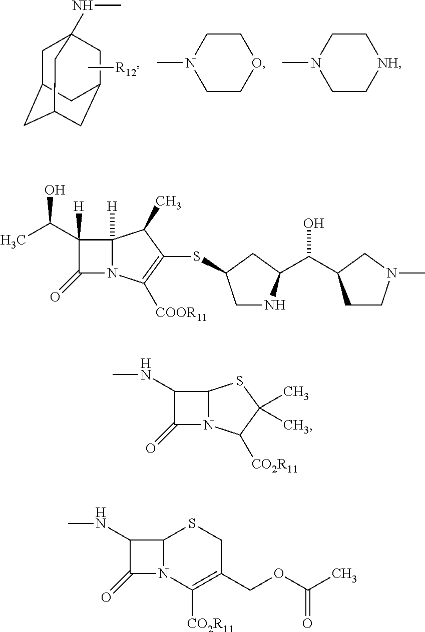
where R₁₂ is hydrogen or C₁-C₆ straight or branched alkyl, and R₁₁ is hydrogen, lowered alkyl, an aromatic group or a heterocyclic group;
and pharmaceutically acceptable salts thereof.

18. An antiviral and/or antibacterial composition which comprises:
a) an effective amount of a compound according to claim 1;
b) a pharmaceutically acceptable carrier.

19. A method for preventing or treating a viral and/or bacterial infection in a mammalian host, said method comprising administering to a mammal in need thereof an effective amount of the compound of claim 1.

20. The method of claim 19, wherein said mammal is infected with Yersinia pestis, Bacillus anthraces, Francisella tularensis, Burkholderia pseudomallei, and Burkholderia mallei, Escherichia coli, Pseudomonas aeruginosa, Plasmodium falciparum, Mycobacterium tuberculosis, influenza A H3N2, influenza A H1N1, HIV, or a combination thereof.

21. The method of claim 19, wherein said mammal is suffering from a bacterial or viral ocular disease.

22. A method of treating an ocular disease in a patient in need thereof comprising administering to said patient an effective amount of the compound of claim 1.

23. A compound having the structure: 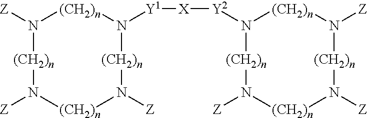
where each n is independently from 1-5;
Y¹ and Y² are the same or different, and are optionally substituted alkyl; optionally substituted aryl; an optionally substituted heterocycle; or a single bond;
X is optionally substituted alkyl; optionally substituted aryl; or an optionally substituted heterocycle; and
each Z is independently —C(NH)—NH—CN or —(CH₂)ₘ—NH—C(NH)—NHCN where m is from 1 to 6.

24. A compound having the structure: 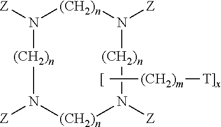
where each n is independently from 1-5;
each m is independently from 0-12;
each 2 is independently —C(NH)—NH—CN or —(CH₂)_{q}—NH—C(NH)—NH—CN where q is from 1-6, and
T is hydrogen, lower alkyl optionally substituted aryl or an optionally substituted heterocycle; and x is from 0-8.

25. A compound having the structure:

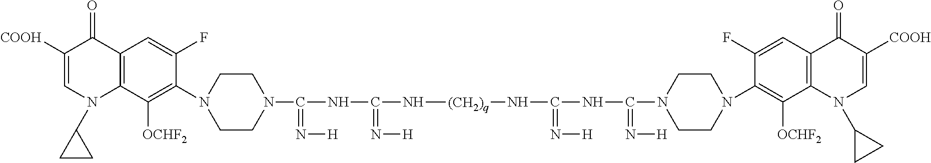

26. A compound selected from: 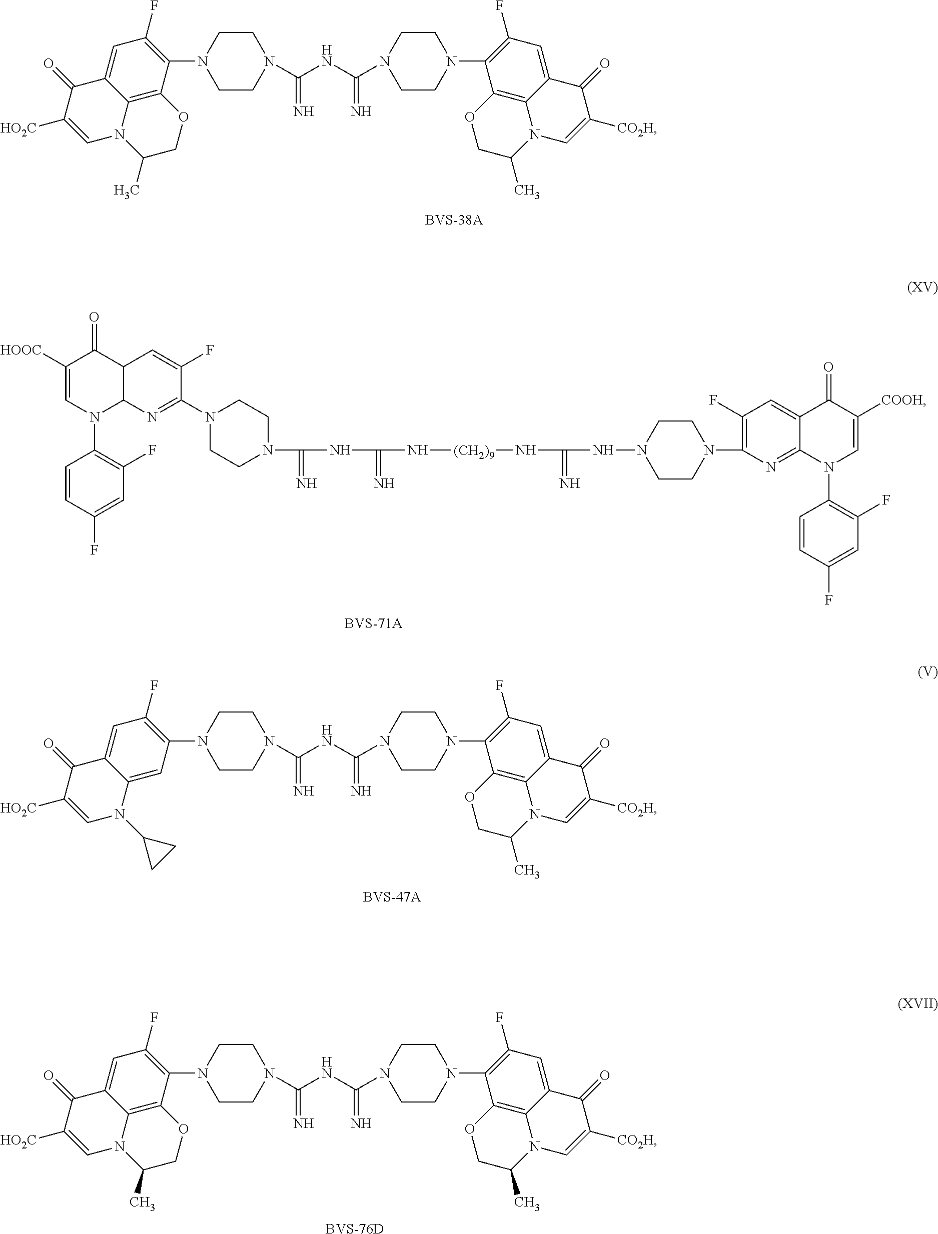 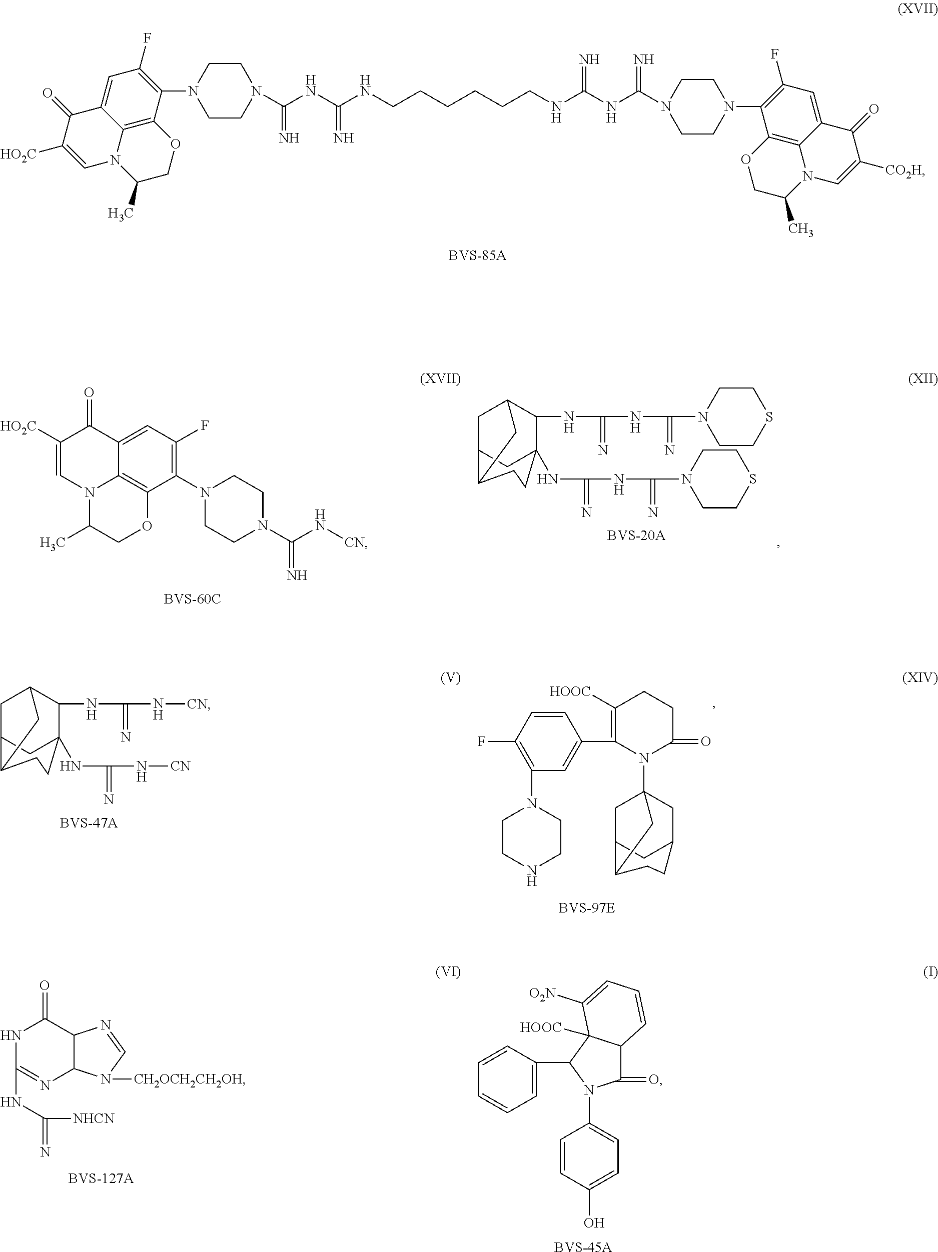
or a salt, prodrug or derivative thereof.

27. An antiviral and/or antibacterial composition which comprises:
a) an effective amount of the compound of claim 26;
b) a pharmaceutically acceptable carrier.

28. A method for preventing or treating a viral and/or bacterial infection in a mammalian host, said method comprising administering to a mammal in need thereof an effective amount of a compound selected from: 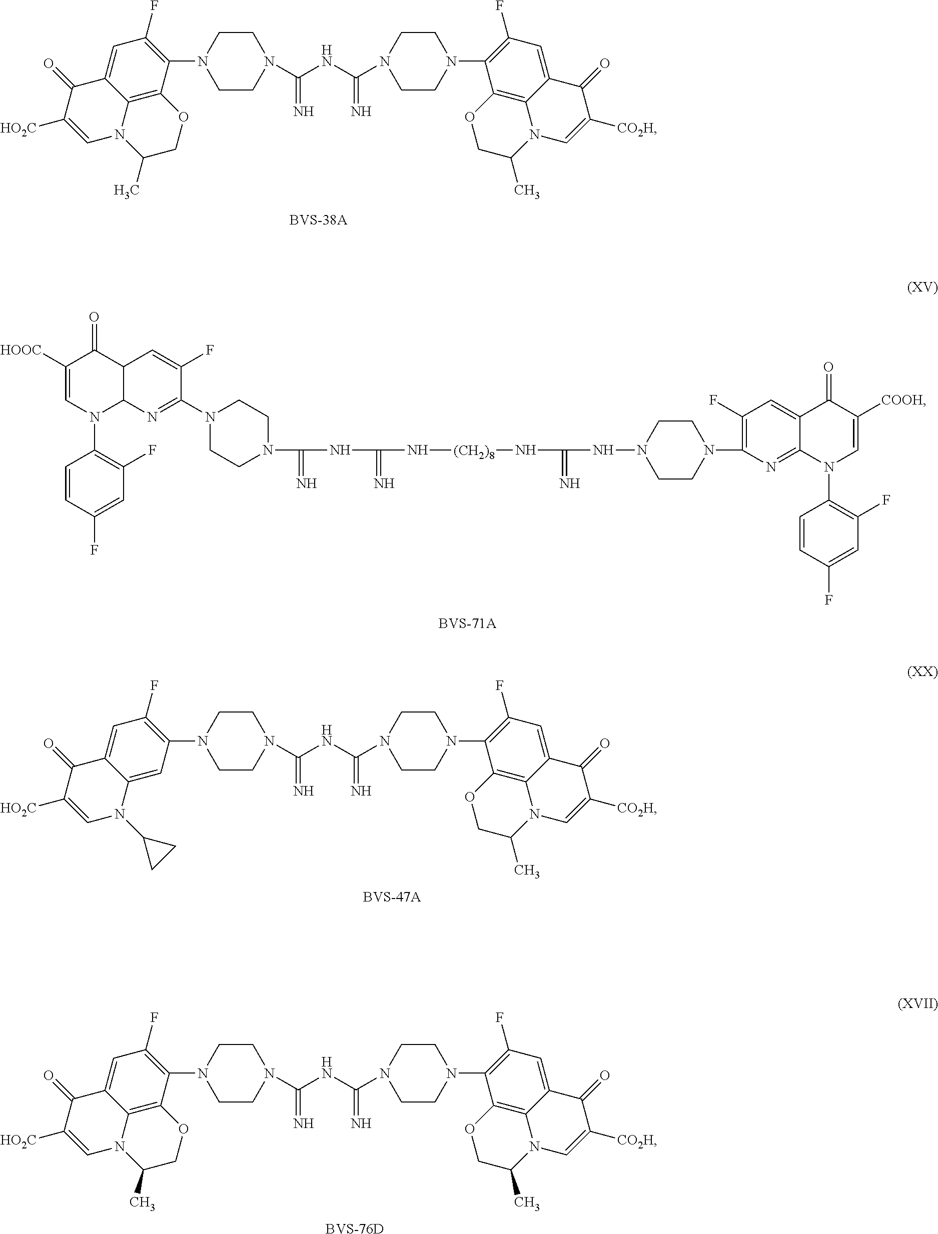 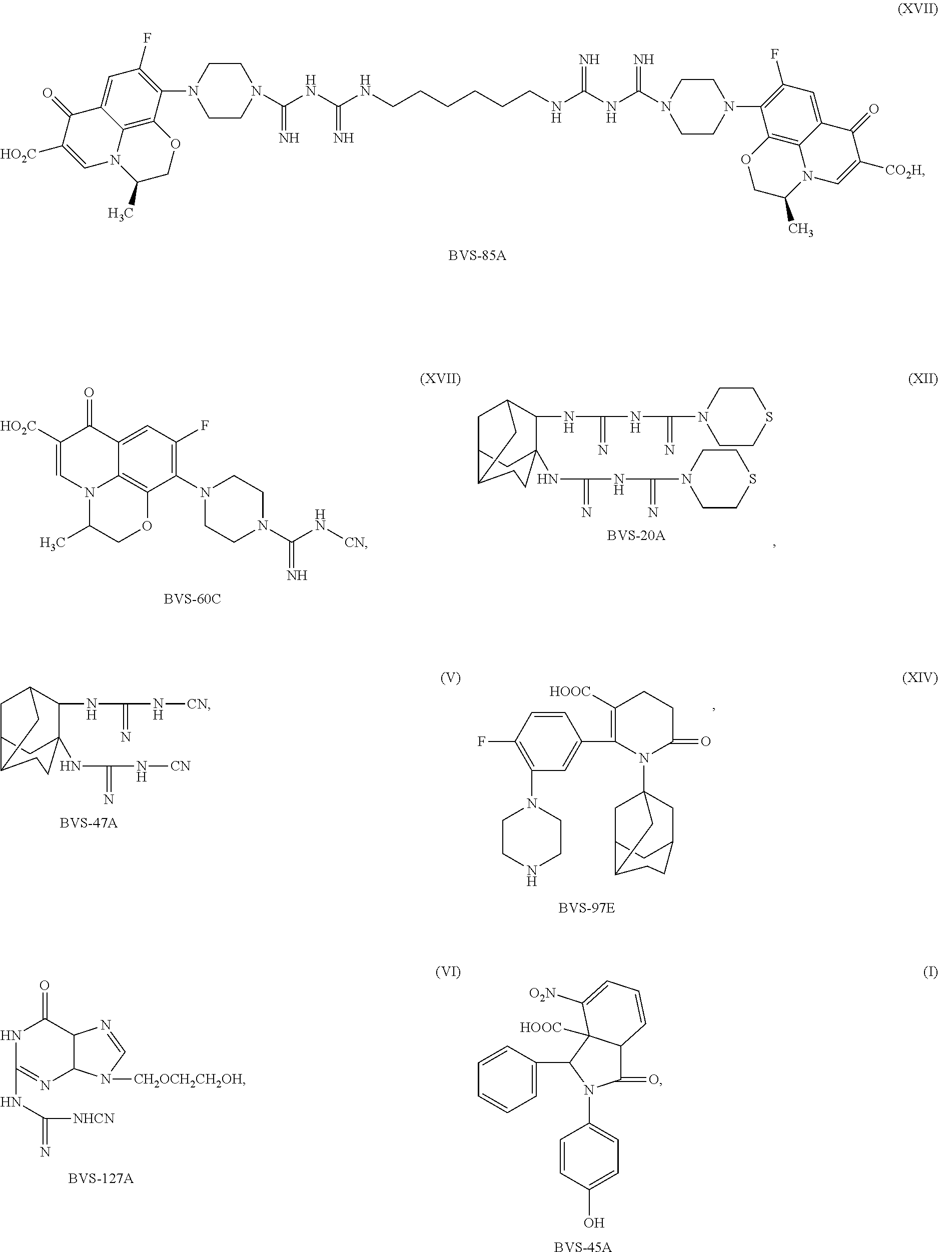
or a salt, prodrug or derivative thereof.

29. The method of claim 28, wherein said mammal is infected with Yersinia pestis, Bacillus anthracis, Francisella tularensis, Burkholderia pseudomallei, and Burkholderia mallei, Escherichia coli, Pseudomonas aeruginosa, Plasmodium falciparum, Mycobacterium tuberculosis, influenza A H3N2, influenza A H1N1, HIV, or a combination thereof.

30. A method of treating an ocular disease in a patient in need thereof comprising administering to said patient an effective amount of a compound selected from: 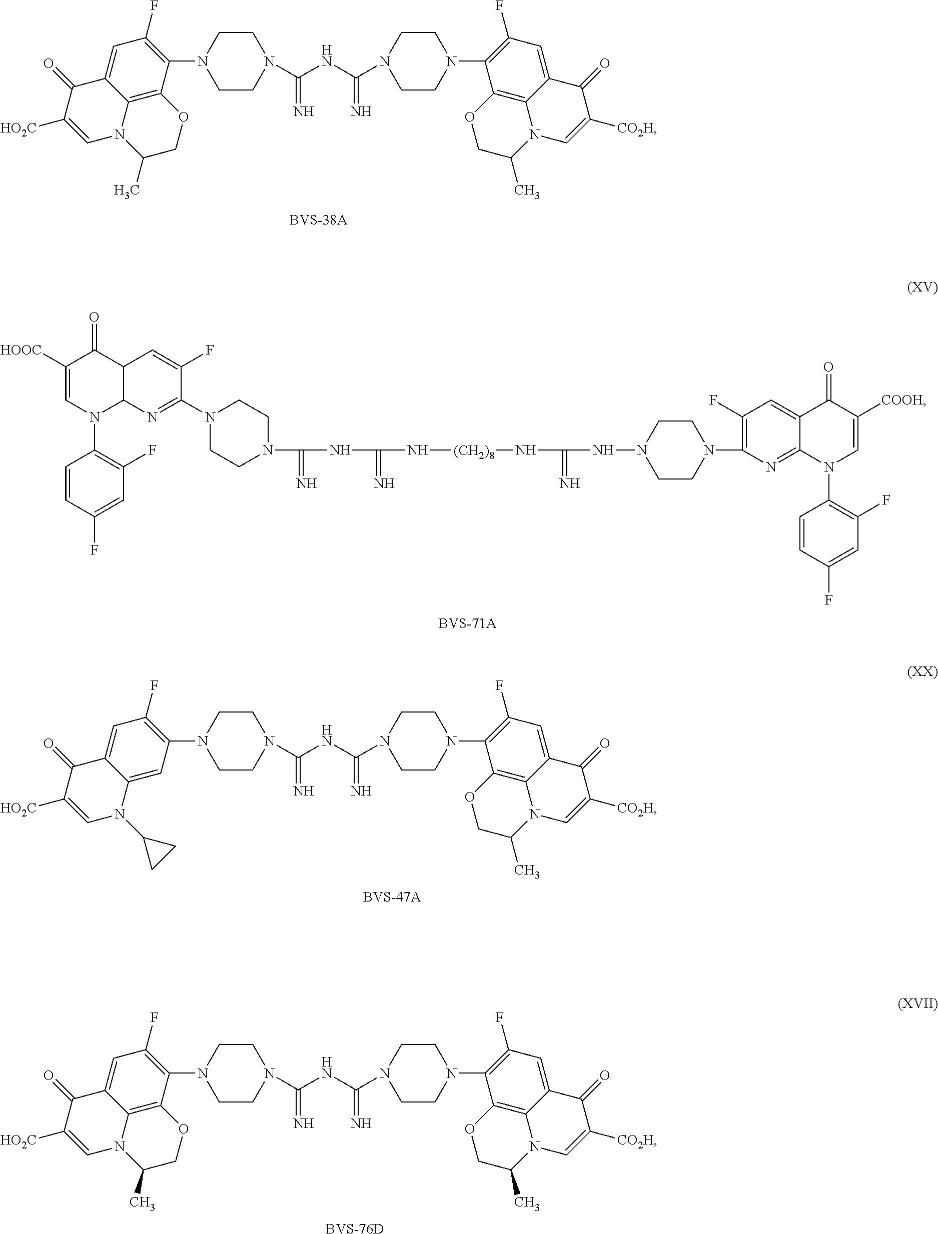 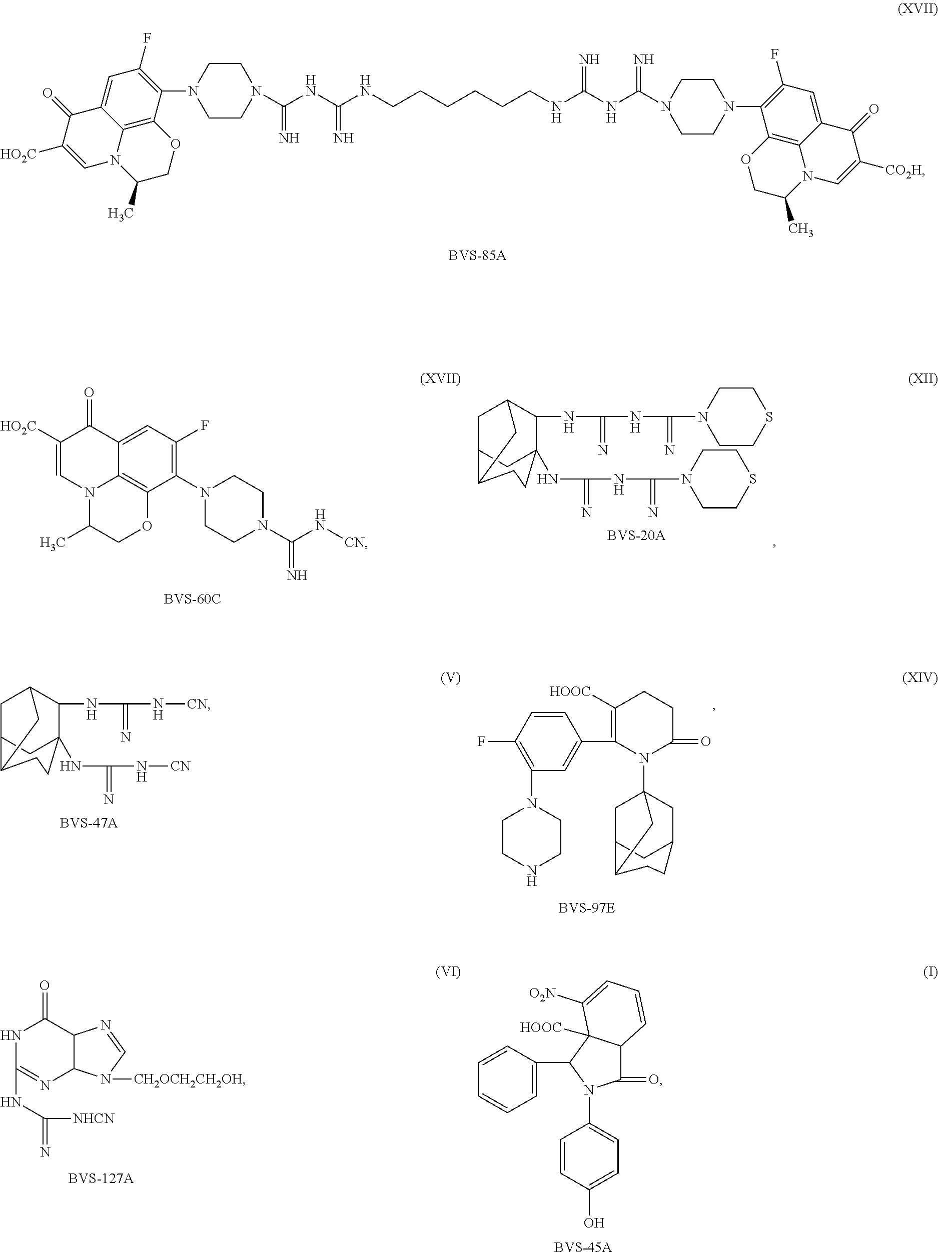
or a salt, prodrug or derivative thereof.

31. The method of claim 30, wherein said ocular disease is bacterial or viral conjunctivitis.

32. A salt of BVS-10A 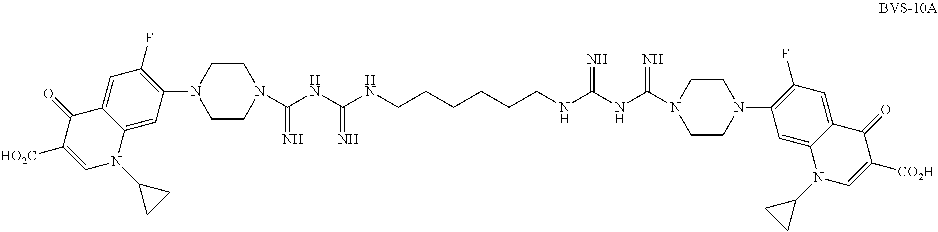 [SMILES: N=C(NCCCCCCNC(=N)NC(=N)N1CCN(c2cc3c(cc2F)c(=O)c(C(=O)O)cn3C2CC2)CC1)NC(=N)N1CCN(c2cc3c(cc2F)c(=O)c(C(=O)O)cn3C2CC2)CC1]
in combination with phosphanilic acid.

33. A prodrug having the formula: [chemical structure]
